SEQUENCE LISTING (69 sequences)

SEQ ID NO: 1 - DNA, 5917 bp, Oryza sativa
ctagtagtctgcatcccacacagagaatgcactagaggacttcatccacttggcagtgatttcagggtagtgccggttgatcacatctcgtaggccttctgttgttttgacccattgcatccccttcttggtgtatgtttcctcattgaaattgcttgtaaaaaatcgatctgcttcgagcctcctgtttttagagatgttaaattaatattcatatcttaactgagtggttatcatctataattagtacttccacatttgacttctttttttttctggtaacattctggatctaccagtaatgtacccatatataatggtcacatcgttatattttcttgccttaataaactttggtttgggtaaattttgaatcactcaaaataagcttaagccacattctcggttgttgcctaatacgtttagcaagggcttaaacaaaaattggtactttgtctagcattcaatattagttagaactaaatgttaccttgatgccattaaaatgaaaatgttgaatgctgtttcgcttatggcaaatcccttgatttttttctctgccataagaccaacaagaagatccaatttctctacatcatctccgtatattgctcttatggtttcaattgcatccttgtcacttgtcagatcctcccagctttttattggaatcagaaatagtctcctcctgaactcattgtaccttggtacacttctctccctgtctcggtatactttaatttagaggaaaagaaaatgatattaatctcagtgtaatcattgtcaaaaggcattagacacagcacaatgtcctctttacttaacttggatagtacagtttttgcttaacatttttgtttatactagtacgcagcaatgtccttgttttttagataaatgctagttaaggttacaacatacaaacatgatatatgtctcggtaagagaatcttgtattttcaatctggttagtacaataatagttcgagaattcttgaaaattgaaaagtatgttgtgataaatttcaacatagtagtttaattaaggtcacaacacttgttctagcacttcatttgttgccagtgtgagttattttcaatatttttatgcattggattattcttggtggaccctagcttgtaccaacttgacatggtgcaaaatcatgatcctctactggtagtggtataaattgagtctaaattttaattataccttctagggcagcaaggtcgattctgtcagatcgattggtaccatccaaattttgtggaatgaggttcctgaagaaacttgggtagttccatagttcaagtgcaccacaagcttgatatcccattgataatgcttgtttttcaaaccctatttttgatagctgttcttcccccttgagaccagtcatctctccaatatcgatgctacattttgacacaagaaagaaatggtgtcggtgtttatgtcagttatactaaaaatgtgctaactgagcagaaaacttattgtgaccaagattatacattctactttttattgtgtagctaagaagctaaaaaaataataaaaaatctatctgtttgtgtcgagttcagtgtctaaagtcaggttcagtaatccatgatgtaattaagagtcgatatgtaatagaagaacgtcttaactttttttttcgttatttatttataatctactatttttctgtctgatttaataaatagaagttgttagtttgcatttcatcttccacattaaaaaaaatgagagggatggatgccaaacataaggccctgtttagttcccaaaaccaaaattttcacgctgtcacgtcgaatttttggacacatgcatgaagtattaaatatagaaaaaacaaattacacagattgcgtgtaaattgcgagacgaatcttttaagcctaattgcgctatgatttgataatatggtgctacagtaaacatttgctaatgatggattaattataggcttaataaattcgtctcgcaatttcctagcgaaatatgtaatttgttttgttattagactacgtttaatactttaaatatatgtccgtatatccgatgtgacaaccaaacctaaaaatttttcccaactaaacgaagcctaaattgtttactcactcttccaggtatggtggggaattatttgcatcgggttgccctgtcggatccctgagcttgagagtgcttggtattagggagtgcattctgtaaacactggtaaactcttcagtcaaagaataaggtacgccatggttgttcggcttcttcaatcccacaagcccacctaatatcggtcctcctatgtgaccaaatgtgtcttttattttcttacccaataatccataccttcacaaaagaatcagaatatcaggtaaattgtacacattctcactaatttaattaatttgaatttgcagatgtaaataaaatttccacttggaaaagtaatacccgcatcaatggttatattttttttctttcggtaaatatgagaaaatgagataaatgtatcctcaccaatttgcgcgcattgcagctctcatggtttttgtcttgagaagctcgacagtccaatcaatagtgtgaacctttgcaataacagcagaagtgaccaatttggcataccgatacaattcttcgtccgataggttgggatgttcttcctgagattaaaacaacatttgtaaaaaaaaaatttcatatttttagcattaagaaaacaaattaatgttttggtaaataacagtttatattcttcctaaaactaaacaaatagacaatcagtggcaagctagtcaagaacagcaaattatattcttcctaaaactaaacaaatagaaaatagtgatgatatattcgcttacctttattgcatcagaaactgcattgtgttccttaacaaaaagagcttgtaagatcgaaacaccagcccaactgttgcgaatgtcgccggataatgccacaccgttctccttatgcaaaagaagaccgtcatctccgatcactagttttccatcaacataagtcctgagcttttctgccctttcctcattattaccatatactgcactcccatcccttggtatatataattatagaaattgcaaaaaaaaaaattacgacattttttaagggcaaatatgcattgatttatatatatacatcgattgtctctattttttaagttcattctttgtgcttaagataaatttttgataaagatcttaactcgagaaaaccttaagcattactcaccaccaagctgtgcggacattgtagtgaccaatctttattccatcagaattcgtaggcagttcttttgtgggatggaacttgaacgatttgagtgggcactcattggccacttcttttggagctgtgatttcaatctagaagaaaccaagaaaaaattgaattatatatcaactagcaattagaggtggtagaacccaacccgacccaagaaacctgctcgactcgactcgaatctgacctgaccagacatgacgttttccaaaaaatcaaattgacctgacccgacctgatatgatccaaaactgacccgatccgagatcaaatagcttccaatccaactcaaatcaacatactataaaaaaaagttgatattgaaatcggctcgatccgacccgacccgagatattctagaaggtcaaagtaacccaacccgtccaacccgaacctgacccgcccgcccgatttgccacatgtaactacaatatatatttcaaattctcaatggaagatgaagacttatttttataacttcaacaacttaaggctctcaaattaaggaattaaatcaagcattcacctgtccggtatcctccatatgatccatccagtcatgaaccatgaactgtatccatgcagctgctagtatattgaactgtttccctgtgtccttgtattctctcctggctaacagctttgtcgccacaacaaatggatctgggctcatcaactgcgtcgataaataacagctaattattttatgcataattaacctgatgatttaaaagtgtacctcaaatttccatctgttaacagtatgatctaattaaaatgaaaatggttctatttttaaattaacaagcacattagctcttataataatatatactgacaaaactcccgtcttatttgaagcatgcagaacgaactaccaaacgaaagtatcataaaaaagagtgtcattataaagtcttaaagtcaaactgtcaaactagatattaaataagaaataaaaatcaaacactttttttggtgatctttactccattatttttcattaactaaattcaatatatatggtatctaattttctattgaacaagataatagggatgaaaattgaaatcagctggtaaatatagatcaggtcaggccgagattagatcaggcttagcatatttgtcctggtcgatcttaccatataccccctccatcccataaaaaattcaatcctaccatgtgtgtccagattcacagtggattagctattttttaatacagagcagttggtggcagaacatgtagtaaaagcaagtataaaacaggcaagtaaaacatgttaggtaaggctagctgacaccctagcagtactatccctattacatgcttagatacaggctggactttagggaaataagtgcttattaattaagctattaacttgtattgttagaacttataaatttagaattctgtaagaaaatttgtaaatagaagtacctcatccttttgatcaaccggctccatgtttctcccaaaaaaggtgttttggctaccagcctcggcattatgggggtcgttgtacttgccatcttcggtgcggtacaggaactccttgggatcaaacagagcaccatgagaactcctcccaacagctagcaggttgtatttttcgaggagggtccggcgggtgttcaggtagagtaaaccgatcggaacaggcaccttgtgccacaggtttctcttgtcaaatgcatgaatgaactacatgaacgaaattaacaagaattaattagttaatacattggaaatttaattaggtatatgtgcatatgtaaaattaaatacttcaactaacgaggaatatcaattggaaagcttcaaataacttgaatagttaataaggtggtgcacaagcatgatcaaacttaagtatatatgcgtgcttctataatggccatatttacgaacataatttgttaagtaccaggatatgcagggaaaaaaatattacatattgacacacatactcatttgaaaatatacttaattataacggattgccctaattaaatggtgcatcaaacttaatttggcgtattttttgatatatagacaacacacacacacacacctgagatattaattaaaacagatcaaacaattaaaatcctgcatcgctagctcaaaatataatttattttttgttgcattagaattgttatatatattatatgtgatcaattattttattataaaaataacacttcattaatgccaagatgtaattaactgaagtgctcacctatatatgtatgcatcctctctaattaacacccttatcttgctataaaataaaaagatgaccacatcaattacatgcatactgattaattttcatagataataacataatccatatatatgatcagacattaatatataatcactacctgatattcttaaggaataggtatgaatgtatattgtatgcagtatgtacatcaaataaatacttgatatataaattactcttaaaaattaattaaaacgtactagaaagccaatcttgtcaaagaaagacatcttagagaaaacatcacggaggtccggatgaacacgaggcttgaagagtcctgaacccat

SEQ ID NO: 2 - DNA, 3063 bp, Oryza sativa
atgtgcatgattgatttgttggtaagttgcgtactttataggcatttaaataaataaaaaagatatgatgtgtaggtgtagctgctcaggaccgataattttatgggagctgatacagtgataccgtggctgtgtttagtttctgaaattggggagaagtttgggaaaagttggtagtttagaaaaagagttgagagtttatgtgtgtaggaaagttttggatgtgatgtgatgtgatgaaaagttgggagttggggaaagtttggtgtgaactaaacacatccacggaccggtcaaacttgtttatatgtgttgtagttccaatcgttgatacggcatgtaaatttgtttattatgcagcgaaaatttcttattatgcatataaaaccaatttgttgagccaaattgttaaatatgtgatcagaatttaagcaatcaatatgaccggattagttttctactaggattttgtattataggattagtttcctactaggacttcgagtctttgcctattaggactcctagattctcttatatgtattccttgtaagccgcacaggaagaggtgcgacagtgcaatatatccctgcgtttgtatccgactcctaaatagtgatcattgccagttggcgtccgtggtttttttccgacgtaaaattccgtgtctgcgttatgcatctattttcttaacgagtggtatcagagcattggagattcgatctacacgacgtggtcgtttttctttctgcaatccctgccaccggtgcaatttctcggatcgaagagtcgatctaattagcgacggcggtagctatgtcgtcattagtctcatctagtcagcaagacaaaggtggagtctatctgtgaggcgtcaatgccgggcacagattaattaggtttaataaattcgtctcgcggtttacagacggaatctgcaattagtttttttattagtatgcgaaaactccataaaacattcgatgtgacaccccaaaactttatacccctataactaaggcctggtttagttcccaaaatttttttcccaaaaagtcacatcggatctttggatatatacatcaagtattaaatatagatttaaaaaaactaattacacagtatgcatggaaatcatgaaacgaatcttttgagcctaattagtctataattaaccataagtactacagtaaccaacatatgctagtgacagattaattaagctcaaaatattcgtctcgtggtttccaggcgagttatcaaattagatttttcattcgtgacctgtaaccccttccgatatctgaccaaatgtccaatgtaacaaccaaaaattttcttttcgcgaacctaattaaacaccccaaccaaaaaacctcacatcgcattcagcacgaataaatacggtcggaaggacaaggtgacgagaaaactcgtggccaaaggacggcctgaataaatacgaataaatccagctgcagctcaaataatatggaggaggctttgatacataagtacaattgtgggttgtgggtaccctcaatgatggtgtacccttcctcccataataattaagtacagttatggtttcagtgccgaactttaaccacttattttatttgaaatatttttacgattaatatttttattgttattagtatttttattgttattagatgataaaatatgaatagtgcttttatacttgacttaactttaaaatatttttttataaattttttaaataagattgacagtaaaatgttggagacgaaaacttactgatgaactttataatgtgacggagagagtactattcgtaggacagtgatggagtacgacgcacgcagctgcatacgcgccaagtggacaaggaagaaattcagacatcgacggcgacactgacagcaaaatcgtgggttgtggtcgttgcagctggacagggttacacgtctcttgtgcatcggcctgcgattggcgagaccctctcaactgtggatatagctacatccctgcatgcatcatcattttgcttgtgcatttgcgtgcacgtgcccttcgtaccaggaaatcaattccggtgcattgatttggttctgtttaattatgtgaatgcaaattcacgttcaattgcaagccatgtttacgaggaggtttatacggagatcaactttattttatacatccagagtatttctactaggattccataatttataccagtagatttaggattttattcccagtacaagtttaaagtttaatctatcgctcctggattgagatgctaatattctcgtttgaagtttgatctatcgctcctgaattgagatgcgaatattctcgttacggcagacaatggagactaagaggaaccgaggaagaagaataacaaacactgcatgatcgatacaggtgatcgatggaacaaaacattgtttttccattgatcaacaaatttaattgccctttccctaaaaataggtcacctatgtcagtctatggccgtgtttagttcgtgtgccaaaaaatttttagtataaggacacatatttgaagtattaaacttaaactaataacaaaacaaattacagattctgcctgtaaactgcaagacgaatttattaagcctaattaatccgtcattagcaaatatttactgtagcatcacattgttaaatcatggcataattagacttaaaagattcgtctcgcaatttacatgtaaactgtataattggttttttcatccatatttaatattttatacatatatatgtctaaatatttgatgttagacgtttgaagggaattaaacactgcctatgtgtaaaggctaaggaagaaggaggcctgactccatcaggagtacagtccggcgtcatacgtggttgccatactaagaggcaatccacactggccattgggaagccaaggaagaaccacataacagttggtcgatcagaaattgcaaaaatcacaagaagaattattccgctgcagcagtttattgaggaccagatctcgggtctaaattttacgtgcatcgaacccttttattag

SEQ ID NO: 3 - DNA, 1189 bp, Oryza sativa
atgggtagtctgtgtggcaccatgattatcatcttggccatgctgccagcaatcctaactatggctgatccatactgtgactgtgattgtccccagcaatgtgaagttaagttgcactattacttgcaccagttccgtgctggagcagatcatccaaaccgcaatgaagaattcgtgacaagtggaggaccttctgggttaggcgctgggcttattcatgactggagtttaaccactggacttgaccccaatgtaaatattgttggacgtgcacaaggatggcatatcgttgccagccaaagttccccagctaattggtacctttctcagaatatagtatttcaggacaggtaaatgctactcacattttagatgggcaatatactgcttttccattggcttgtatcactaaattgcaccatctccatggtaccattatagtaagtatgcagggtctacacttcaggtgatgggtatcattgaaggaagtgaagagaaagttggagagtggtctattatgggtgggactggtgaatttactaatgcgcgtggtaatatcaagtatagagctatcaagaaggaagatgttgagtggataagggaacttgatattcaggtgctctacactccgaacactccatcagatgtaagttttttattaagtactgctacaactcttttacaggttgtttggttggaacagtgggatatatgatggtctacaaagttattgttatatgcatatttcataaatatttgtactagctattgataaagttcacaagacaaaagaaaatacatgaatggatgttaggaagaaaaactatattttctgaaagctgatcacccaagttgagctgatagctaggccagtaccgggccatgaaactttttgaataatggtaactcgatcaacagagaaacctcgatcagtacatgtaccataagaattttgctttctttcaggacgatcaatttaagttaaaatttcaaaacccctactaccaatcttgtttttgcatgaacatatatcacataaagcaaagctttcattgagtgattgtttactttcacttaatctgttggtgaaaagctgttactgtcctgctatttatattaaccattatacactaacacctttttcattgtaaaggttcaagttgcaaagaatattacgaaggggaactaa

SEQ ID NO: 4 - DNA, 3356 bp, Oryza sativa
ttaattcttcttcttttggtccacaagctgctgcgcttctttcacgagggaagcgagagtactgattggcttcaatgagcccagattacgaatgacttgaaatacctttcctctgtaataaccactggtgagaaacatagagccgtcgatgtcagacatggtcttggaagcaagtgacagagactgcatcaagctctgcaagtcttcatccacatagctgccttcattttgcatcattgatatgaccatcttagtggtgtgttgagctattctaaggctgtcgaccttagggtcattgtttgtttctacaatctccttgagcttcctccaaacgctaaatttatcaactccatggcaagtcgcatagaactgttgattcagatcttgatatgaaactgtgaatttgtcacagatagtcacacagagctgtaacaagaattggatgcattggctcttcatattgaaatttggtacgtgagatgacaaactgtcattatcttgtaaataatcacttgggttttcgatgtcggtttcttgtgctactatggacctaggttccgtttctgtttgcagttcttgtcctgttggcctacaatgaaggacttctgcaagaacctgagatggtaaggacttttaatctgacagtggtaataaatctaggcaggatcaggccaatttacatatgatgctaatcaaaaggcagcaggtctggagggaacttacctttggcattgccttggtcatcactcccttcaggttctggagacattcatcgtcttcggtgtaattggcacatagacgctctagaattactgctgtagctactctgaacaatatgatgttgtcacggtctaggagaatttttacgagattatcaacagagttattgtttgctttcatgatgatcatggcactaccttccattttggaagaaagcatcaccagtgcttcagctgctggaccaatacccctgctacaatcactgaacgcgcatagcagcatctggacgaattcttccctgcttgcgcaatccatacctgaatccacaatcgccagtgcagcgaggatctccatggctagcatttgcagcttctcgccgcattggtcacactcgagaatagcttccatggcgcttattgctttcttgttgcctaacatttcatggcgcaggtcggtgccagcttccccgggagcgtccacgagcaggttcataacttgcagagacttggacattatcttggaccaagcgccatggccgacgctgtgaagtatgtccgagctgacgggcgcaatgagcttcgcaatcagaccacatgagttggccatggcgctgcggttgacgtcggcggcggcgagggcctcgagaatcaacagaccgaggaacatcatgtctttgcagtgatcatcgtctttctcatcactctcaggcgaaagacggtcaggccgatcatgttcttgatcacatcctctactactcttccatgtctccattaaccattcgcgctcgtacggctccagaaccgtgcttcgaccatactcctccggtagccaggctggtcgccggcgatcatgcgacaacaggcaacattgctcgtatgggtcaagcagggaacaaatgcagtggatccctcgccggaactgctccacccggatctcgccggcgacgtggagcacgatcctcgcagcacactcccggatcctcctgttatatgggcttcttggccccagcgtctcgagcagcctgtgaacaatgtgaccgaacgatggcgatgcagttagctgctgtctgatcagcacaagctgcccaagctgcacacgacgccgcctccggtcgccggagttcgccgacgtctccatgatcggcagcctcaacagcgctcccagtatctccaacccgcggacgtagccgtcacctgacttggactccagcaggtcgacggcgaacgtgaccaggttcctccccgcagcgaaggacgggttcttggcacatccacttctgatttcctgcaaatactgcagaacggcaactccaagatcaggttcattgtattgcagcgacactgcatccactaggctgcctttgaaatttgcaaacgtctccctgtaaccgaaaatcacgccctgcagcagagctagcgtgtacaggacgtccagtgccgattgcttcagattgtcgccgccgtagtcgcgctcgattagccgccacagagaaatcccggcggagatgtacagtccatagaggtagatggcgaacagaagccacattacgatgctcaacatcgctgcttggatcttcatcattgtgatgtatgcactggggcctgcactgatctctgccctacttctgcagatggcaaggtaggagaacccagtgttgctcaactgcttgttcatgatgacattgaacaccctgttggatggtacggtttgagcagcatacgtaacacatggtttgaaattttggtaatatcggccctcccgtcaagcacaaatctacccaaagtttttttttttttatcaaatatttgtaaatttgctcaaaggttgttcaaattcagtcaaaatatgttaaaatttcaaataatttcggccataaaaatcaggaaatcacgaaatttcaaattttcggcaaccacccaaccgaaattgcaaaccctgatgtaacaagtggaattaaggtagaacgcagacgggttcactattttcaaatgcataatttctgggtactgaactttcagattttctgaggtaactaaaaaaattctaattcgaataacttatgcctaaacttaatttctcataagagcttaaaatttttgcgagaagaacatcctacgggaacatccggtaaacccgaaatctcggaaaattcagtccaaaactgtgaaccctgacaagaacgtcatgttcaaaaatcgatctactcctgtactagttagacaagaacaccatgatttaaaccatgtgtttcgacagatgtgccgtttaactagtggctagtttgaagtctaaataggtaatgggaaaagaaaaatcggtcgtgaggacacttacccaaatacctgaaagagggtgattaccgtgagacaccaaaagtccttcttccccagcatggagacgaaaccgccgaggaggacgaccgtggtccaggtaaccacgaggaaaccaagcccattcacggctattgtcatgtaccccataaggacgccatagctgttgatgtactgcacttccaaccagtcggagtcactgggatctagggatgggagacgccggcgagtgtttcgccgtttacggtgctcggctcgtgcgccacggtcatgcagctgcagccccagctctaccccgttggccat

SEQ ID NO: 5 - DNA, 975 bp, Oryza sativa
atgctgctctgtcaaagggcatcaaagaatgcaccaaggatagaatctttcctacaaaagcaagaaacttcaaacccaaaaagatacactctctctgaagtgaaaagaatgactaaatcttttgctcacaagcttggcagaggtggctttggtactgtttataaaggtagcctgcctgatggccgtgagatagccgtcaagatgctaaaggataccaagggtgatggggaggaattcataaatgaggttgctggcattagtaaaacttctcatatcaatgttgttaaccttctaggtttttcccttcaagggtcaaaaagagctctgatctatgagtacatgcccaatggttcacttgatagatattcttttggcgatagctctgtccaaggagataacaccctgagctgggatagactgttcaatattattgtcgggattgctcgagggctggagtatctccactgtcattgcaacattcgcattgtgcattttgatatcaaacctcaaaacattctactggctcaagatttctgtccaaagatctctgattttggcctgtcaaaattgtgccatctaaaggagagcagaatttcgatcaacggactaagaggaacacctggctacattgcacctgaagtgttttccaggcagtatggatctgccagcagcaaatctgatgtctacagctatggaatggtggtccttgagatggctggtgcaaagaaaaacatcaacgttagtacaggtagtagcagcaaatattttccccaatggttatacgataatttggaccagttttgttgccccacgggcgagattagtagccagaccaccgatcttgtaaggaagatggttgtcgttggtttgtggtgcatacaactcgtacctacagatcgaccgtccatgagagaagtccttgagatgttggaaagcaacggtagggacttaccgttgccaccaaaagggctttga

SEQ ID NO: 6 - protein, 634 aa, Oryza sativa
MGSGLFKPRVHPDLRDVFSKMSFFDKIGFLFIHAFDKRNLWHKVPVPIGLLYLNTRRTLLEKYNLLAVGRSSHGALFDPKEFLYRTEDGKYNDPHNAEAGSQNTFFGRNMEPVDQKDELMSPDPFVVATKLLARREYKDTGKQFNILAAAWIQFMVHDWMDHMEDTGQVGSGQFDFLENVMSGQIEITAPKEVANECPLKSFKFHPTKELPTNSDGIKIGHYNVRTAWWDGSAVYGNNEERAEKLRTYVDGKLVIGDDGLLLHKENGVALSGDIRNSWAGVSILQALFVKEHNAVSDAIKEEHPNLSDEELYRYAKLVTSAVIAKVHTIDWTVELLKTKTMRAAMRANWYGLLGKKIKDTFGHIGGPILGGLVGLKKPNNHGVPYSLTEEFTSVYRMHSLIPSTLKLRDPTGQPDANNSPPYLEDIDIGEMTGLKGEEQLSKIGFEKQALSMGYQACGALELWNYPSFFRNLIPQNLDGTNRSDRIDLAALEVYRDRERSVPRYNEFRRRLFLIPIKSWEDLTSDKDAIETIRAIYGDDVEKLDLLVGLMAEKKIKGFAISETAFNIFILMASRRLEADRFFTSNFNEETYTKKGMQWVKTTEGLRDVINRHYPEITAKWMKSSSAFSVWDADY

SEQ ID NO: 7 - protein, 192 aa, Oryza sativa
MCMIDLLDSDGVRRTQLHTRQVDKEEIQTSTATLTAKSWVVVVAAGQGYTSLVHRPAIGETLSTVDIATSLHASSFCLCICVHVPFVPGNQFRCIDLVLFNYVNANSRSIASHVYEEAKEEGGLTPSGVQSGVIRGCHTKRQSTLAIGKPRKNHITVGRSEIAKITRRIIPLQQFIEDQISGLNFTCIEPFY

SEQ ID NO: 8 - protein, 196 aa, Oryza sativa
MGSLCGTMIIILAMLPAILTMADPYCDCDCPQQCEVKLHYYLHQFRAGADHPNRNEEFVTSGGPSGLGAGLIHDWSLTTGLDPNVNIVGRAQGWHIVASQSSPANWYLSQNIVFQDSKYAGSTLQVMGIIEGSEEKVGEWSIMGGTGEFTNARGNIKYRAIKKEDVEWIRELDIQVLYTPNTPSDVQVAKNITKGN

SEQ ID NO: 9 - protein, 804 aa, Oryza sativa
MANGVELGLQLHDRGARAEHRKRRNTRRRLPSLDPSDSDWLEVQYINSYGVLMGYMTIAVNGLGFLVVTWTTVVLLGGFVSMLGKKDFWCLTVITLFQVFGVFNVIMNKQLSNTGFSYLAICRSRAEISAGPSAYITMMKIQAAMLSIVMWLLFAIYLYGLYISAGISLWRLIERDYGGDNLKQSALDVLYTLALLQGVIFGYRETFANFKGSLVDAVSLQYNEPDLGVAVLQYLQEIRSGCAKNPSFAAGRNLVTFAVDLLESKSGDGYVRGLEILGALLRLPIMETSANSGDRRRRRVQLGQLVLIRQQLTASPSFGHIVHRLLETLGPRSPYNRRIRECAARIVLHVAGEIRVEQFRRGIHCICSLLDPYEQCCLLSHDRRRPAWLPEEYGRSTVLEPYEREWLMETWKSSRGCDQEHDRPDRLSPESDEKDDDHCKDMMFLGLLILEALAAADVNRSAMANSCGLIAKLIAPVSSDILHSVGHGAWSKIMSKSLQVMNLLVDAPGEAGTDLRHEMLGNKKAISAMEAILECDQCGEKLQMLAMEILAALAIVDSGMDCASREEFVQMLLCAFSDCSRGIGPAAEALVMLSSKMEGSAMIIMKANNNSVDNLVKILLDRDNIILFRVATAVILERLCANYTEDDECLQNLKGVMTKAMPKLCVTICDKFTVSYQDLNQQFYATCHGVDKFSVWRKLKEIVETNNDPKVDSLRIAQHTTKMVISMMQNEGSYVDEDLQSLMQSLSLASKTMSDIDGSMFLTSGYYRGKVFQVIRNLGSLKPISTLASLVKEAQQLVDQKKKN

SEQ ID NO: 10 - protein, 324 aa, Oryza sativa
MLLCQRASKNAPRIESFLQKQETSNPKRYTLSEVKRMTKSFAHKLGRGGFGTVYKGSLPDGREIAVKMLKDTKGDGEEFINEVAGISKTSHINVVNLLGFSLQGSKRALIYEYMPNGSLDRYSFGDSSVQGDNTLSWDRLFNIIVGIARGLEYLHCHCNIRIVHFDIKPQNILLAQDFCPKISDFGLSKLCHLKESRISINGLRGTPGYIAPEVFSRQYGSASSKSDVYSYGMVVLEMAGAKKNINVSTGSSSKYFPQWLYDNLDQFCCPTGEISSQTTDLVRKMVVVGLWCIQLVPTDRPSMREVLEMLESNGRDLPLPPKGL

SEQ ID NO: 11 - DNA, 20 bp, Artificial Sequence
  Description of Artificial Sequence Synthetic primer
tcaagcttgtggtgcacttg

SEQ ID NO: 12 - DNA, 22 bp, Artificial Sequence
  Description of Artificial Sequence Synthetic primer
ctcctcctgaactcattgtacc

SEQ ID NO: 13 - DNA, 20 bp, Artificial Sequence
  Description of Artificial Sequence Synthetic primer
agtacagtccggcgtcatac

SEQ ID NO: 14 - DNA, 20 bp, Artificial Sequence
  Description of Artificial Sequence Synthetic primer
ccgagatctggtcctcaata

SEQ ID NO: 15 - DNA, 20 bp, Artificial Sequence
  Description of Artificial Sequence Synthetic primer
gcacaaggatggcatatcgt

SEQ ID NO: 16 - DNA, 22 bp, Artificial Sequence
  Description of Artificial Sequence Synthetic primer
tcccacccataatagaccactc

SEQ ID NO: 17 - DNA, 22 bp, Artificial Sequence
  Description of Artificial Sequence Synthetic primer
aggtcgacagccttagaatagc

SEQ ID NO: 18 - DNA, 22 bp, Artificial Sequence
  Description of Artificial Sequence Synthetic primer
ctggtgagaaacatagagccgt

SEQ ID NO: 19 - DNA, 22 bp, Artificial Sequence
  Description of Artificial Sequence Synthetic primer
gtttgtggtgcatacaactcgt

SEQ ID NO: 20 - DNA, 22 bp, Artificial Sequence
  Description of Artificial Sequence Synthetic primer
ggttcctcaaaaacagaagatg

SEQ ID NO: 21 - DNA, 20 bp, Artificial Sequence
  Description of Artificial Sequence Synthetic primer
atgataactcgacggatcgc

SEQ ID NO: 22 - DNA, 20 bp, Artificial Sequence
  Description of Artificial Sequence Synthetic primer
cttggatgtggtagccgttt

SEQ ID NO: 23 - DNA, 20 bp, Artificial Sequence
  Description of Artificial Sequence Synthetic primer
ttcgcaagacccttcctcta

SEQ ID NO: 24 - DNA, 20 bp, Artificial Sequence
  Description of Artificial Sequence Synthetic primer
gagtacatgcccaatggttc

SEQ ID NO: 25 - DNA, 22 bp, Artificial Sequence
  Description of Artificial Sequence Synthetic primer
atgctgctctgtcaaagggcat

SEQ ID NO: 26 - DNA, 22 bp, Artificial Sequence
  Description of Artificial Sequence Synthetic primer
caagctcaaagcccttttggtg

SEQ ID NO: 27 - DNA, 20 bp, Artificial Sequence
  Description of Artificial Sequence Synthetic primer
ccagctcagggtgttatctc

SEQ ID NO: 28 - DNA, 20 bp, Artificial Sequence
  Description of Artificial Sequence Synthetic primer
ttcgcaagacccttcctcta

SEQ ID NO: 29 - DNA, 20 bp, Artificial Sequence
  Description of Artificial Sequence Synthetic primer
ggttggcttgtatggagcag

SEQ ID NO: 30 - DNA, 20 bp, Artificial Sequence
  Description of Artificial Sequence Synthetic primer
cttctacacagccatcggtc

SEQ ID NO: 31 - DNA, 20 bp, Artificial Sequence
  Description of Artificial Sequence Synthetic primer
gcaggaaccctgaggagatc

SEQ ID NO: 32 - DNA, 20 bp, Artificial Sequence
  Description of Artificial Sequence Synthetic primer
ttccccctccagtccttgct

SEQ ID NO: 33 - DNA, 20 bp, Artificial Sequence
  Description of Artificial Sequence Synthetic primer
tccgggagaaggtgttcgag

SEQ ID NO: 34 - DNA, 20 bp, Artificial Sequence
  Description of Artificial Sequence Synthetic primer
cctcctctccaccaaccatg

SEQ ID NO: 35 - DNA, 20 bp, Artificial Sequence
  Description of Artificial Sequence Synthetic primer
agtgcggcttctcctagctg

SEQ ID NO: 36 - DNA, 20 bp, Artificial Sequence
  Description of Artificial Sequence Synthetic primer
gtcagcagtggaggagaacg

SEQ ID NO: 37 - DNA, 20 bp, Artificial Sequence
  Description of Artificial Sequence Synthetic primer
tcgctccatgtcctgctgtc

SEQ ID NO: 38 - DNA, 20 bp, Artificial Sequence
  Description of Artificial Sequence Synthetic primer
gctcagtgtccgccaagatc

SEQ ID NO: 39 - DNA, 25 bp, Artificial Sequence
  Description of Artificial Sequence Synthetic primer
gcagtcagtgacatgtttgatcaac

SEQ ID NO: 40 - DNA, 21 bp, Artificial Sequence
  Description of Artificial Sequence Synthetic primer
cttagcactcacatcggagac

SEQ ID NO: 41 - DNA, 20 bp, Artificial Sequence
  Description of Artificial Sequence Synthetic primer
catcgagatgccgttcctgc

SEQ ID NO: 42 - DNA, 20 bp, Artificial Sequence
  Description of Artificial Sequence Synthetic primer
cgtctgcttcagcttcgtcc

SEQ ID NO: 43 - DNA, 21 bp, Artificial Sequence
  Description of Artificial Sequence Synthetic primer
cagtaattttggatatatggg

SEQ ID NO: 44 - DNA, 22 bp, Artificial Sequence
  Description of Artificial Sequence Synthetic primer
taatccgtaacgtttcttgtgc

SEQ ID NO: 45 - DNA, 20 bp, Artificial Sequence
  Description of Artificial Sequence Synthetic primer
cggttactagcgtggtttcg

SEQ ID NO: 46 - DNA, 20 bp, Artificial Sequence
  Description of Artificial Sequence Synthetic primer
ctttcccaccaacgctgatc

SEQ ID NO: 47 - DNA, 20 bp, Artificial Sequence
  Description of Artificial Sequence Synthetic primer
gagtacatgcccaatggttc

SEQ ID NO: 48 - DNA, 20 bp, Artificial Sequence
  Description of Artificial Sequence Synthetic primer
actgcctggaaaacacttca

SEQ ID NO: 49 - DNA, 20 bp, Artificial Sequence
  Description of Artificial Sequence Synthetic primer
ttgctgacaggatgagcaag

SEQ ID NO: 50 - DNA, 121 bp, Oryza sativa
ttttattacaaataacatacaatacactcgatttggcaaatatacctagggctaaatgccggttccataaacccagtggtaaaaacaagccacatgaaccagttagtggcaaaaataagtt

SEQ ID NO: 51 - DNA, 121 bp, Oryza sativa
tcattcctagttgcacggaagtattcgccaaatacttcagtgttaaattgtaacgaatgccagacaacctcatggcgaattacagcggggctaaaaattaatagacgctttaaatagttag

SEQ ID NO: 52 - DNA, 33 bp, Oryza sativa
aaatacgccggccaacatatacttgtccaaatc

SEQ ID NO: 53 - DNA, 33 bp, Oryza sativa
ttggacccacatgtaattgagagaatgagggac

SEQ ID NO: 54 - DNA, 12 bp, Artificial Sequence
  Description of Artificial Sequence Synthetic oligonucleotide
atgatgatgatg

SEQ ID NO: 55 - DNA, 12 bp, Artificial Sequence
  Description of Artificial Sequence Synthetic oligonucleotide
gagagagagaga

SEQ ID NO: 56 - protein, 6 aa, Artificial Sequence
  Description of Artificial Sequence Synthetic 6xHis tag
HHHHHH

SEQ ID NO: 57 - DNA, 21 bp, Artificial Sequence
  Description of Artificial Sequence Synthetic primer
tgagatagccgtcaagatgct

SEQ ID NO: 58 - DNA, 20 bp, Artificial Sequence
  Description of Artificial Sequence Synthetic primer
aaggaccaccattccatagc

SEQ ID NO: 59 - DNA, 23 bp, Artificial Sequence
  Description of Artificial Sequence Synthetic primer
tgagccagtagaatgttttgagg

SEQ ID NO: 60 - DNA, 22 bp, Artificial Sequence
  Description of Artificial Sequence Synthetic primer
ctagagtatctccacagtcgtt

SEQ ID NO: 61 - DNA, 22 bp, Artificial Sequence
  Description of Artificial Sequence Synthetic primer
ctgaagtgaaaagaatgactaa

SEQ ID NO: 62 - DNA, 23 bp, Artificial Sequence
  Description of Artificial Sequence Synthetic primer
tgagccagtagaatgttttgagg

SEQ ID NO: 63 - DNA, 22 bp, Artificial Sequence
  Description of Artificial Sequence Synthetic primer
ccgaagtaagaagaatgacgga

SEQ ID NO: 64 - DNA, 23 bp, Artificial Sequence
  Description of Artificial Sequence Synthetic primer
tgatccaggagaatgttttgtgg

SEQ ID NO: 65 - DNA, 22 bp, Artificial Sequence
  Description of Artificial Sequence Synthetic primer
tccaaagatctctgattttggc

SEQ ID NO: 66 - DNA, 22 bp, Artificial Sequence
  Description of Artificial Sequence Synthetic primer
gctttccaacatctcaaggact

SEQ ID NO: 67 - DNA, 975 bp, Oryza glaberrima
atgctgctctgtcaatgggcatcaaagaatgcaccaaggatagaatctttcctacaaaagcaagaaacctcacacccaaaaagatacactttctccgaagtaagaagaatgacggaatcttttgctcacaagcttggcagaggtggcttcggtactgtttatagaggtagcctgcctaatggccgtgagatagctgttaagatgctaaaggacagcaaaggtgatggggaggaattcataaatgaggttgctagcattagtaaaacttctcatatcaatgttgttaaccttctaggtttttcccttcaagggtcaaaaagagctctgatctatgagtacatgtccaatggttcactcgatagatattcttttggcgataactctgtccaaggagataacaccctgagctgggatagactattcgatattattgtcgggattgctcaggggctagagtatctccacagtcgttgcaacattcgcattgtgcattttgatatcaaaccacaaaacattctcctggatcaagatttctgtccaaagatctctgattttggcctgtcaaaattgtgccatctaaaggagagcagaatttcgatcaacggactaagaggaacacctggctacattgcacctgaagtgttttccaggcagtatggatctgccagcagcaaatctgatgtctacagctatggaatggtggtccttgagatggctggtgcaaagaaaaacatcaacgttagtacaggtagtagcaccaaatattttccccaatggttatacgataatttggaccagttttgttgccccacgggcgatattagtagccagaccaccgatcttgtaaagaagatggttgtcgttggtttgtggtgcatacaactcgtacctacagatcgaccgtccatgagagaagtccttgagatgttggaaagcaacggtagggacttaccgttgccaccaaaagggctttga

SEQ ID NO: 68 - DNA, 20 bp, Artificial Sequence
  Description of Artificial Sequence Synthetic primer
ctggacttgaccccaatgta

SEQ ID NO: 69 - DNA, 20 bp, Artificial Sequence
  Description of Artificial Sequence Synthetic primer
tctgatggagtgttcggagt

(19) United States
(12) Patent Application Publication
(10) Pub. No.: US 2015/0082475 A1
(43) Pub. Date: Mar. 19, 2015

(54) Root Growth, Nutrient Uptake, and Tolerance of Phosphorus Deficiency in Plants and Related Materials and Methods

(71) Applicants: International Rice Research Institute, Metro Manila (PH); Japan International Research Center for Agricultural Sciences, Tsukuba (JP)

(72) Inventors: Sigrid Heuer, Stonyfell (AU); Rico Gamuyao, Nagoya-shi (JP); Joong Hyoun Chin, Los Banos (PH); Matthias Wissuwa, Tsukuba (JP)

(73) Assignees: INTERNATIONAL RICE RESEARCH INSTITUTE, Metro Manila (PH); Japan International Research Center for Agricultural Sciences, Tsukuba (JP)

(21) Appl. No.: 14/263,667

(22) Filed: Apr. 28, 2014

Related U.S. Application Data
(60) Provisional application No. 61/868,981, filed on Aug. 22, 2013.
(60) Provisional application No. 61/816,525, filed on Apr. 26, 2013.

(51) Int. Cl. C12N 15/82; C12Q 1/68; A01H 1/02

(52) U.S. Cl. CPC C12N 15/8216; A01H 1/02; C12Q 1/6895; C12N 15/8209; C12N 15/821; C12N 15/8261; C12N 15/8243; C12Q 2600/13; C12Q 2600/158
USPC 800/267; 800/290; 435/419; 435/412; 800/298; 800/320.2; 800/320.1; 800/320.3; 800/320; 435/6.12; 506/9

(57) ABSTRACT

Described herein are methods and materials useful for improving root growth and nutrient uptake in cereal grasses. In particular, present disclosure provides methods for increasing root growth and nutrient uptake in a cereal grass involving marker assisted selection and backcrossing. The present disclosure also provides recombinant DNA for the generation of transgenic plants, transgenic plant cells, and methods of producing the same. The present disclosure also provides materials and methods useful for improving the tolerance of a cereal grass to phosphorus-deficiency The present disclosure further provides methods for generating transgenic seed that can be used to produce a transgenic plant having increased root growth, nutrient uptake, and phosphorus-deficiency tolerance.

### CROSS REFERENCE TO RELATED APPLICATIONS

This application claims the priority benefit of U.S. Provisional Application No. 61/868,981, filed Aug. 22, 2013, and U.S. Provisional Application No. 61/816,525, filed Apr. 26, 2013.

### STATEMENT REGARDING SEQUENCE LISTING

The Sequence Listing, filed electronically and identified as 1-55191-IRRI-13-003_SL.txt, was created on Apr. 28, 2014, is 52,844 bytes in size and is hereby incorporated by reference.

### BACKGROUND OF THE INVENTION

Cereal grasses, cultivated for their edible seeds, are grown in greater quantities and provide more food energy worldwide than any other type of crop. Cereal grasses comprise a range of crops, including corn, rice, wheat, barley, sorghum, millet, oats, and rye. Together, maize, wheat and rice account for nearly half of all food calories consumed globally. Phosphorus (P) is of unequivocal importance for the production of such food crops and the demand for P-fertilizer is increasing worldwide. The deficiency of phosphorus (P) in soil is a worldwide problem affecting about 50% of the rice-cultivated area. Low P in soil may be due to the low P content of the parental material, low pH and/or soil with high P-fixing characteristics

In Asia, where rice is the main and sometimes only source of calories, 40% of the rice is produced in rain fed systems with no or little water control and frequent occurrence of floods, droughts, and other calamities. In addition, 60% (29 Mha) of the rain fed lowland rice is produced on poor and problem soils (FIG. 1A), which are constrained by a multitude of abiotic stresses and are naturally low in phosphorus or P-fixing. Resulting rice yields are therefore low and, not surprisingly, poverty in these regions is amongst the highest in the world.

In sub-Saharan Africa (SSA), many soils are characterized by deficient levels of plant-available P. Most of the soils in the semiarid zone were derived from acidic parent material that contained low levels of P. For the once P fertile soils, soil P stocks have decreased as a constant population growth has led to continuous cropping on the same land without an adequate fertilization. Annual average nutrient loss in SSA was estimated at 22 kg of nitrogen (N), 2.5 kg of phosphorus (P), and 15 kg of potassium (K) per hectare of cultivated land, which accounted for an annual loss equivalent to US $4 billion in fertilizer. These rates are several times higher than Africa's annual fertilizer consumption, which accounts for only 0.8% (1.29 Mt) of the global fertilizer consumption.

Although P deficiency in soil could be alleviated through fertilizer application, the increasing price of fertilizer is becoming further prohibitive for resource-poor farmers in small scale farming systems. The situation will be further aggravated given that phosphate rock, the source of P-fertilizer, is a finite and non-renewable resource that is concentrated in only a few countries (Morocco, China, USA), and mining costs are rising. Apart from the need for long-term strategies to address this problem, the development of rice varieties with high productivity under low-P and other stress conditions is a valid and necessary approach to improve yield and enhance food security in rice-dependent countries.

In recent years, a specific group of rice (aus-type varieties) that originates from South Asia from regions with poor and problem soils, mainly in India and Bangladesh, (S. M. Haefele & R. J. Hijmans, Proceeding of the 26^{th} International Rice Research Conference, pp. 297-308, 2007; S. M. Haefele & R. J. Hijmans, Rice Today, Vol. 8, pp. 30-31, 2009; J. P. Londo et al., Proc Natl Acad Sci, Vol. 103, pp. 9578-9583, 2006) (See FIG. 1A) has been recognized as a valuable source of tolerance genes. For instance, the donor of the submergence-tolerance gene SUB1A is an aus-type variety and rice breeding lines with this gene (Sub1 or “scuba” rice) survive up to two weeks in flooded fields (K. Xu et al., Nature, Vol. 442, pp. 705-708, 2006; D. O. Manzanilla et al., Agricultural Systems, Vol 104, pp. 335-347, 2011). Likewise, tolerance of salinity and heat (R. Wassmann et al., Adv Agron, Vol. 101, pp. 59-122, 2009) and other stresses is present in such varieties.

The aus-type variety Kasalath, which is tolerant of P-deficiency, was identified about a decade ago and a major quantitative trait locus (QTL) associated with tolerance was identified (M. Wissuwa et al., Theor Appl Genet, Vol. 105, pp. 890-897, 2002). Currently, phosphorus uptake 1 (Pup1) is the only P-related QTL available for marker-assisted breeding programs and tolerant Pup1 breeding lines have proven effective in field trials (J. H. Chin et al., Theor Appl Genet, Vol. 102, pp. 1073-1086, 2010; J. H. Chin et al, Plant Physiol, Vol. 156, pp. 1202-1216, 2011) (FIG. 1A). Previous efforts to link Pup1 with known P uptake-related mechanisms showed that Pup1 near-isogenic lines (NILs) had improved root growth under stress but the underlying mechanisms remained enigmatic (M. Wissuwa, Plant and Soil, Vol 269, pp. 57-68, 2005). This indicates that Pup1 might act via a novel mechanism or that the underlying gene may be missing in the reference genome. Sequencing of the Pup1 locus in Kasalath revealed the presence of an ˜90-kb transposon-rich insertion-deletion (INDEL) that is absent from the Nipponbare reference genome and other intolerant rice varieties (S. Heuer et al., Plant Biotechnol J, Vol. 7, pp. 456-471, 2009) (FIG. 1B).

As rice cultivation expands in area, adaptations to low soil fertility will become increasingly important, particularly with the combination of widespread occurrence of poor soils and low fertilizer application rates. Developing rice cultivars with enhanced P efficiency would represent a sustainable strategy to improve the livelihood of resource-poor farmers. It would therefore be beneficial to identify genes within the Pup1 region that are closely associated with tolerance of P-deficiency and that are highly conserved in stress-adapted rice accessions.

### SUMMARY OF THE INVENTION

Described herein are methods and materials useful for improving root growth and nutrient uptake in cereal grasses. In particular, present disclosure provides methods for increasing root growth and nutrient uptake in a cereal grass involving marker assisted selection and backcrossing. The present disclosure also provides recombinant DNA for the generation of transgenic plants, transgenic plant cells, and methods of producing the same. The present disclosure also provides materials and methods useful for improving the tolerance of a cereal grass to phosphorus-deficiency The present disclosure further provides methods for generating transgenic seed that can be used to produce a transgenic plant having increased root growth, nutrient uptake, and phosphorus-deficiency tolerance.

In a particular embodiment described herein is a method of improving root growth and nutrient uptake in a cereal grass comprising: a) crossing a crossing plant of one variety of cereal grass having chromosomal DNA that includes the polynucleotide sequence of SEQ ID NO: 3 (OsPupK20-2), SEQ ID NO: 5 (OsPSTOL1), or both, with a recipient plant of a distinct variety of cereal grass having chromosomal DNA that does not include the polynucleotide sequence of SEQ ID NO: 3 (OsPupK20-2), SEQ ID NO: 5 (OsPSTOL1), or both; and b)selecting one or more progeny plants having chromosomal DNA that includes the polynucleotide sequence of SEQ ID NO: 3 (OsPupK20-2), SEQ ID NO: 5 (OsPSTOL1), or both.

In another particular embodiment provided herein, the method of improving root growth and nutrient uptake in a cereal grass further comprises the steps: a) backcrossing the one or more selected progeny plants to produce backcross progeny plants; and b) selecting one or more backcross progeny plants having chromosomal DNA that includes the polynucleotide sequence of SEQ ID NO: 3 (OsPupK20-2), SEQ ID NO: 5 (OsPSTOL1), or both.

In another particular embodiment provided herein, the method of improving root growth and nutrient uptake in a cereal grass further comprises repeating the steps of backcrossing the one or more selected progeny plants to produce backcross progeny plants and selecting one or more backcross progeny plants having chromosomal DNA that includes the polynucleotide sequence of SEQ ID NO: 3 (OsPupK20-2), SEQ ID NO: 5 (OsPSTOL1), or both one or more times to produce third or higher backcross progeny plants having chromosomal DNA that includes the polynucleotide sequence of SEQ ID NO: 3 (OsPupK20-2), SEQ ID NO: 5 (OsPSTOL1), or both, and the physiological and morphological characteristics of the recipient plant.

In another particular embodiment provided herein, the selected one or more progeny plants has increased booth growth relative to a control plant

In another particular embodiment provided herein, the selected one or more progeny plants has increased booth growth relative to a control plant in both high- and low-phosphorus conditions.

In another particular embodiment provided herein, the selected one or more progeny plants has improved tolerance to phosphorus-deficiency relative to a control plant.

In another particular embodiment provided herein, the selected one or more progeny plants has increased uptake of one or more nutrients selected from the group consisting of: nitrogen; potassium; and phosphorus, relative to a control plant.

In another particular embodiment provided herein, the cereal grass is selected from the group consisting of: rice; corn; wheat; barley; sorghum; millet; oats; and rye.

In another particular embodiment provided herein, the cereal grass is rice.

In another particular embodiment provided herein, the cereal grass is corn.

In another particular embodiment provided herein, the crossing plant is a rice plant selected from the group consisting of: Kasalath; AUS 196; IRAT 77; Azucena; Pratao Precoce; Apo; Vary Lava 701; AUS 257; Dular; IAC 25; IAC 47; UPL R17; UPL RI 5; Vandana; and Way Rarem.

In another particular embodiment provided herein, the crossing plant is a rice plant of variety Kasalath.

In another particular embodiment provided herein, the recipient plant is selected from the group consisting of: IR 64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

In another particular embodiment provided herein, detection of SEQ ID NO: 5 (OsPSTOL1), or lack thereof is performed using one or more markers selected from the group consisting of: K46-1; K46-K1; K46-CG1; K46-K2; K46-CG2; and K46-3.

In another particular embodiment provided herein, detection of SEQ ID NO: 5 (OsPSTOL1), or lack thereof is performed using marker K46-K1.

In another particular embodiment provided herein, detection of SEQ ID NO: 3 (OsPupK20-2), or lack thereof is performed using forward primer SEQ ID NO: 68 and reverse primer SEQ ID NO: 69.

In a particular embodiment described herein is a method for selecting a cereal grass plant having improved root growth and nutrient uptake relative to a control cereal grass plant, comprising: a) inducing expression or increasing expression in a cereal grass plant at least one polynucleotide encoding at least one polypeptide having at least 70% sequence identity to an amino acid sequence selected from the group comprising: SEQ ID NO: 8 (OsPupK20-2); and SEQ ID NO:10 (OsPSTOL1); and b) selecting a cereal grass plant having improved root growth and nutrient uptake relative to a control cereal grass plant, wherein the induced or increased expression of the at least one polypeptide is obtained by transforming and expressing in the cereal grass plant the at least one polypeptide.

In another particular embodiment provided herein, the selected cereal grass plant, in addition to improved root growth and nutrient uptake, has improved tolerance to phosphorus-deficiency.

In another particular embodiment provided herein, the induced or increased expression of the at least one polypeptide is a result of introducing and expressing the at least one polypeptide in the cereal grass plant under control of at least one promoter functional in plants.

In another particular embodiment provided herein, the at least one promoter and the at least one polypeptide are operably linked.

In another particular embodiment provided herein, the at least one polynucleotide encodes a polypeptide sequence having an identity selected from the group consisting of: at least 70% to SEQ ID NO: 8 (OsPupK20-2); at least 70% to SEQ ID NO: 10 (OsPSTOL1); at least 75% to SEQ ID NO: 8 (OsPupK20-2); at least 75% to SEQ ID NO: 10 (OsPSTOL1); at least 80% to SEQ ID NO: 8 (OsPupK20-2); at least 80% to SEQ ID NO: 10 (OsPSTOL1); at least 85% to SEQ ID NO: 8 (OsPupK20-2); at least 85% to SEQ ID NO: 10 (OsPSTOL1); at least 90% to SEQ ID NO: 8 (OsPupK20-2); at least 90% to SEQ ID NO: 10 (OsPSTOL1); at least 95% to SEQ ID NO: 8 (OsPupK20-2); at least 95% to SEQ ID NO: 10 (OsPSTOL1); at least 96% to SEQ ID NO: 8 (OsPupK20-2); at least 96% to SEQ ID NO: 10 (OsPSTOL1); at least 97% to SEQ ID NO: 8 (OsPupK20-2); at least 97% to SEQ ID NO: 10 (OsPSTOL1); at least 98% to SEQ ID NO: 8 (OsPupK20-2); at least 98% to SEQ ID NO: 10 (OsPSTOL1); at least 99% to SEQ ID NO: 8 (OsPupK20-2); at least 99% to SEQ ID NO: 10 (OsPSTOL1); at least 100% to SEQ ID NO: 8 (OsPupK20-2); and at least 100% to SEQ ID NO: 10 (OsPSTOL1).

In another particular embodiment provided herein, the at least one polynucleotide has a sequence identity selected from the group consisting of: at least 70% to SEQ ID NO: 3 (OsPupK20-2); at least 70% to SEQ ID NO: 5 (OsPSTOL1); at least 75% to SEQ ID NO: 3 (OsPupK20-2); at least 75% to SEQ ID NO: 5 (OsPSTOL1); at least 80% to SEQ ID NO: 3 (OsPupK20-2); at least 80% to SEQ ID NO: 5 (OsPSTOL1); at least 85% to SEQ ID NO: 3 (OsPupK20-2); at least 85% to SEQ ID NO: 5 (OsPSTOL1); at least 90% to SEQ ID NO: 3 (OsPupK20-2); at least 90% to SEQ ID NO: 5 (OsPSTOL1); at least 95% to SEQ ID NO: 3 (OsPupK20-2); at least 95% to SEQ ID NO: 5 (OsPSTOL1); at least 96% to SEQ ID NO: 3 (OsPupK20-2); at least 96% to SEQ ID NO: 5 (OsPSTOL1); at least 97% to SEQ ID NO: 3 (OsPupK20-2); at least 97% to SEQ ID NO: 5 (OsPSTOL1); at least 98% to SEQ ID NO: 3 (OsPupK20-2); at least 98% to SEQ ID NO: 5 (OsPSTOL1); at least 99% to SEQ ID NO: 3 (OsPupK20-2); at least 99% to SEQ ID NO: 5 (OsPSTOL1); at least 100% to SEQ ID NO: 3 (OsPupK20-2); and at least 100% to SEQ ID NO: 5 (OsPSTOL1).

In a particular embodiment described herein is a method for making a cereal grass plant having improved root growth and nutrient uptake relative to a control cereal grass plant comprising: a) transforming a cereal grass plant cell, cereal grass plant, or part thereof with a construct comprising: (1) a polynucleotide encoding a polypeptide having at least 70% sequence identity to an amino acid sequence selected from the group comprising: SEQ ID NO: 8 (OsPupK20-2); and SEQ ID NO:10 (OsPSTOL1); (2) a promoter operably linked to the polynucleotide; and (3) a transcription termination sequence; and b) expressing the construct in a cereal grass plant cell, cereal grass plant, or part thereof.

In another particular embodiment provided herein, the method for making a cereal grass plant having enhanced tolerance of phosphorus-deficiency relative to a control cereal grass plant further comprises a step of selecting for a cereal grass plant having e improved root growth relative to a control cereal grass plant.

In another particular embodiment provided herein, the method for making a cereal grass plant having enhanced tolerance of phosphorus-deficiency relative to a control cereal grass plant further comprises a step of selecting for a cereal grass plant having increased uptake of one or more nutrients selected from the group consisting of: nitrogen; potassium; and phosphorus, relative to a control plant.

In another particular embodiment provided herein, the method for making a cereal grass plant having enhanced tolerance of phosphorus-deficiency relative to a control cereal grass plant further comprises a step of selecting for a cereal grass plant having improved tolerance of phosphorus-deficiency relative to a control cereal grass plant.

In another particular embodiment provided herein, the cereal grass plant displays a phenotype comprising one or more characteristics selected from the group consisting of: greater tolerance to soil phosphorus deficiency relative to a control grass plant; greater total root length relative to a control grass plant; greater root surface area relative to a control grass plant; greater total grain weight per plant relative to a control grass plant; early crown root development relative to a control grass plant; increased nutrient uptake relative to a control grass plant; increased nitrogen uptake relative to a control grass plant; increased potassium uptake relative to a control grass plant; increased phosphorus uptake relative to a control grass plant; increased grain yield relative to a control grass plant; and reduced spikelet sterility relative to a control grass plant.

In another particular embodiment provided herein, the cereal grass plant cell, cereal grass plant, or part thereof is selected from the group consisting of: rice; corn; wheat; barley; sorghum; millet; oats; and rye.

In another particular embodiment provided herein, the construct comprises one or more polynucleotides encoding a polypeptide having at least 70% sequence identity to SEQ ID NO: 8 (OsPupK20-2), at least 70% sequence identity to SEQ ID NO:10 (OsPSTOL1), or both.

In another particular embodiment provided herein, the construct comprises one or more polynucleotides having at least 70% sequence identity to SEQ ID NO: 3 (OsPupK20-2), at least 70% sequence identity to SEQ ID NO:10 (OsPSTOL1), or both.

In a particular embodiment described herein is a method for the production of a transgenic cereal grass plant having improved root growth and nutrient uptake relative to a control cereal grass plant comprising: a) transforming and expressing in a cereal grass plant cell at least one polynucleotide encoding at least one polypeptide having at least 70% sequence identity to an amino acid sequence of SEQ ID NO: 8 (OsPupK20-2), SEQ ID NO:10 (OsPSTOL1), or both; and b) cultivating the cereal grass plant cell under conditions promoting plant growth and development, and obtaining transformed plants expressing OsPupK20-2, OsPSTOL1, or both.

In another particular embodiment provided herein, the method for the production of a transgenic cereal grass plant having improved root growth and nutrient uptake relative to a control cereal grass plant further comprises a step of selecting for a cereal grass plant having improved root growth relative to a control cereal grass plant.

In another particular embodiment provided herein, the method for the production of a transgenic cereal grass plant having improved root growth and nutrient uptake relative to a control cereal grass plant further comprises a step of selecting for a cereal grass plant having increased uptake of one or more nutrients selected from the group consisting of: nitrogen; potassium; and phosphorus, relative to a control plant.

In another particular embodiment provided herein, the method for the production of a transgenic cereal grass plant having improved root growth and nutrient uptake relative to a control cereal grass plant further comprises a step of selecting a cereal grass plant having enhanced tolerance of phosphorus-deficiency relative to a control cereal grass plant.

In a particular embodiment described herein is a transgenic plant cell comprising : a) at least one promoter that is functional in plants; and b) at least one polynucleotide encoding a polypeptide sequence at least 70% identical to an amino acid sequence of SEQ ID NO: 8 (OsPupK20-2), SEQ ID NO:10 (OsPSTOL1), or both, wherein the promoter and polynucleotide are operably linked and incorporated into the plant cell chromosomal DNA.

In another particular embodiment provided herein, the type of plant cell is selected from the group consisting of: rice plant cell; corn plant cell; wheat plant cell; barley plant cell; sorghum plant cell; millet plant cell; oats plant cell; and rye plant cell.

In another particular embodiment provided herein, the plant cell is homozygous for the at least one polynucleotide.

In a particular embodiment described herein is a transgenic plant comprising a plurality of transgenic plant cells, wherein the transgenic plant cells comprise: a) at least one promoter that is functional in plants; and b)at least one polynucleotide encoding a polypeptide sequence at least 70% identical to an amino acid sequence of SEQ ID NO: 8 (OsPupK20-2), SEQ ID NO:10 (OsPSTOL1), or both, wherein the promoter and polynucleotide are operably linked and incorporated into the plant cell chromosomal DNA.

In a particular embodiment described herein is a transgenic plant comprising: a)at least one promoter that is functional in plants; and b) at least one polynucleotide encoding a polypeptide sequence at least 70% identical to an amino acid sequence of SEQ ID NO: 8 (OsPupK20-2), SEQ ID NO:10 (OsPSTOL1), or both, wherein the promoter and polynucleotide are operably linked and incorporated into the plant cell chromosomal DNA.

In another particular embodiment provided herein, the transgenic plant is homozygous for the at least one polynucleotide.

In a particular embodiment described herein is a seed of a transgenic plant described herein.

In a particular embodiment described herein is a plant part of a transgenic plant described herein.

In another particular embodiment provided herein, the transgenic plant exhibits a phenotype selected from the group consisting of: greater tolerance to soil phosphorus deficiency relative to a corresponding non-transgenic plant; greater total root length relative to a corresponding non-transgenic plant; greater root surface area relative to a corresponding non-transgenic plant; greater total grain weight per plant relative to a corresponding non-transgenic plant; early crown root development relative to a corresponding non-transgenic plant; increased nutrient uptake relative to a corresponding non-transgenic plant; increased nitrogen uptake relative to a corresponding non-transgenic plant; increased potassium uptake relative to a corresponding non-transgenic plant; increased phosphorus uptake relative to a corresponding non-transgenic plant; increased grain yield relative to a corresponding non-transgenic plant; and reduced spikelet sterility relative to a corresponding non-transgenic plant.

In a particular embodiment described herein is a method for selecting transgenic plants having improved root growth and nutrient uptake relative to a control plant, comprising: a) screening a population of plants for increased phosphorous-deficiency tolerance, wherein plants in the population comprise a transgenic plant cell having recombinant DNA incorporated into its chromosomal DNA wherein said recombinant DNA comprises a promoter that is functional in a plant cell and that is functionally linked to an open reading frame of a polynucleotide encoding a polypeptide sequence at least 70% identical to SEQ ID NO: 8 (OsPupK20-2) and/or SEQ ID NO: 10 (OsPSTOL1), wherein individual plants in said population that comprise the transgenic plant cell exhibit phosphorous-deficiency tolerance at a level the same as or greater than a level of phosphorous-deficiency tolerance in control plants which do not comprise the transgenic plant cell; and b) selecting from said population one or more plants that exhibit phosphorous-deficiency tolerance at a level greater than the level of phosphorous-deficiency tolerance in control plants which do not comprise the transgenic plant cell.

In another particular embodiment provided herein, the method for selecting transgenic plants having improved tolerance of phosphorus-deficiency relative to a control plant further comprises a step of collecting seeds from the one or more plants selected from the population that exhibit phosphorous-deficiency tolerance at a level greater than the level of phosphorous-deficiency tolerance in control plants which do not comprise the transgenic plant cell.

In another particular embodiment provided herein, the method for selecting transgenic plants having improved tolerance of phosphorus-deficiency relative to a control plant further comprises a) verifying that said recombinant DNA is stably integrated in said selected plant; and b) analyzing tissue of said selected plant to determine the expression of a polypeptide having a sequence at least 70% identical to SEQ ID NO: 8 (OsPupK20-2), SEQ ID NO: 10 (OsPSTOL1), or both.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS 1A-1F: (FIG. 1A) Problem soils in Asia and origin of stress-tolerant aus-type rice varieties. Inlay: Breeding lines with and without the tolerant Pup1 locus under P-deficient field conditions. (FIG. 1B) Relative position of Pup1 candidate genes in Kasalath and the Nipponbare reference genome; INDEL: Kasalath-specific insertion-deletion; OsPupK05-1 is part of OsPupK04-1. (FIG. 1C) Semi-quantitative RT-PCR analysis of Pup1 candidate genes in contrasting Nipponbare near-isogenic lines (NILs) +Pup1 and −Pup1 grown in P-deficient soil +/−P-fertilizer; GAPDH: Glyceraldehyde-3-phosphate dehydrogenase. (FIG. 1D) Quantitative RT-PCR analysis of Pup1 genes in roots of NILs (gene expression +P=1). (FIG. 1E) Top panel: Gel stained with Coomassie blue; Bottom panel: Phosphothreonine-specific immunoblot showing that recombinant OsPSTOL1 protein restores phosphorylation of the light-harvesting complex II (LHCII) in the Arabidopsis stn7 stn8 double mutant (lane 4). (FIG. 1F) Semi-quantitative RT-PCR analysis of OsPupK20-2 in IR64 35S::OsPSTOL1 plants and IR74-Pup1 NILs grown in +P hydroponics.

FIGS. 2A-2C: (FIG. 2A) Representative IR64 35S::OsPSTOL1 plants with high (OX high) and low (OX low) transgene expression of independent events and corresponding Null segregants (−) at eight weeks in P-deficient soil (root photos were taken after harvest). (FIG. 2B) Grain weight, P content, and root dry weight (DW) of IR64 transformants and Nulls. (FIG. 2C) Grain weight of Nipponbare transformants and Nulls. Error bars indicate standard error. Significance is indicated by different letters (ANOVA and Tukey's HSD test) and asterisks (paired t test p<0.05). Bar=10 cm.

FIGS. 3A-3E: (FIG. 3A) Total root length and surface area of IR64 35S::OsPSTOL1 plants (OX high; T2) and corresponding Nulls grown in high-P (100 μM) and low-P (10 μM) hydroponics solution for 15 days. (FIG. 3B) Root data of sister NILs with (IR64-Pup1) and without (IR64) Pup1 grown under the same conditions for 21 days. (FIG. 3C) Representative root scans. Error bars indicate standard error. n=number of plants. Significance (*0.05>p≧0.01, **0.01≧p≧0.001, ***0.001>p) was analyzed by paired t test (95%). Bar=1 cm. (FIG. 3D) GUS expression driven by the native OsPSTOL1 promoter in young IR64 seedlings is observed in parenchyma (PC) and outer parenchyma (OP) cells adjacent to the peripheral vascular (PV) cylinder of the coleoptilar node and in crown root primordia (CRP), but not in emerging crown roots (ECR; arrows). (FIG. 3E) GUS staining in older plants (28 days after germination, DAG) is likewise seen in CRP (asterisk) and additionally in cells surrounding vascular bundles (VBs), which are interconnected by nodal vascular anastomoses (arrowheads).

FIGS. 4A-4B: (FIG. 4A) Approximate chromosomal location of genes with constitutively higher (boxed) or lower (all other genes) expression in roots of 35S::OsPSTOL1 transgenics. QTLs for P-deficiency tolerance are indicated in red and green on the chromosomes. Root-related meta-QTLs and QTLs for grain yield under drought are shown as color-coded bars (see legend). Centromeres are indicated in black. (FIG. 4B) qRT-PCR analysis of up-regulated genes in root samples of IR64 transgenics (OX) and Null controls grown in +P hydroponics (J. Bernier et al., Crop Sci, Vol. 47, pp. 507-518, 2007; I. K. Bipmong et al., J Plant Breed Crop Sci, Vol. 60-67, pp. 60-67, 2011; M. S. Gomez et al., Am J Biochem Biotechnol, Vol. 2, pp. 161-169, 2006; J. C. Lanceras et al., Plant Physiol, Vol. 135, pp. 384-399, 2004).

FIG. 5: Based on sequence comparisons of the OsPSTOL1 conserved kinase domain with publicly available protein kinase amino acid sequences, OsPSTOL1 was identified as a member of the LRK10L-2 subfamily. Three distinct groups can be differentiated within this subfamily. OsPSTOL1 belongs to group III. The two most similar Arabidopsis proteins of group III were included for comparison. Proteins marked with an asterisk lack an N-terminal domain and are therefore classified as receptor-like cytoplasmic kinases (RLCKs).

FIGS. 6A-6B: (FIG. 6A) Genomic DNA of Nipponbare 35S::OsPSTOL1 primary transformants (T0) was digested with SacI. (FIG. 6B) Genomic DNA of IR64 35S::OsPSTOL1 T0 plants was digested with XbaI (left) and Sad (right). DNA was blotted on membranes and hybridized with a DIG-labeled hygromycin phosphotransferase gene probe. Independent transgenic lines selected for further evaluations are marked with an asterisk.

FIG. 7: Expression of the OsPSTOL1 transgene was analyzed by semi-quantitative RT-PCR using leaf RNA samples from individual transgenic plants (+) and Null segregants (−) of the five indicated independent lines. Plants were grown in P-deficient soil. The PCR cycle number is indicated. Glyceraldehyde-3-phosphate dehydrogenase (GAPDH) was included as a control. H₂O, water control.

FIG. 8: Additional phenotypic data of 35S::OsPSTOL1 and Nulls with high (OX high) and low (OX low) transgene expression grown in P-deficient soil under stress (−P/dry down) and control (+P/well-watered,) conditions. See detailed description and examples for details. Significance is indicated by different letters (ANOVA and Tukey's HSD test) and asterisks (paired t test p<0.05).

FIG. 9: Total root length and surface area of IR74 near-isogenic lines with (IR74-Pup1) and sister lines without (IR74) the Kasalath Pup1 locus grown for 21 days in hydroponics under high-P (100 μM) and low-P (10 μM) conditions. Roots were scanned using WinRhizo. Error bars indicate standard error. The data were analyzed by paired t test (95%) and significance levels are indicated as *0.05>p≧0.01, **0.01>p≧0.001, ***0.001≧p; no asterisk: not significant.

FIGS. 10A-10H: (FIG. 10A) The rice variety Kasalath was transformed with an RNAi construct and knock-down of OsPSTOL1 was analyzed by semi-quantitative RT-PCR of root RNA samples from representative plants of four transgenic lines (R1-R4; T3 generation) in comparison to Null segregants and Kasalath wild type controls. (FIG. 10B) Plants were grown in −P hydroponics for 12 days and roots were scanned to obtain total root length and surface area. The other data were obtained manually. *Different letters indicate significant difference (Tukey HSD All-Pairwise Comparisons; alpha=0.05). *roots with diameter 2.000<.L.<=5.000. (FIG. 10C) Representative RNAi plants, Kasalath wild type and Null segregants grown in P-deficient soil for 60 days. (FIG. 10D) Dry weight of Kasalath, Null segregants, and RNAi plants at 60 days after sowing. Significance is indicated by different letters (ANOVA and Tukey's HSD test). (FIG. 10E) Crown root emergence in representative RNAi plants from two independent lines (1, 2) and Kasalath grown in hydroponics culture solution at 8 days after germination (DAG). (FIG. 10F) Crown root number at 8 DAG. Significance is indicated by different letters (ANOVA and Tukey's HSD test). (FIG. 10G) OsPSTOL1 expression was determined by semi-quantitative RT-PCR of representative RNAi plants (R) and Kasalath wild type controls (W). (FIG. 10H) Representative RNAi plant (R) and Kasalath (W) at 84 DAG grown in P-deficient soil.

FIG. 11: Eight rice accessions with the OsPSTOL1 gene (Kasalath group) and fourteen accessions without OsPSTOL1 (Nipponbare group) were used to analyze allelic association between OsPSTOL1 and SNP markers located within 1 Mb distance to the putative OsPSTOL1 downstream genes (see Table 1 and FIG. 4 for details on the genes; for some genes SNP markers were not available).

FIGS. 12A-12C: Phenotype of T2 and T3 35S::OsPupK20-2 plants. Four lines overexpressing OsPupK20-2 were compared to Null control plants. Average grain weight (FIG. 12A; p=0, 0.01, and 0.03 for lines 4c, 9a, and 12a, respectively), average panicle number (FIG. 12B; p=0.03, 0.01, 0, and 0.02 for lines 4c, 6a, 9a, and 12a, respectively), and average tiller number (FIG. 12C; p=0.01, 0.05, 0, and 0.01 for lines 4c, 6a, 9a, and 12a, respectively) were determined.

FIG. 13: Phenotype of T2 and T3 35S::OsPupK20-2 plants.

FIG. 14: Root scan analysis of 35S::OsPupK20-2 plants. Plants overexpressing OsPupK20-2 were grown in +P or −P conditions, along with Null controls in hydroponics. Overexpressing plants showed enhanced root growth at 12d after germination.

FIG. 15: Root scan analysis of 35S::OsPupK20-2 plants. Plants overexpressing OsPupK20-2 were grown in +P or −P conditions, along with Null controls in either hydroponics or in soil. Overexpressing plants grown in +P soil for 18d showed greater root length (p=0.01) and root surface area (p=0.04) when compared to Null controls.

FIGS. 16A-16B: Phenotype of T2 and T3 35S::OsPupK20-2 plants. Plants overexpressing OsPupK20-2 were grown in +P or −P conditions, along with Null controls in soil. Overexpressing plants showed enhanced seedling vigor, as demonstrated by longer shoots, in both +P and −P conditions (p=0.02).

FIGS. 17A-17B: Amplification of unspecific bands when PSTOL1-specific marker (K46-1, based on Kasalath sequence) was used for PCR genotyping of upland NERICA varieties and their parents (FIG. 17A); and upland African mega-varieties (FIG. 17B). DMSO was added to improve PCR amplification. Red circles indicate the amplicon of genotypes selected for sequencing.

FIG. 18: Nucleotide sequence alignment of the PSTOL1 allele from Kasalath (O. sativa, ssp. indica) and CG14 (O. glaberrima). Brackets indicate the region sequenced from the amplicons. Polymorphic SnPs are indicated. Squares indicate the location of allele-specific markers for PSTOL1 alleles of Kasalath and CG14 (single black square=K46-K2 and K46-CG2; red squares=K46-K; blue squares=K46-CG; linked black squares=K46-3). Location of PSTOL1 allele in O. Glaberrima: 116753-115779, Oglab12_unplaced142# O. glaberrima unanchored scaffold derived from chr12 pool6 (represented by Oglab12_{—}0135 thru Oglab12_{—}0185). Precise location and orientation is unknown.

FIGS. 19A-19B: PCR amplification for PSTOL1 using Kasalath (O. sativa, ssp. indica) (SEQ ID NO: 5) and CG14 (O. glaberrima) (SEQ ID NO: 67) allele-specific markers (FIG. 19A). The mixture of these two pairs of allele-specific markers resulted in a duplex-PCR genotyping method (FIG. 19B). (N1 NERICA1, N10 NERICA10, N16 NERICA16, W50 WAB56-50, CG CG14, IRAT IRAT216 (IDSA6), W181 WAB181-18, IDSA IDSA 85, IR 12979, W104 WAB56-104, IACIAC165,Nb nipponbare, Kas Kasalath)

FIG. 20: PCR amplification of the marker K46-3 in genomic DNA of Kasalath and CG14 (O. glaberrima). This marker is located in a common region for both alleles (400 bp). K: Kasalath, CG: CG14, Nb: Nipponbare, W18: WAB181-18, W50: WAB56-50, IR64.

FIG. 21: Abundance pattern of PSTOL1 transcript in four rice genotypes grown in water culture or soil under low or high P condition. Specific-allele markers for PSTOL1-Kasalath (O. sativa) or O. glaberrima, and K46-3 (which amplifies a common region in both alleles) were used for RT-PCR. CG CG14, N10 NERICA10, IAC IAC165, Kas Kasalath. Glab* cycles were increased up to 40, and template doubled, only for CG14.

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

Described herein are methods and materials useful for improving tolerance of phosphorus-deficiency in cereal grasses. In particular, the present disclosure provides recombinant DNA for the generation of transgenic plants, transgenic plant cells, and methods of producing the same. The present disclosure further provides methods for generating transgenic seed that can be used to produce a transgenic plant having increased phosphorus-deficiency tolerance, and methods for increasing tolerance of phosphorus-deficiency in a cereal grass involving marker assisted selection and backcrossing.

The present invention provides transgenic plant cells comprising: a) at least one promoter that is functional in plants; and b) at least one polynucleotide encoding a polypeptide sequence at least 70% identical to SEQ ID NO: 8 (OsPupK20-2) and/or SEQ ID NO: 10 (OsPSTOL1); wherein the promoter and polynucleotide are operably linked and incorporated into the plant cell chromosomal DNA.

Also provided are such plant cells, wherein the type of cell is selected from the group consisting of: rice; corn; wheat; barley; sorghum; millet; oats; or rye.

Also provided are such plant cells, wherein the polynucleotide encodes a polypeptide sequence of an identity selected from the group consisting of: at least 70%; at least 75%; at least 80%; at least 85%; at least 90%; at least 95%, at least 96%; at least 97%; at least 98%; at least 99%; and 100%.

Also provided are such plant cells, wherein the polynucleotide comprises SEQ ID NO: 3 (OsPupK20-2), SEQ ID NO: 5 (OsPSTOL1), or both, or a complementary sequence.

Also provided are such plant cells, wherein the plant cell is homozygous for the polynucleotide.

Also provided are transgenic plants comprising a plurality of such plant cells.

The present invention also provides transgenic plants comprising: at least one promoter that is functional in plants; at least one polynucleotide encoding a polypeptide sequence at least 70% identical to SEQ ID NO: 8 (OsPupK20-2), SEQ ID NO: 10 (OsPSTOL1), or both, wherein the promoter and polynucleotide are operably linked and incorporated into the plant cell chromosomal DNA.

Also provided are such transgenic plants, wherein the plant is selected from the group consisting of: rice; corn; wheat; barley; sorghum; millet; oats; and rye.

Also provided are such transgenic plants, wherein the polynucleotide encodes a polypeptide sequence of an identity selected from the group consisting of: at least 70%; at least 75%; at least 80%; at least 85%; at least 90%; at least 95%, at least 96%; at least 97%; at least 98%; at least 99%; and 100%.

Also provided are transgenic plants wherein the polynucleotide comprises SEQ ID NO: 3 (OsPupK20-2), SEQ ID NO: 5 (OsPSTOL1), or both, or a complementary sequence.

Also provided are transgenic plants, wherein the plant is homozygous for the polynucleotide.

Also provided are seeds of a transgenic plant described herein.

Also provided are plant parts of a transgenic plant described herein.

Also provided are transgenic plants, wherein the plant exhibits a phenotype selected from the group consisting of: greater tolerance to soil phosphorus deficiency relative to a corresponding non-transgenic plant; greater total root length relative to a corresponding non-transgenic plant; greater root surface area relative to a corresponding non-transgenic plant; greater total grain weight per plant relative to a corresponding non-transgenic plant; early crown root development relative to a corresponding non-transgenic plant; increased nutrient uptake relative to a corresponding non-transgenic plant; increased nitrogen uptake relative to a corresponding non-transgenic plant; increased potassium uptake relative to a corresponding non-transgenic plant; increased phosphorus uptake relative to a corresponding non-transgenic plant; increased grain yield relative to a corresponding non-transgenic plant; and reduced spikelet sterility relative to a corresponding non-transgenic plant.

The present invention also provides methods for selecting transgenic plants comprising: a) screening a population of plants for increased phosphorous-deficiency tolerance, wherein plants in the population comprise a transgenic plant cell having recombinant DNA incorporated into its chromosomal DNA wherein said recombinant DNA comprises a promoter that is functional in a plant cell and that is functionally linked to an open reading frame of a polynucleotide encoding a polypeptide sequence at least 70% identical to SEQ ID NO: 8 (OsPupK20-2), SEQ ID NO: 10 (OsPSTOL1), or both, or a complementary sequence, wherein individual plants in said population that comprise the transgenic plant cell exhibit phosphorous-deficiency tolerance at a level the same as or greater than a level of phosphorous-deficiency tolerance in control plants which do not comprise the transgenic plant cell; and b) selecting from said population one or more plants that exhibit phosphorous-deficiency tolerance at a level greater than the level of phosphorous-deficiency tolerance in control plants which do not comprise the transgenic plant cell.

Also provided are such methods, which further comprise a step of collecting seeds from the one or more selected plant that exhibit phosphorous-deficiency tolerance at a level greater than the level of phosphorous-deficiency tolerance in control plants which do not comprise the transgenic plant cell.

Also provided are such methods, wherein said method for selecting said transgenic seed further comprises: a) verifying that said recombinant DNA is stably integrated in said selected plant, and b) analyzing tissue of said selected plant to determine the expression of a polypeptide having a sequence of SEQ ID NO: 8 (OsPupK20-2), SEQ ID NO: 10 (OsPSTOL1), or both.

Also provided are such methods, wherein the plant is selected from the group consisting rice; corn; wheat; barley; sorghum; millet; oat; and rye.

Also provided are such methods, wherein said plant is rice.

Also provided are such methods, wherein the plant is a rice variety selected from the group consisting of: IR 64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

The present invention also provides methods of increasing phosphorous-deficiency tolerance in a cereal grass comprising: a) crossing a plant of one variety of cereal grass having chromosomal DNA that includes the nucleotide sequence of SEQ ID NO: 3 (OsPupK20-2and/or SEQ ID NO: 5 (OsPSTOL1), with a recipient plant of a distinct variety of cereal grass having genomic DNA that does not include the nucleotide sequence of SEQ ID NO: 3 (OsPupK20-2and/or SEQ ID NO: 5 (OsPSTOL1); b) selecting one or more progeny plants having chromosomal DNA that includes the nucleotide sequence of SEQ ID NO: 3 (OsPupK20-2and/or SEQ ID NO: 5 (OsPSTOL1); c) backcrossing the selected progeny plants with additional to produce backcross progeny plants; d) selecting one or more backcross progeny plants having chromosomal DNA that includes the nucleotide sequence of SEQ ID NO: 3 (OsPupK20-2) and/or SEQ ID NO: 5 (OsPSTOL1); and e) repeating steps c) and d) one or more times to produce third or higher backcross progeny plants having chromosomal DNA that includes the nucleotide sequence of SEQ ID NO: 3 (OsPupK20-2) and/or SEQ ID NO: 5 (OsPSTOL1) and the physiological and morphological characteristics of the recipient plant.

Also provided such methods, wherein the cereal grass is selected from the group consisting of: rice; corn; wheat; barley; sorghum; millet; oats; and rye.

Also provided such methods, wherein the cereal grass is rice.

Also provided such methods, wherein the crossing plant is selected from the group consisting of Kasalath; Pup1 NILC443; and NIL14-4.

Also provided such methods, wherein the recipient plant is selected from the group consisting of: IR 64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

Also provided are such methods, wherein detection of SEQ ID NO: 5 (OsPSTOL1) or lack thereof is performed using one or more markers selected from the group consisting of: K46-1; K46-K1; K46-CG1; K46-K2; and K46-CG2.

The present invention also provides methods to select a P-deficiency tolerant plant, comprising screening a population of transgenic plants that have been transformed with SEQ ID NO: 8 (OsPupK20-2), SEQ ID NO: 10 (OsPSTOL1), or both, and selecting a P-deficiency tolerant plant.

The present invention also provides methods to cultivate a cereal grass plant, comprising cultivating a seed herein.

Also provided are such methods, wherein cultivating is under at least one phosphorus-deficient condition.

Also provided are such methods, wherein the phosphorus-deficient condition is selected from the group consisting of: drought; poor soil quality; phosphorus-fixing soil; lack of fertilizer; and overplanting.

The present invention also provides methods to cultivate a cereal grass plant, comprising cultivating a plant part herein.

The present invention also provides methods to induce early crown root development in a cereal grass plant, comprising: cultivating a seed herein or plant part herein and inducing early crown root development.

The present invention also provides methods to induce increased root surface area in a cereal grass plant, comprising: cultivating a seed herein or plant part herein, and inducing increased root surface area in the cereal grass plant compared to a similar plant lacking a polypeptide sequence at least 70% identical to SEQ ID NO: 8 (OsPupK20-2) and/or SEQ ID NO: 10 (OsPSTOL1).

The present invention also provides methods to induce increased root dry weight in a cereal grass plant, comprising: cultivating a seed herein or plant part herein, and inducing increased root dry weight in the cereal grass plant compared to a similar plant lacking a polypeptide sequence at least 70% identical to SEQ ID NO: 8 (OsPupK20-2) and/or SEQ ID NO: 10 (OsPSTOL1).

The present invention also provides methods to induce increased nutrient uptake in a cereal grass plant, comprising: cultivating a seed herein or plant part herein, and inducing increased nutrient uptake in the cereal grass plant compared to a similar plant lacking a polypeptide sequence at least 70% identical to SEQ ID NO: 8 (OsPupK20-2) and/or SEQ ID NO: 10 (OsPSTOL1).

Also provided are such methods, wherein the nutrient uptake is selected from the group consisting of: sodium; potassium; and phosphate.

The present invention also provides methods to induce increased grain yield in a cereal grass plant, comprising: cultivating a seed herein or plant part herein, and inducing increased grain yield in the cereal grass plant compared to a similar plant lacking a polypeptide sequence at least 70% identical to SEQ ID NO: 8 (OsPupK20-2) and/or SEQ ID NO: 10 (OsPSTOL1).

Also provided are such methods wherein the yield increase is selected from the group consisting of: at least 20%; at least 30%; at least 40%; at least 50%; at least 60%; at least 70%; and greater than 70%

Also provided are such methods, wherein cultivating is under at least one phosphorus-deficient condition.

The present invention also provides methods to induce OsHOX1 in a cereal grass plant, comprising cultivating a seed herein or plant part herein, and inducing OsHOX1 in the cereal grass plant compared to a similar plant lacking a polypeptide sequence at least 70% identical to SEQ ID NO: 8 (OsPupK20-2) and/or SEQ ID NO: 10 (OsPSTOL1).

The present invention also provides methods to induce root cell differentiation in a cereal grass plant, comprising cultivating a seed herein or plant part herein, and inducing root cell differentiation in the cereal grass plant compared to a similar plant lacking a polypeptide sequence at least 70% identical to SEQ ID NO: 8 (OsPupK20-2) and/or SEQ ID NO: 10 (OsPSTOL1).

The present invention also provides methods to induce OsDOS in a cereal grass plant, comprising cultivating a seed herein or plant part herein, and inducing OsDOS in the cereal grass plant compared to a similar plant lacking a polypeptide sequence at least 70% identical to SEQ ID NO: 8 (OsPupK20-2) and/or SEQ ID NO: 10 (OsPSTOL1).

The present invention also provides methods to affect the expression of one or more genes in a cereal grass plant, comprising cultivating a seed herein or plant herein, and affecting one or more genes in the cereal grass plant compared to a similar plant lacking a polypeptide sequence at least 70% identical to SEQ ID NO: 8 (OsPupK20-2) and/or SEQ ID NO: 10 (OsPSTOL1), wherein the one or more genes are selected from the genes shown in Table 1.

The present invention also provides methods of any of the claims herein, wherein the type of rice is selected from the group consisting of: indica; japonica; aromatic; and glutinous.

Definitions

As used herein a “transgenic plant cell” means a plant cell that is transformed with stably-integrated, non-natural, recombinant DNA, e.g. by Agrobacterium-mediated transformation or by bombardment using microparticles coated with recombinant DNA or other means. A plant cell of this disclosure can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed transgenic plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

“Percent identity” describes the extent to which the sequences of DNA or protein segments are invariant throughout a window of alignment of sequences, for example nucleotide sequences or amino acid sequences. Percent identity is calculated over the aligned length preferably using a local alignment algorithm, such as BLASTp. As used herein, sequences are “aligned” when the alignment produced by BLASTp has a minimal e-value.

As used herein “promoter” means regulatory DNA for initializing transcription. A promoter that is functional in a plant cell is a promoter capable of initiating transcription in plant cells whether or not its origin is a plant cell, e.g. is it well known that Agrobacterium promoters are functional in plant cells. Thus, promoters that are functional in plants include promoter DNA obtained from plants, plant viruses and bacteria such as Agrobacterium and Bradyrhizobium bacteria.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that imparts enhanced P-deficiency tolerance. A control plant is used to identify and select a transgenic plant that has enhanced P-deficiency tolerance. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit, or signal peptides.

Numerous promoters that are active in plant cells have been described. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. These and numerous other promoters that function in plant cells are known to those skilled in the art and available for use in recombinant polynucleotides of the present disclosure to provide for expression of desired genes in transgenic plant cells.

Furthermore, the promoters may be altered to contain multiple “enhancer sequences” to assist in elevating gene expression. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

The term “quantitative trait locus” or “QTL” refers to a polymorphic genetic locus with at least two alleles that reflect differential expression of a continuously distributed phenotypic trait.

The term “associated with” or “associated” in the context of this disclosure refers to, for example, a nucleic acid and a phenotypic trait, that are in linkage disequilibrium, i.e., the nucleic acid and the trait are found together in progeny plants more often than if the nucleic acid and phenotype segregated independently.

The term “marker” or “molecular marker” or “genetic marker” refers to a genetic locus (a “marker focus”) used as a point of reference when identifying genetically linked loci such as a quantitative trait locus (QTL). The term may also refer to nucleic acid sequences complementary to the genomic sequences, such as nucleic acids used as probes or primers. The primers may be complementary to sequences upstream or downstream of the marker sequences. The term can also refer to amplification products associated with the marker. The term can also refer to alleles associated with the markers. Allelic variation associated with a phenotype allows use of the marker to distinguish germplasm on the basis of the sequence.

The term “interval” refers to a continuous linear span of chromosomal DNA with termini defined by and including molecular markers.

The term “crossed” or “cross” in the context of this disclosure means the fusion of gametes via pollination to produce progeny (i.e., cells, seeds or plants). The term encompasses both sexual crosses (the pollination of one plant by another) and selfing (self-pollination, i.e., when the pollen and ovule are from the same plant or from genetically identical plants).

As used herein, the term “phosphorus-deficiency” refers to a soil condition in which phosphorous is not available for absorption by plants. Conditions in which soil may be phosphorus-deficient include, but are not limited to, drought, poor soil quality, phosphorus-fixing soil, lack of fertilizer, and overplanting.

The Protein Kinase Pstol1 Confers Tolerance of Phosphorus Deficiency

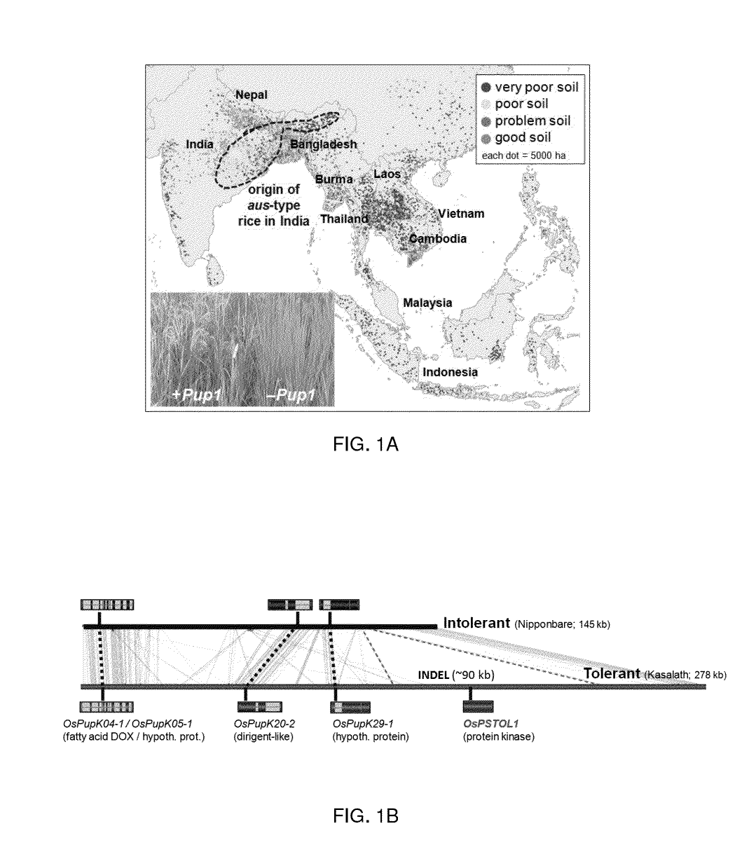

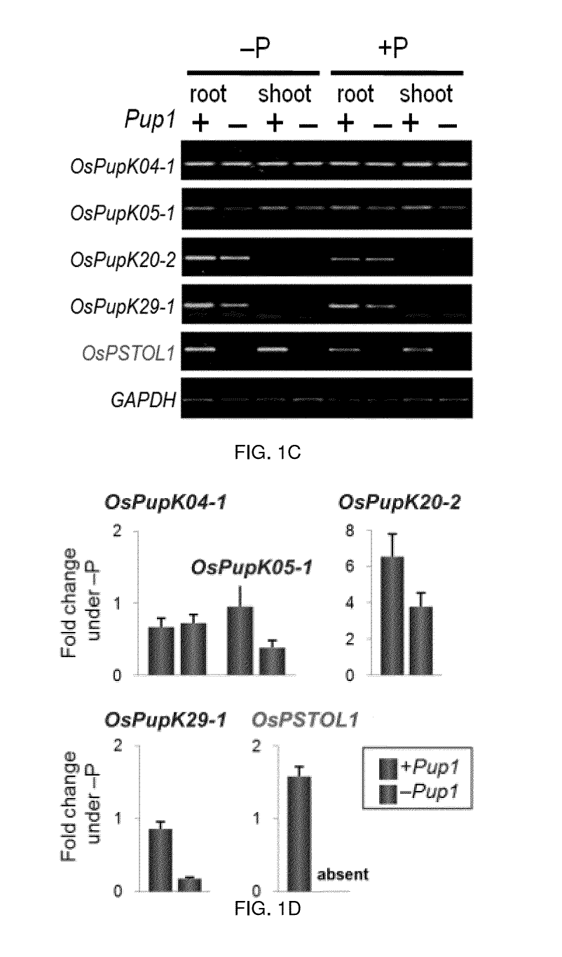

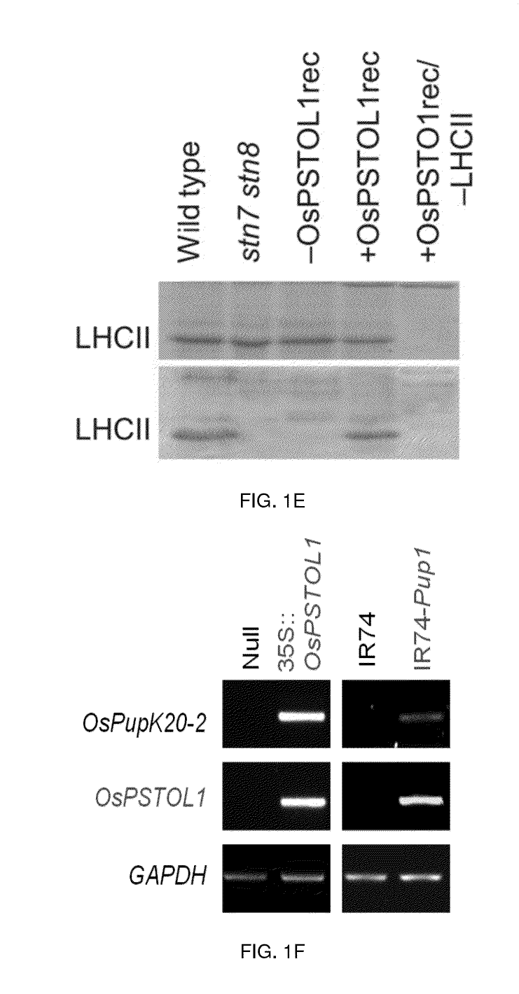

A major genetic determinant of P-deficiency tolerance has been identified, and is described herein. The polypeptide encoded by the gene phosphorus-starvation tolerance 1 (PSTOL1) shows the highest amino acid sequence similarity with serine/threonine receptor-like kinases of the LRK10L-2 subfamily, but lacks the amino-terminal extension typically present in this family. This classifies Pstol1 as a receptor-like cytoplasmic kinase. The protein kinase activity of Pstol1 was confirmed using an in vitro phosphorylation assay using thylakoid membranes isolated from the Arabidopsis thaliana double mutant stn7 stn8, which is defective in STN7 and STN8 (also known as AT1G68830 and AT5G01920, respectively) serine/threonine protein kinases and therefore devoid of phosphorylation of the light harvesting complex II (V. Bonardi et al., Nature, Vol. 437, pp. 1179-1182, 2005). Recombinant Pstol1 protein restored phosphorylation of stn7 stn8 thylakoids to almost wild-type levels (FIG. 1E).

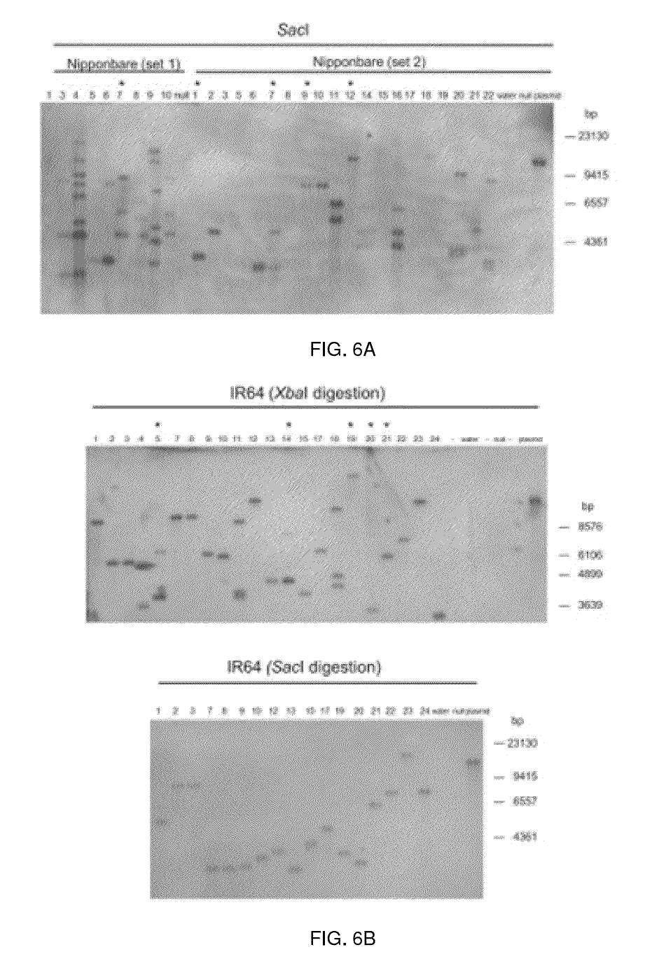

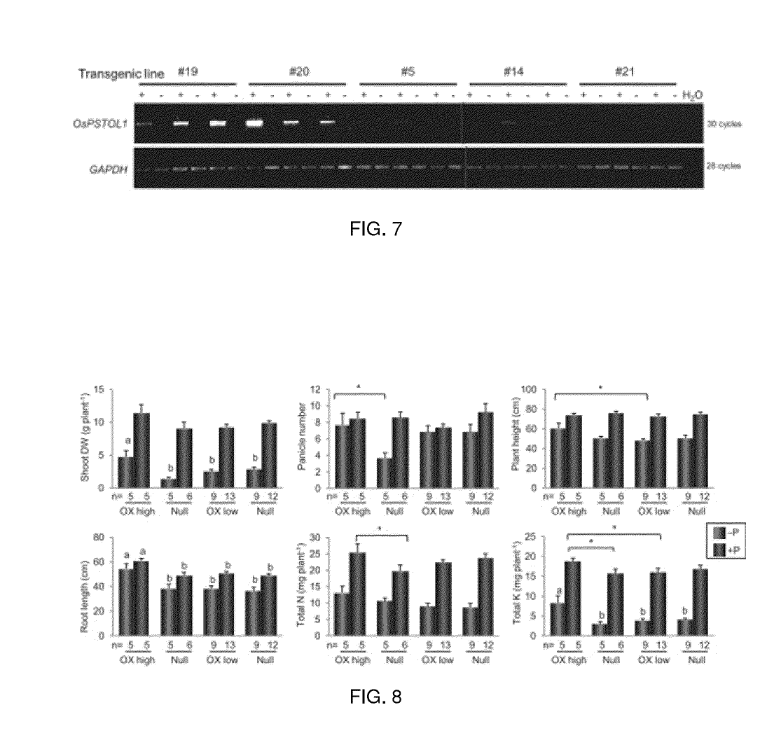

To quantify the effect of OsPSTOL1 on plant performance under low-P stress, transgenic plants were generated with constitutive overexpression (OX) of the full-length OsPSTOL1 coding region (SEQ ID NO 5; 35S::OsPSTOL1). Two rice varieties (IR64, Nipponbare) were used, representing two distinct types of modern irrigated varieties (indica, japonica) that naturally lack the OsPSTOL1 gene (FIG. 6). Other varieties naturally lacking the OsPSTOL1 gene may also be used as OsPSTOL1 recipients, such as PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

A plant from any variety possessing OsPSTOL1 may be used as a donor variety. Donor varieties include, but are not limited to, Kasalath, AUS 196, IRAT 77, Azucena, Pratao Precoce, Apo, Vary Lava 701, AUS 257, Dular, IAC 25, IAC 47, UPL R17, UPL RI 5, Vandana, and Way Rarem. In certain embodiments, the donor variety is Kasalath, Dular, IAC 25, or IAC 47. In yet other embodiments, the donor variety is Kasalath.

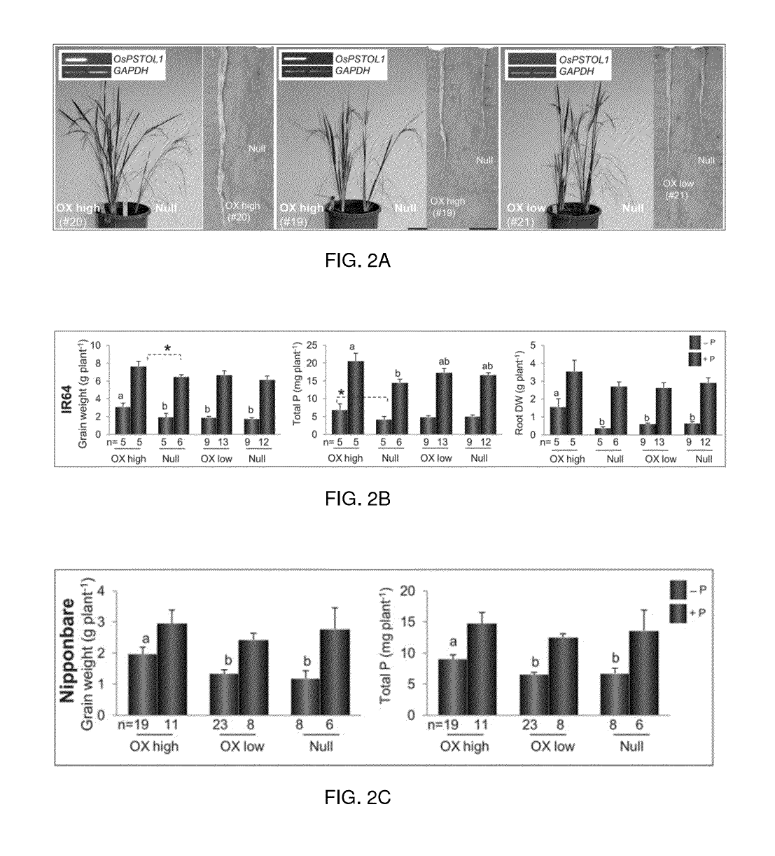

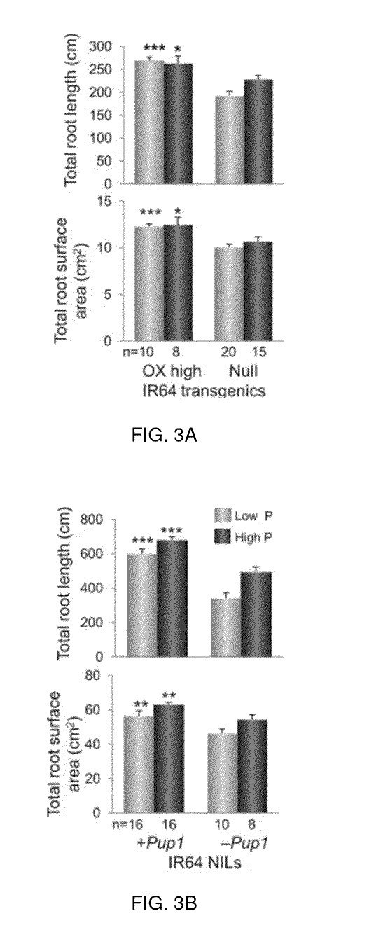

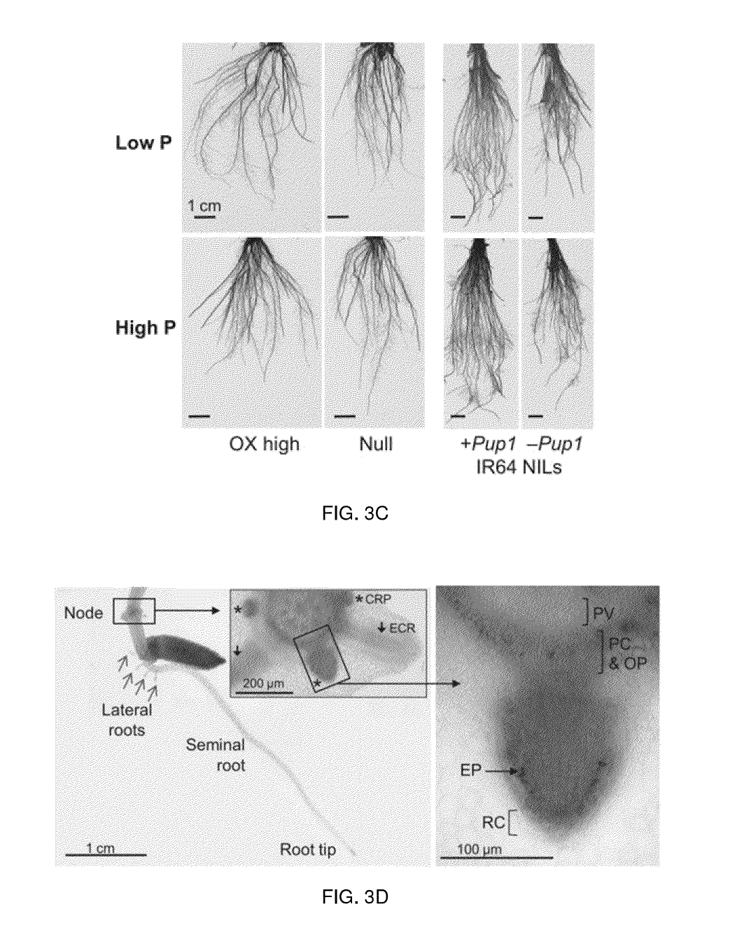

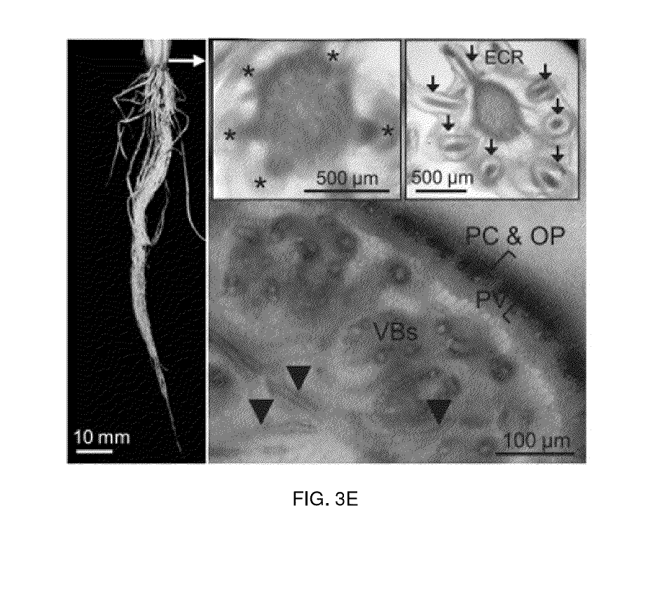

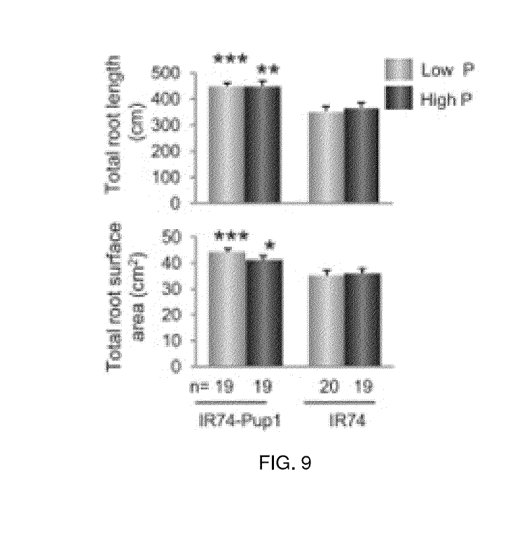

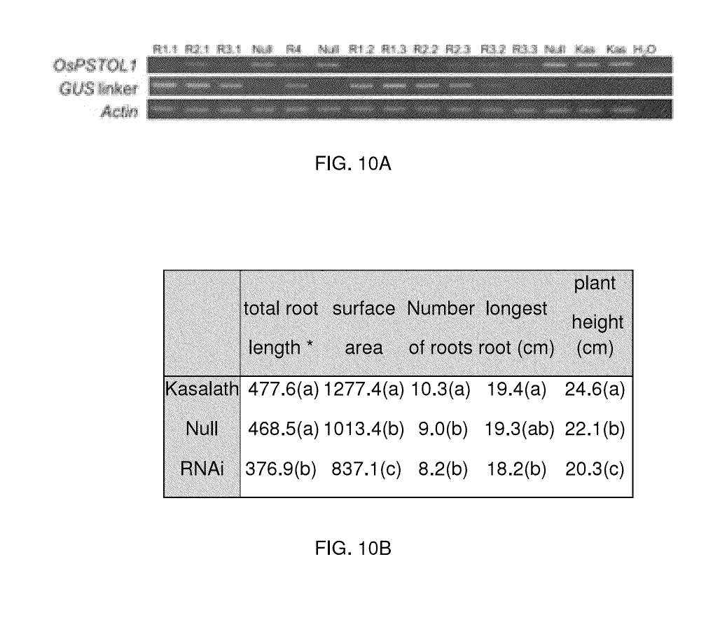

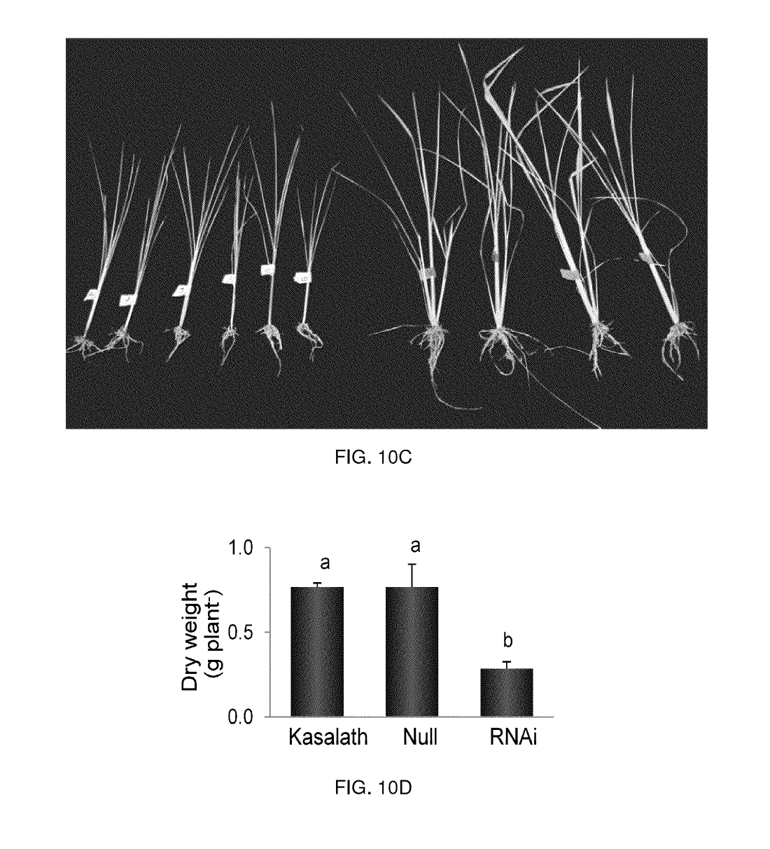

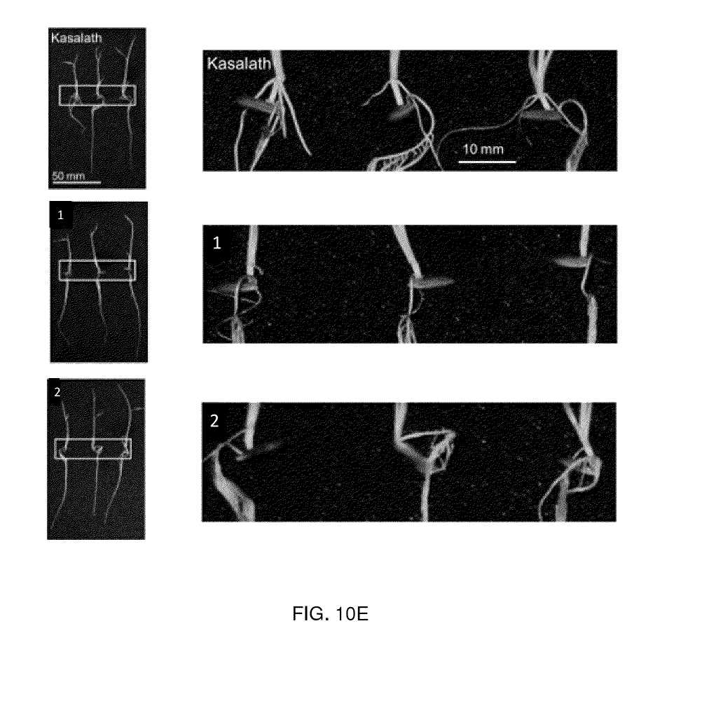

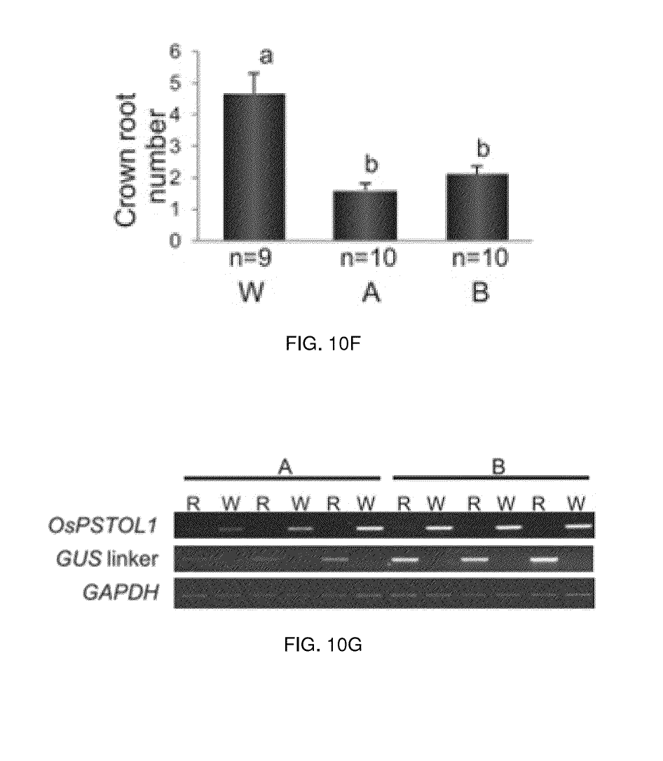

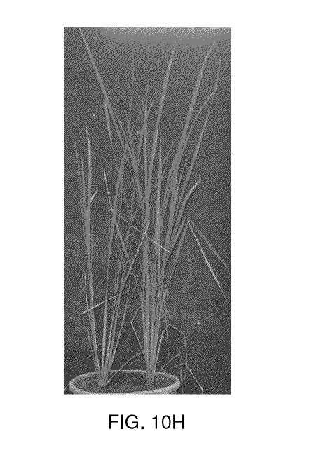

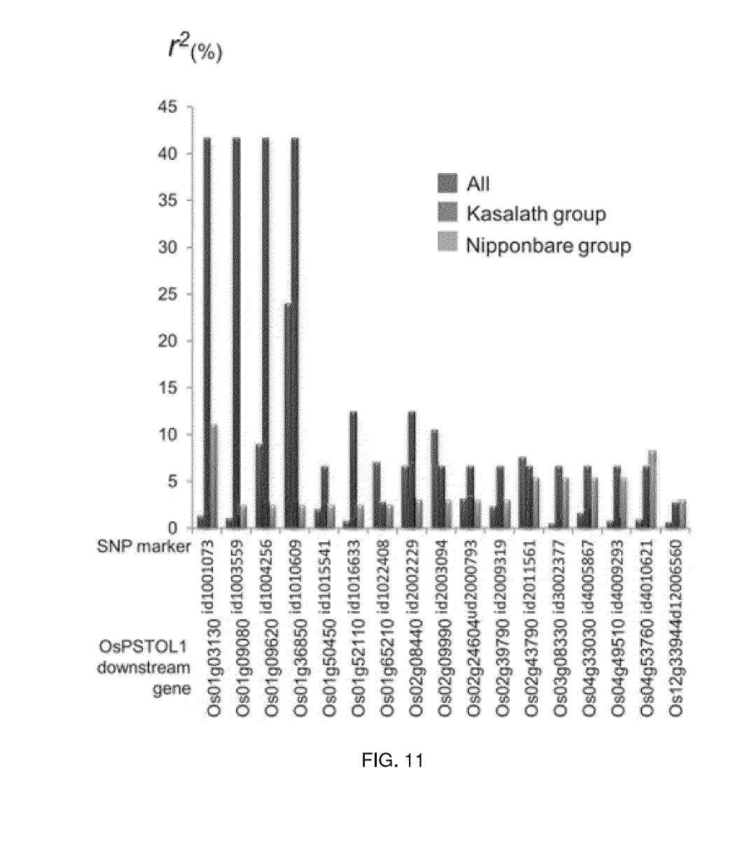

In two different locations with P-deficient soil types, high expression of the OsPSTOL1 transgene (OX high) enhanced grain yield significantly under −P conditions in both indica and japonica (FIGS. 2A-2C; FIG. 7). Transgenic lines with low transgene expression (OX low) were comparable to segregants without the transgene (Null) that were always analyzed in parallel. The data showed that expression of OsPSTOL1 above a certain threshold is required to confer tolerance of P-deficiency. In both varieties, a significantly higher total P content was observed in OX-high lines (FIGS. 2B and 2C). For the IR64 plants, it was further confirmed that the superior performance of OX-high lines was due to a higher root dry weight (FIGS. 2A and 2B). The larger root system also enhanced the uptake of other nutrients, since nitrogen (N) and potassium (K) content were higher in the OX-high lines (FIG. 8). Subsequent phenotypic analyses of IR64-OX lines conducted in nutrient solution with high (100 μM) and reduced (10 μM) P concentrations showed that under both P treatments, total root length and root surface area were significantly higher in transgenic seedlings (FIGS. 3A and 3C). The same experiment was repeated with two different contrasting Pup1 NILs (IR64 and IR74, +/−Pup1) that were developed by marker-assisted introgression of the Kasalath Pup1 locus. In agreement with the above data, seedlings of +Pup1 NILs showed significantly enhanced root growth under high- and low-P conditions (FIGS. 3B, 3C, and 9). The finding that root growth was enhanced in OsPSTOL1 overexpression lines as well as in Pup1 introgression lines demonstrated that OsPSTOL1 is the major tolerance gene within the Pup1 QTL and that this gene acts at least partially independent of P. Down-regulation of OsPSTOL1 by RNAi in Kasalath caused a significant reduction in root number and root surface area, which negatively affected overall plant growth (FIG. 10).

The expression of OsPSTOL1 during root development was then analyzed in more detail by expressing the β-glucuronidase (GUS) reporter gene under the control of the native OsPSTOL1 promoter in transgenic IR64 plants. Specific GUS staining was detected in stem nodes, where, in rice, crown roots were formed that constitute the main root system (FIGS. 3D and 3E). Within the nodes, GUS staining was restricted to crown root primordia and parenchymatic cells located outside of the peripheral vascular cylinder. In older plants, GUS staining was additionally detected in the cells surrounding the nodal vascular anastomoses, which interconnect vascular bundles (FIG. 3E). GUS staining was not evident in older, emerging crown roots or in the initial (seminal) seedling root. The data showed that OsPSTOL1 is a regulator of early crown root development and root growth in rice.

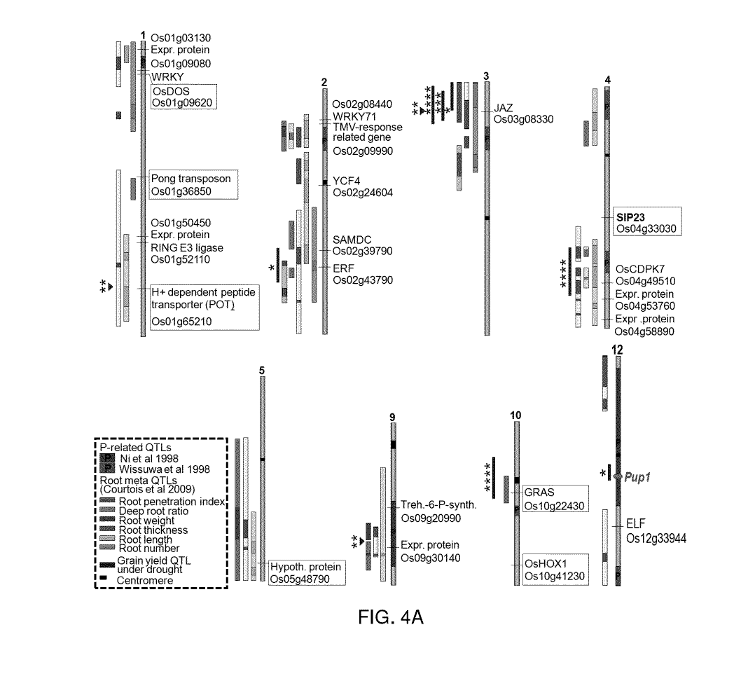

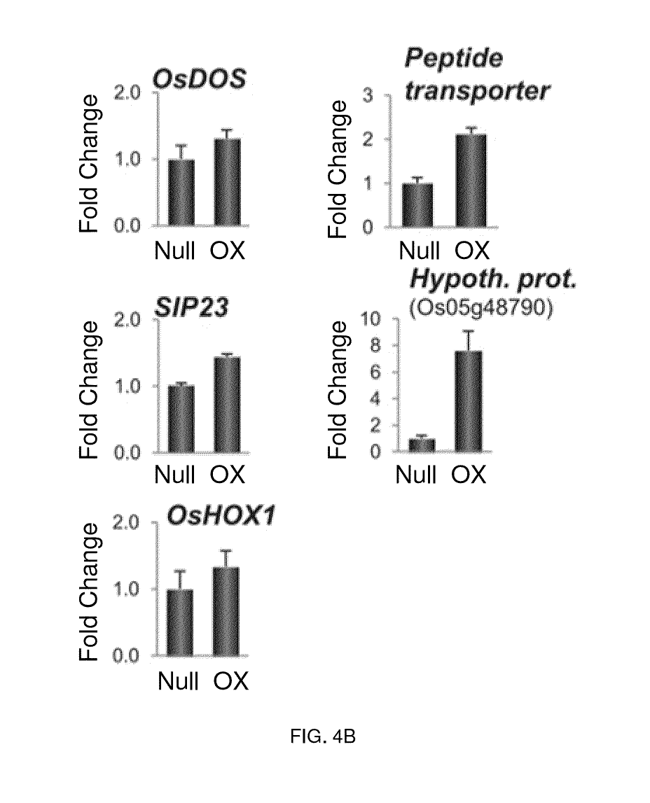

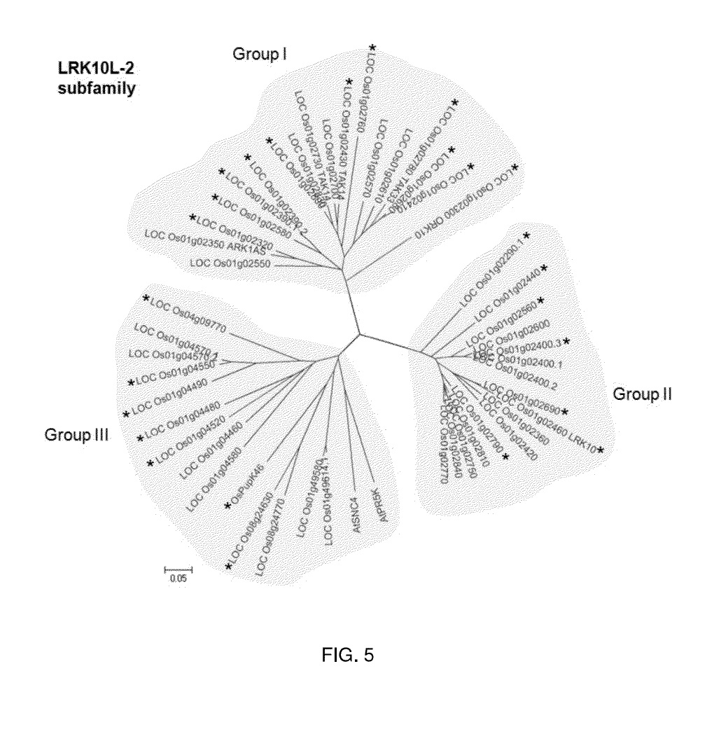

Because OsPSTOL1 is a protein kinase, it cannot directly regulate expression of genes. An Affymetrix gene-array analysis was conducted using root samples from soil-grown IR64 transgenic plants (high OsPSTOL1 overexpression, or OX high) and Null control plants to determine the downstream responses of OsPSTOL1. The data showed that known P-starvation genes were not differentially regulated in the transgenics. Instead, twenty-three genes with constitutively (i.e., independent of the P supply and developmental stage) higher or lower expression were identified in the transgenic plants that are related to root growth and stress response (Table 1). Twenty-one of these differentially expressed genes co-localize with QTLs related to drought tolerance and root growth (FIG. 4A), demonstrating an important role of Pup1/OsPSTOL1 during root development and stress tolerance. In this context, it was also determined that the Pup1 dirigent gene (OsPupk20-2) is downstream of OsPSTOL1 since this gene was specifically induced in 35S::OsPSTOL1 plants and in +Pup1 NILs (FIG. 1F).

To assess whether the expression of the identified genes is independent of P and/or soil-related factors, a qRT-PCR analysis of selected genes was conducted using root RNA samples of 35S::OsPSTOL1 plants grown under high-P conditions in hydroponics. Whereas the data were inconsistent for many of the down-regulated genes, six out of the seven genes with higher expression were specifically induced in 35S::OsPSTOL1 roots (FIG. 4B). Among the genes with higher expression are two transcription factor genes, namely, OsHOX1, a positive regulator of root cell differentiation, and OsDOS, which is known to delay leaf senescence in rice. An association study further showed that a region on chromosome 1, where OsDOS and a WRKY-type transcription factor gene are located, was significantly associated with the presence of OsPSTOL1 in a wider range of tolerant rice accessions.

Marker Assisted Selection and Breeding of P-Deficiency Tolerant Plants

The present disclosure provides molecular markers, (i.e. including marker loci and nucleic acids corresponding to (or derived from) these marker loci, such as probes and amplification products) useful for genotyping plants correlated with the OsPSTOL1 and OsPupK20-2 genes in rice. Such molecular markers are useful for selecting plants that carry the P-deficiency tolerance gene or that do not carry the P-deficiency tolerance gene. Accordingly, these markers are useful for marker assisted selection (MAS) and breeding (marker assisted back crossing—MABC) of P-deficiency tolerant lines and identification of non-tolerant lines. Markers which may be used include markers K46-1, K46-K1, K46-CG1, K46-K2, K46-CG2, and K46-3.

TABLE 1
Genes with Constitutively altered Expression in 35S::OsPSTOL1 Plants

| | | | Gene | | | | | |
|---|---|---|---|---|---|---|---|---|
| | | | expression | | | | | |
| | | | in 35S:: | Affymetrix array data (average of two replicates)* | | | | |
| | | | OsPSTOL1 | 35S:: | | 35S:: | | Gene- |
| | Affymetrix Probe | Anno- | versus | OsPSTOL1 | Null | OsPSTOL1 | Null | related |
| TIGR ID | Set ID | tation | Null controls | (−P) | (−P) | (+P) | (+P) | refs. |
| LOC_Os01g09620 | Os.49042.1.Al_s_at | OsDOS, Zn- | + | 1672.4 | 951.4 | 533.1 | 432.0 | Kong et al |
| | | finger | | | | | | (2006) |
| | | transcription | | | | | | |
| | | factor | | | | | | |
| LOC_Os01g36850 | Os.31233.1.S1_at | Pong type | + | 144.9 | 86.7 | 201.6 | 7.4 | — |
| | | transposon | | | | | | |
| LOC_Os01g65210.1 | Os.7141.1.S1_at | H+ | + | 48.9 | 25.3 | 56.0 | 33.9 | Pao et al |
| | | dependent | | | | | | (1998) |
| | | oligopeptide | | | | | | |
| | | transporter, | | | | | | |
| | | putative | | | | | | |
| LOC_Os04g33030.1 | Os.12183.1.S1_at | SKIP | + | 327.3 | 242.1 | 482.9 | 375.1 | Hou et al |
| | | interacting | | | | | | (2009) |
| | | protein | | | | | | |
| | | SIP23 | | | | | | |
| LOC_Os05g48790.3 | Os.17546.1.S2_a_at | Hypothetical | + | 53.2 | 3.6 | 48.5 | 3.9 | — |
| | | protein | | | | | | |
| LOC_Os10g22430.1 | Os.22472.2.S1_at | GRAS type | + | 684.1 | 542.1 | 534.7 | 354.0 | Day et al |
| | | transcription | | | | | | (2004) |
| | | factor, | | | | | | |
| | | similar to | | | | | | |
| | | CIGR2 | | | | | | |
| LOC_Os10g41230.1 | Os.4605.1.S1_at | OsHox1 | + | 1107.4 | 690.0 | 251.0 | 177.5 | Scarpella |
| | | | | | | | | et al |
| | | | | | | | | (2002) |
| LOC_Os01g03130 | Os.55272.1.S1_at | Expressed | − | 28.4 | 1991.5 | 312.8 | 692.0 | — |
| | | protein | | | | | | |
| LOC_Os01g09080 | Os.3783.1.S1_x_at | WRKY | − | 0.6 | 63.2 | 14.1 | 43.1 | — |
| | | transcription | | | | | | |
| | | factor | | | | | | |
| LOC_Os01g50450 | Os.11717.1.S1_a_at | Expressed | − | 137.6 | 286.1 | 216.6 | 342.4 | — |
| | | protein | | | | | | |
| LOC_Os01g52110 | Os.11450.1.S1_at | RING E3 | − | 296.3 | 1026.1 | 98.8 | 235.1 | Long et al |
| | | ligase | | | | | | (2010); |
| | | | | | | | | Santner |
| | | | | | | | | and |
| | | | | | | | | Estelle |
| | | | | | | | | (2010) |
| LOC_Os02g08440 | OsAffx.24166.1.S1_at | WRKY71 | − | 1.1 | 559.9 | 79.9 | 270.4 | Zou et al |
| | | | | | | | | (2008) |
| LOC_Os02g09990 | Os.55519.1.S1_at | Tobacco | − | 60.0 | 131.5 | 51.6 | 81.7 | — |
| | | mosaic virus | | | | | | |
| | | related | | | | | | |
| | | response | | | | | | |
| | | element, | | | | | | |
| | | putative | | | | | | |
| LOC_Os02g24604 | Os.26509.1.S1_s_at | YCF4 | − | 55.2 | 202.4 | 105.7 | 229.3 | — |
| LOC_Os02g39790 | Os.7972.1.S1_at | S-adenosyl | − | 342.2 | 1675.3 | 633.6 | 810.5 | Takahashi |
| | | methionine | | | | | | and |
| | | decarbox- | | | | | | Kakehi |
| | | ylase (SAMDC) | | | | | | (2010) |
| LOC_Os02g43790 | Os.53660.1.S1_at | ERF type | − | 1141.9 | 1706.2 | 1257.4 | 1750.6 | — |
| | | transcription | | | | | | |
| | | factor | | | | | | |
| LOC_Os03g08330 | Os.9923.1.S1_at | JAZ protein | − | 261.6 | 2324.5 | 627.7 | 1422.1 | Ye et al |
| | | (TIFY/ZIM | | | | | | (2009) |
| | | domain | | | | | | |
| | | protein) | | | | | | |
| LOC_Os04g49510 | Os.282.2.S1_a_at | OsCDPK7 | − | 31.9 | 227.4 | 154.5 | 270.5 | Saijo et al |
| | | | | | | | | (2001) |
| LOC_Os04g53760 | Os.46208.1.S1_x_at | Expressed | − | 8.7 | 91.1 | 57.3 | 173.6 | — |
| | (note: this ID is | protein | | | | | | |
| | miss-annotated as | | | | | | | |
| | LOC_Os10g38292) | | | | | | | |
| LOC_Os04g58890 | Os.48131.1.S1_s_at | Expressed | − | 11.0 | 1307.6 | 160.4 | 639.3 | — |
| | | protein | | | | | | |
| LOC_Os09g20990 | Os.52425.1.S1_x_at | Trehalose-6- | − | 37.1 | 122.7 | 155.7 | 272.3 | — |
| | | P-synthase | | | | | | |
| LOC_Os09g30140 | Os.55282.1.S1_at; | Expressed | − | 186.0 | 329.5 | 124.3 | 256.0 | — |
| | Os.55282.1.S1_x_at | protein | | | | | | |
| LOC_Os12g33944 | OsAffx.1891.1.S1_x_at | ELF domain | − | 43.2 | 91.7 | 40.7 | 102.3 | — |
| | | protein | | | | | | |

+ = constitutively higher expression in 35S::OsPSTOL1 plants;
− = constitutively lower expression in 35S::OsPSTOL1 plants

PSTOL1 and/or PupK20-2 MAS and MABC are described herein.

There are many kinds of molecular markers. For example, molecular markers can include restriction fragment length polymorphisms (RFLP), random amplified polymorphic DNA (RAPD), amplified fragment length polymorphisms (AFLP), single nucleotide polymorphisms (SNP) or simple sequence repeats (SSR). Simple sequence repeats (SSR) or microsatellites are regions of DNA where one to a few bases are tandemly repeated for few to hundreds of times. For example, a di-nucleotide repeat would resemble CACACACA and a trinucleotide repeat would resemble ATGATGATGATG (SEQ ID NO: 54). Simple sequence repeats are thought to be generated due to slippage mediated errors during DNA replication, repair and recombination. Over time, these repeated sequences vary in length between one cultivar and another. An example of allelic variation in SSRs would be: Allele A being GAGAGAGA (4 repeats of the GA sequence) and allele B being GAGAGAGAGAGA (SEQ ID NO: 55) (6 repeats of the GA sequence). When SSRs occur in a coding region, their survival depends on their impact on structure and function of the encoded protein. Since repeat tracks are prone to DNA-slippage mediated expansions/deletions, their occurrences in coding regions are limited by non-perturbation of the reading frame and tolerance of expanding amino acid stretches in the encoded proteins. Among all possible SSRs, tri-nucleotide repeats or multiples thereof are more common in coding regions.

A single nucleotide polymorphism (SNP) is a DNA sequence variation occurring when a single nucleotide—A, T, C or G—differs between members of a species (or between paired chromosomes in an individual). For example, two sequenced DNA fragments from two individuals, AAGCCTA to AAGCTTA, contain a difference in a single nucleotide. In this case, there are two alleles: C and T.

A primary motivation for development of molecular markers in crop species is the potential for increased efficiency in plant breeding through marker assisted selection (MAS) and marker assisted backcrossing (MABC). Genetic marker alleles, or alternatively, identified QTL alleles, are used to identify plants that contain a desired genotype at one or more loci and that are expected to transfer the desired genotype, along with a desired phenotype to their progeny. Genetic marker alleles can be used to identify plants that contain a desired genotype at one locus or at several unlinked or linked loci (e.g., a haplotype) and that would be expected to transfer the desired genotype, along with a desired phenotype to their progeny. The present disclosure provides the means to identify plants, particularly rice, that are able to improve the P-deficiency tolerance of rice by identifying plants having a specified gene, e.g., PSTOL1 and/or PupK20-2, and homologous or linked markers. Similarly, by identifying plants lacking the desired allele, non-P-deficiency tolerant plants can be identified and, e.g., eliminated from subsequent crosses.

After a desired phenotype, e.g., P-deficiency tolerance and a polymorphic chromosomal locus, e.g., a marker locus or QTL, are determined to segregate together, it is possible to use those polymorphic loci to select for alleles corresponding to the desired phenotype: a process called marker-assisted selection (MAS). In brief, a nucleic acid corresponding to the marker nucleic acid is detected in a biological sample from a plant to be selected. This detection can take the form of hybridization of a probe nucleic acid to a marker, e.g., using allele-specific hybridization, Southern analysis, northern analysis, in situ hybridization, hybridization of primers followed by PCR amplification of a region of the marker, or the like. After the presence (or absence) of a particular marker and/or marker allele in the biological sample is verified, the plant is selected, i.e., used to make progeny plants by selective breeding.

P-deficiency tolerance screening for large numbers of plants can be expensive, time consuming and unreliable. Use of the polymorphic loci described herein, and genetically-linked nucleic acids, as genetic markers for the P-deficiency tolerance locus is an effective method for selecting varieties capable of fertility restoration in breeding programs. One advantage of marker-assisted selection over field evaluations for P-deficiency tolerance is that MAS can be done at any time of year regardless of the growing season. Moreover, environmental effects are irrelevant to marker-assisted selection.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent into an otherwise desirable genetic background from the recurrent parent. The more cycles of backcrossing that are done, the greater the genetic contribution of the recurrent parent to the resulting variety. This is often necessary, because donor parent plants may be otherwise undesirable, i.e., due to low yield, low fecundity, or the like. In contrast, varieties which are the result of intensive breeding programs may have excellent yield, fecundity or the like, merely being deficient in one desired trait such as P-deficiency tolerance. Backcrossing can be done to select for or against a trait.

Markers corresponding to genetic polymorphisms between members of a population can be detected by numerous methods (e.g., restriction fragment length polymorphisms, isozyme markers, allele specific hybridization (ASH), amplified variable sequences of the plant genome, self-sustained sequence replication, simple sequence repeat (SSR), single nucleotide polymorphism (SNP) or amplified fragment length polymorphisms (AFLP)).

The majority of genetic markers rely on one or more properties of nucleic acids for their detection. For example, some techniques for detecting genetic markers utilize hybridization of a probe nucleic acid to nucleic acids corresponding to the genetic marker. Hybridization formats include but are not limited to, solution phase, solid phase, mixed phase or in situ hybridization assays. Markers which are restriction fragment length polymorphisms (RFLP), are detected by hybridizing a probe (which is typically a sub-fragment or a synthetic oligonucleotide corresponding to a sub-fragment of the nucleic acid to be detected) to restriction digested genomic DNA. The restriction enzyme is selected to provide restriction fragments of at least two alternative (or polymorphic) lengths in different individuals and will often vary from line to line. Determining a (one or more) restriction enzyme that produces informative fragments for each cross is a simple procedure, well known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing. Nucleic acid probes to the marker loci can be cloned and/or synthesized. Detectable labels suitable for use with nucleic acid probes include any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling markers is readily achieved such as by the use of labeled PCR primers to marker loci.

The hybridized probe is then detected using, most typically, autoradiography or other similar detection techniques (e.g., fluorography, liquid scintillation counter, etc.). Examples of specific hybridization protocols are widely available in the art.

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are well known in the art. Examples of techniques sufficient to direct persons of skill through such in vitro methods, including the polymerase chain reaction (PCR), the ligase chain reaction (LCR), O,β-replicase amplification and other RNA polymerase mediated techniques (e.g., NASBA), are found in Mullis, et al., (1987) U.S. Pat. No. 4,683,202; PCR Protocols, A Guide to Methods and Applications (Innis, et al., eds.) Academic Press Inc., San Diego Academic Press Inc. San Diego, Calif. (1990) (Innis); Arnheim and Levinson, (Oct. 1 , 1990) C&EN 36-47; The Journal Of NIH Research (1991) 3:81-94; Kwoh, et al., (1989) Proc. Natl. Acad. Sci. USA 86:1173; Guatelli, et al., (1990) Proc. Natl Acad. Sci. USA 87:1874; Lomell, et al., (1989) J. Clin. Chem 35:1826; Landegren, et al., (1988) Science 241 :1077-1080; Van Brunt, (1990) Biotechnology 8:291-294; Wu and Wallace, (1989) Gene 4:560; Barringer, et al., (1990) Gene 89:117; and Sooknanan and Malek, (1995) Biotechnology 13:563-564. Improved methods of cloning in vitro amplified nucleic acids are described in Wallace, et al., U.S. Pat. No. 5,426,039. Improved methods of amplifying large nucleic acids by PCR are summarized in Cheng, et al., (1994) Nature 369:684, and the references therein, in which PCR amplicons of up to 40kb are generated. One of skill will appreciate that essentially any RNA can be converted into a double stranded DNA suitable for restriction digestion, PCR expansion and sequencing using reverse transcriptase and a polymerase.

Oligonucleotides for use as primers, e.g., in amplification reactions and for use as nucleic acid sequence probes, are typically synthesized chemically according to the solid phase phosphoramidite triester method described by Beaucage and Caruthers, (1981) Tetrahedron Lett. 22:1859 or can simply be ordered commercially.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) Rnase H and (3) a DNA-dependent RNA polymerase (Guatelli, et al., (1990) Proc Natl Acad Sci USA 87:1874). By mimicking the retroviral strategy of RNA replication by means of cDNA intermediates, this reaction accumulates cDNA and RNA copies of the original target.

As mentioned above, there are many different types of molecular markers, including amplified fragment length polymorphisms (AFLP), allele-specific hybridization (ASH), single nucleotide polymorphisms (SNP), simple sequence repeats (SSR) and isozyme markers. Methods of using the different types of molecular markers are known to those skilled in the art.

SSR data is generated by hybridizing primers to conserved regions of the plant genome which flank the SSR sequence. PCR is then used to amplify the repeats between the primers. The amplified sequences are then electrophoresed to determine the size and therefore the di-, tri and tetra nucleotide repeats.

The presence of PSTOL1 and/or PupK20-2 gene or a homolog thereof, in the genome of a plant exhibiting a preferred phenotypic trait is determined by any method listed above, e.g., RFLP, AFLP, SSR, etc. If the nucleic acids from the plant are positive for a desired genetic marker, the plant can be selfed to create a true breeding line with the same genotype or it can be crossed with a plant with the same marker or with other desired characteristics to create a sexually crossed hybrid generation.

It will be recognized by one skilled in the art that the materials and methods of the present disclosure may be similarly used to confer tolerance of P-deficiency in cereal grasses other than rice, such as corn, wheat, barley, sorghum, millet, oats, and rye

### EXAMPLES

### Example 1

### Conference of Tolerance of Phosphorus Deficiency—OsPSTOL1

Quantitative RT-PCR of Pup1 candidate genes

Seeds of near isogenic lines (NILs) segregating for the Pup1 locus (+Pup1: NILs 6-4, Y-4, 14-4; Pup1: NILs Y6, Y10, and Nipponbare) (J. H. Chin et al., Theor Appl Genet, Vol. 120, pp. 1073-1086, 2010; M. Wissuwa et al., Theor Appl Genet, Vol. 105, pp. 890-897, 2002) were sown directly in pots filled with P-deficient and P-fixing Andosol from a field located at Tsukuba, Japan, that had not received P-fertilizer throughout its 40-year cropping history (−P). An equivalent of 60 kg P ha-1 was applied for the control treatment (+P). Pots were initially watered every 2-3 days and afterwards the soil was kept field capacity. The experiment was conducted in a completely randomized design with three replications and four plants per replicate pot. Root tissue samples were taken at 49 days after sowing. Total RNA was extracted using the RNeasy Mini Kit according to the instructions of the manufacturer (Qiagen) and treated with RNase-free DNase I (Qiagen). Quantitative RT-PCR was performed. cDNA synthesis was conducted at 37° C. for 15 min followed by 5 sec of 85° C. using 500 ng DNase-treated total RNA with PrimeScript RT reagent kit (Takara, Japan). Quantitative RT-PCR was performed with 10 ng RT template and SYBR Premix ExTaq (Perfect Real Time, Takara, Japan). PCR cycle conditions were 94° C. for 10 sec as the first denaturing step, followed by 40 cycles at 94° C. for 5 sec, 55° C. to 60° C. for 10 sec, and 72° C. for 15 sec, and a gradual increase in temperature from 55° C. to 96° C. during the dissociation stage to monitor the specificity of each primer pair. Rice Os18S was used as an internal control. For primer sequences, see Table 2. Expression levels were calculated using the delta-delta comparison and expressed as fold changes under −P relative to expression under +P conditions (expression=1).

In vitro Phosphorylation Assay

Seeds of Arabidopsis thaliana ecotype Col-0 and of the stn7 stn8 double mutant were sown in plastic trays containing one portion of Techinc and one portion of Flox 6 soils and incubated for 3 days at 5° C. in the dark to break the dormancy. Plants were grown in a greenhouse under long-day conditions (16 h light/8 h dark) for 4 weeks. Thylakoids were isolated from 4-week-old plants in the presence of the phosphatase inhibitor NaF (10 mM). The coding sequence (CDS) of OsPSTOL1 was cloned into pBAD-DEST49 vector (Invitrogen), and recombinant OsPSTOL1 (OsPSTOL1_{rec}) was expressed in the E. coli strain BL21 with a C-terminal 6× His-tag (SEQ ID NO: 56). OsPSTOL1_{rec} was purified under denaturing conditions following a Ni-NTA batch purification procedure according to the instructions of the manufacturer (Qiagen). After protein precipitation in 10% trichloroacetic acid (TCA) followed by three washing steps with absolute ethanol, around 500 μg of OsPSTOL1_{rec} protein was resuspended in 500 μl of 1% (w/v) lithium dodecyl sulfate (LDS), 12.5% (w/v) sucrose, 5 mM e-aminocaproic acid, 1 mM benzamidine, and 50 mM HEPES KOH (pH 7.8). Subsequently, OsPSTOL1_{rec} protein was boiled for 2 min at 100° C. and incubated for 15 min at 25 ° C. Then, dithiothreitol (DTT; 75 mM final concentration) was added and the solution was subjected to three freezing-thawing cycles (20 min at −20° C., 20 min at −80° C., 20 min at −20° C., thawing in an ice-water bath, and 5 min at 25° C.). After completion of the three freezing-thawing cycles, octyl-glucopyranoside (OGP; 1% [w/v] final concentration) was added and the solution was kept on ice for 15 min before KC1 (75 mM, final concentration) was added to precipitate the LDS detergent. After centrifugation at 16,000g at 4° C. for 10 min, the supernatant containing the refolded OsPSTOL1_{rec} in the presence of 1% (w/v) OGP was collected. Subsequently, 1 μl of kinase was incubated together with thylakoids corresponding to 5 μg of total chlorophyll. The phosphorylation reaction was performed in 50 μl total volume containing 0.06% (w/v) dodecyl-β-D-maltoside, 5 mM Mg-acetate, 5 mM DTT, 100 mM HEPES KOH (pH 7.8), 200 mM ATP, and 10 mM NaF at 37° C. for 2 h. The reaction mixture was loaded on an SDS-PAGE and immunoblot analyses with phosphothreonine-specific antibodies (Cell Signaling) were performed as described (32). A replicative SDS-PAGE was stained with Coomassie Brilliant Blue.

TABLE 2
Primers Used

| | | Primer Sequence | Amplicon | SEQ ID |
|---|---|---|---|---|
| Expermient | Primer name | (5′-3′) | Size (bp) | NO: |
| OsPupK04 qRT-PCR | OsPupK04-F | TCAAGCTTGTGGTGCACTTG | 168 | 11 |
| | OsPupK04-R | CTCCTCCTGAACTCATTGTACC | | 12 |
| OsPupK05 qRT-PCR | OsPupK05-F | AGTACAGTCCGGCGTCATAC | 161 | 13 |
| | OsPupK05-R | CCGAGATCTGGTCCTCAATA | | 14 |
| OsPupK20 qRT-PCR | OsPupK20-F | GCACAAGGATGGCATATCGT | 166 | 15 |
| | OsPupK20-R | TCCCACCCATAATAGACCACTC | | 16 |
| OsPupK29 qRT-PCR | OsPupK29-F | AGGTCGACAGCCTTAGAATAGC | 163 | 17 |
| | OsPupK29-R | CTGGTGAGAAACATAGAGCCGT | | 18 |
| OsPSTOL1 qRT-PCR | OsPSTOL1-F | GTTTGTGGTGCATACAACTCGT | 165 | 19 |
| | OsPSTOL1-R | GGTTCCTCAAAAACAGAAGATG | | 20 |
| Os18S qRT-PCR | Os18S-F | ATGATAACTCGACGGATCGC | 169 | 21 |
| | os18S-R | CTTGGATGTGGTAGCCGTTT | | 22 |
| Sequencing for 35S- | oRG89 | TTCGCAAGACCCTTCCTCTA | — | 23 |
| OsPSTOL1 fusion | | | | |
| Sequencing for | oSH07 | GAGTACATGCCCAATGGTTC | — | 24 |
| OsPSTOL1-Nos | | | | |
| terminator fusion | | | | |
| OsPSTOL1 RT-PCR | oKas4603 | ATGCTGCTCTGTCAAAGGGCAT | 980 | 25 |
| in 35S::OsPSTOL1 | oKas4604 | CAAGCTCAAAGCCCTTTTGGTG | | 26 |
| plants; sequencing | | | | |
| of OsPSTOL1 CDS | | | | |
| PCR of | oRG88 | CCAGCTCAGGGTGTTATCTC | 560 | 27 |
| 35S::OsPSTOL1 | oRG89 | TTCGCAAGACCCTTCCTCTA | | 28 |
| transgene | | | | |
| PCR of HPT gene | oRG127 | GGTTGGCTTGTATGGAGCAG | 258 | 29 |
| | oRG128 | CTTCTACACAGCCATCGGTC | | 30 |
| PCR/RT-PCR of | GAPDH-F | GCAGGAACCCTGAGGAGATC | 650/365 | 31 |
| GAPDH gene | GAPDH-R | TTCCCCCTCCAGTCCTTGCT | | 32 |
| LOC_Os01g09620 | oRG146 | TCCGGGAGAAGGTGTTCGAG | 318 | 33 |
| qRT-PCR | oRG147 | CCTCCTCTCCACCAACCATG | | 34 |
| LOC_Os01g65210 | oRG209 | AGTGCGGCTTCTCCTAGCTG | 230 | 35 |
| qRT-PCR | oRG210 | GTCAGCAGTGGAGGAGAACG | | 36 |
| LOC_Os04g33030 | oRG211 | TCGCTCCATGTCCTGCTGTC | 165 | 37 |
| qRT-PCR | oRG212 | GCTCAGTGTCCGCCAAGATC | | 38 |
| LOC_Os05g48790 | oRG177 | GCAGTCAGTGACATGTTTGATCAAC | 187 | 39 |
| qRT-PCR | oRG178 | CTTAGCACTCACATCGGAGAC | | 40 |
| LOC_Os10g41230 | oRG215 | CATCGAGATGCCGTTCCTGC | 403 | 41 |
| qRT-PCR | oRG216 | CGTCTGCTTCAGCTTCGTCC | | 42 |
| OsPSTOL1 promoter | oRG107 | CAGTAATTTTGGATATATGGG | 1,755 | 43 |
| cloning | oRG109 | TAATCCGTAACGTTTCTTGTGC | | 44 |
| PCR and sequencing | oRG120 | CGGTTACTAGCGTGGTTTCG | 487 | 45 |
| of OsPSTOL1 | oRG134 | CTTTCCCACCAACGCTGATC | | 46 |
| promoter::GUS | | | | |
| fusion | | | | |
| Cloning of | oSH07 | GAGTACATGCCCAATGGTTC | 322 | 47 |
| OsPSTOL1 gene | oSH08 | ACTGCCTGGAAAACACTTCA | | 48 |
| fragment for RNAi | | | | |
| cassette | | | | |
| RT-PCR of actin | actin-F | TTGCTGACAGGATGAGCAAG | | 49 |

Generation of 35S::OsPSTOL1 Transgenic Plants

The CDS of OsPSTOL1 was amplified from Kasalath genomic DNA using the primer pair oKas4603/oKas4604 (all primer sequences are provided in Table 2), cloned into pCR8/GW/TOPO TA cloning vector (Invitrogen) and sent for sequencing (Macrogen, Korea). Through LR clonase recombination reaction (Invitrogen), the CDS was sub-cloned into the pMDC32 binary destination vector (M. D. Curtis & U. A. Grossniklaus, Plant Physiol, Vol. 133, pp. 462-469, 2003) containing the 35S promoter and NOS-terminator (35S::OsPSTOL1). The construct was sequenced using primer pairs amplifying the 35S promoter (oRG89) and the NOS-terminator (oSH07) with adjacent CDS, respectively. The correct sequence of OsPSTOL1 was re-confirmed by sequencing with the primer pair oKas4603/oKas4604. Transformation of the construct into the indica-type IR64 and japonica-type Nipponbare rice varieties, which naturally lack the OsPSTOL1 gene, was mediated by Agrobacterium tumefaciens strain LBA4404. Transgenic plants were tested by genomic PCR in the T1 generation for the presence of the hygromycin phosphotransferase gene (HPT; primer pair oRG127/oRG128) and the 35S promoter with part of the CDS (primer pair oRG89/oRG88). PCR was carried out in a total volume of 20 μl with the following conditions: 100 ng genomic DNA, primers (0.2 μM each of forward and reverse), 1×PCR buffer, 0.5 mM dNTP mix, and 1.5 U Taq DNA polymerase (i-Taq DNA Polymerase, INTRON Biotechnology Inc.). The PCR cycle settings were 94° C. for 5 min, followed by 30 cycles of 94° C. for 30 sec, Ta (55° C. for primers oRG127 and oRG128; 60° C. for primers oRG88 & oRG89 and GAPDH-F & GAPDH-R) for 30 sec, 72° C. for extension time (30 sec for primers oRG127/oRG128; 45 sec for primers oRG88/oRG89 and GAPDH-F/GAPDH-R), and a final extension at 72° C. for 10 min. As a control, the cytosolic glyceraldehyde-3-phosphate dehydrogenase (GAPDH) gene was amplified using the primer pair GAPDH-F/GAPDH-R. PCR products were separated by agarose gel electrophoresis and stained with SYBR Safe (Invitrogen). The copy number of the transgene in selected plants was determined by Southern blot analysis using genomic DNA digested with XbaI and Sad, respectively, and hybridized with a DIG-labeled HPT probe. Plants with independent transformation events were selected for phenotypic analysis in the T1 generation (FIG. 6).

Phenotyping of 35S::OsPSTOL1 Plants

T1 seeds from selected independent IR64 transgenic lines (FIG. 6B) were pre-germinated in Petri dishes for 3 d in the dark at room temperature before seedlings were transferred into seedling trays. At 21 days after germination (DAG), transgenic plants and the corresponding Null segregant were transferred into pots filled with P-deficient soil (P-Bray: 1.23±0.30 mg kg⁻¹; P-Olsen: 0.77±0.46 mg kg⁻¹ ) from Siniloan (Luzon, Philippines). To control for pot-to-pot variation, one transgenic plant and one Null segregant were always grown together in each pot. Each pot received the equivalent of 90 kg N ha⁻¹, 40 kg K ha⁻¹, and 20 kg Zn ha⁻¹. The equivalent of 60 kg P ha⁻¹ was applied only to the +P control treatment that was done in parallel. To mimic upland field conditions, plants grown under −P conditions were exposed to a dry-down treatment until leaf rolling at about 60 days after germination. Control pots were kept well watered but aerobic.

In an initial experiment, seven independent lines and the corresponding Nulls were analyzed and two lines (#19, #20) with high transgene expression and three lines (#5, #14, #21) with low transgene expression (FIGS. 8 and 9) were selected for detailed analyses. A similar phenotyping experiment was conducted at JIRCAS using independent T2 Nipponbare transgenic lines grown in well-watered (aerobic) P-deficient soil from Tsukuba (Japan) (FIG. 6A). For the +P control, soil from a field that had regularly received P-fertilizer was used and 60 kg P ha⁻¹ was additionally applied.

Macronutrients in roots, shoots, and grains of IR64 transgenic plants and Null controls were analyzed by the Analytical Services Laboratory at IRRI. The Kjeldahl method was used to determine the % N while a modified ASL nitric/perchloric acid digestion was done for ICP analysis of P and K.

Semi-Quantitative RT-PCR analysis of Transgene Expression

RT-PCR analysis of 35S::OsPSTOL1 expression was conducted using leaf samples. Total RNA was extracted using Trizol (Invitrogen) or RNeasy Mini Kit (Qiagen) and DNA contaminations were removed with RNase-free DNase I (Promega or Qiagen). cDNA synthesis in the IR64 experiment was performed at 55° C. for 1 h in a 20 μl reaction with 1 μg RNA template, 2.5 μM oligo dT, 0.5 mM dNTP mix, 0.01 M DTT, 1× first-strand buffer, and 200 U of Superscript III RT (Invitrogen). For the Nipponbare experiment, 500 ng RNA template was used for cDNA synthesis in a total volume of 10 μl using PrimeScript RT reagent kit (Takara, Japan) at 37° C. for 15 min followed by 85° C. for 5 sec. For standard PCR analyses, 0.5-1 μl cDNA was used as template for amplification of the transgene with i-Taq DNA polymerase (INTRON Biotechnology, Inc.) or Takara Taq (Takara, Japan) using gene-specific primers (0.2 μM each, oKas4603 and oKas4604; Table 2). GAPDH was used as a positive control.

Root Scan of IR64 35S::OsPSTOL1 T2 Plants and Pup1 NILs Grown in Hydroponics

Seeds of the IR64 T2 transgenic line #20 and seeds of IR64-Pup1 and IR74-Pup1 NILs were pre-germinated in Petri dishes in the dark at room temperature. After three days, germinated seeds were transferred to Yoshida culture solution with 100 μM and 10 μM NaH₂PO₄, respectively. The solution was replaced every three days. Total root length and root surface area of seedlings (11-21 DAG) were measured using WinRhizo (MAC STD1600, Regent Instruments). Each root system was evenly spread out and scanned at least twice to obtain average values. Each experiment was reproduced at least once. Null controls and NILs without Pup1 were always grown and analyzed in parallel.

OsPSTOL1 Promoter::GUS Transgenic Plants (IR64 Variety)

The 1,755-bp promoter of OsPSTOL1 was amplified from the genomic DNA of +Pup1 NILs using the primer pair oRG107/oRG109 (Table 2), cloned into pCR8/GW/TOPO TA cloning vector (Invitrogen) and sent for sequencing (Macrogen, Korea). The promoter fragment was sub-cloned into pMDC164 binary destination vector (A. Haldrup et al., Plant, Vol. 17, pp. 689-698, 1999) through LR clonase recombination reaction (Invitrogen). The final construct contained the GUS gene driven by the OsPSTOL1 promoter, which was confirmed using the forward primer oRG120 sequencing from the 3′-end of the promoter extending to the CDS of the GUS gene. The construct was transformed into IR64 using the same protocol as described above. Transformed T0 plants were identified by genomic PCR using oRG120/oRG134 verifying also the fusion of the OsPSTOL1 promoter with the GUS gene. PCR conditions were the same as described above. For expression analyses, one-week-old T1 seedlings grown in Petri dishes at room temperature in the dark were incubated in GUS staining solution. Samples were stored in 70% ethanol before embedding in agarose for sectioning (200 μm) and bright field microscopy (Olympus BX53 with attached Olympus DP70 camera).

Affymetrix Gene-Expression Analysis

For microarray analyses, root samples of IR64 35S::OsPSTOL1 and the corresponding Null segregants were collected from T1 plants of line #20 grown in pots with P-deficient soil under stress (−P, dry-down) and control (+P-fertilizer, well-watered aerobic) conditions. Plants grown under control conditions were sampled at the four-tiller stage at 33 DAG. The stress treatment delayed development, and plants were harvested at the heading stage when plants had developed two to four tillers. For all treatments, samples of two biological replicates were analyzed. Total root RNA was extracted using Trizol according to the instructions from the manufacturer (Invitrogen) with modifications. The RNA was re-precipitated by adding 2.5× volume absolute ethanol and one-tenth volume 3M NaOAc (pH 5.2), washed twice (70% and 100% ethanol), air-dried, and dissolved in RNase-free water before treatment with RNase-free DNase I (Promega). cRNA synthesis and labeling, hybridization, and data analysis with the GeneChip Operating System 1.4 were performed by ATLAS Biolabs GmbH (Germany) using Affymetrix GeneChip Rice Genome Arrays. Identification of genes with differential expression between transgenics and Nulls and between P treatments was restricted to probe-set IDs with consistent data in both replicates. For the identification of genes with lower expression in transgenic plants compared with Nulls, all IDs “present” (expressed) in the Nulls were used. For the identification of genes with higher expression, all IDs present in transgenic plants were used. Genes classified as “constitutively” changed in the transgenics showed significantly (p<0.05) altered expression in all data sets.

Expression Analysis and Physical Location of Putative OsPSTOL1 Downstream Genes

For qRT-PCR analysis of the genes indentified in the Affymetrix study, roots from IR64 35S::OsPSTOL1 T2 and Null control plants grown hydroponically in Yoshida culture solution with 100 μM P were collected at 49 DAG. Total RNA extracted with Trizol (Invitrogen) was treated with RNase-free DNase I (Promega) and cDNA synthesis was performed with Transcriptor First Strand cDNA Synthesis Kit (Roche) using 1μg DNase-treated RNA. qRT-PCR was conducted with LightCycler 480 SYBR Green I Master (Roche) using 0.5 μl cDNA template with the following PCR conditions: 94° C. for 5 min, 40 cycles at 94° C. for 10 sec, 55° C. for 5 sec, and 72° C. for 20 sec. Primer sequences are provided in Table 2. GAPDH was used as an internal control. Expression levels were calculated using the delta-delta comparison and expressed as fold change relative to the expression in Null controls (expression=1). The physical location of genes was derived from the Rice Genome browser (http://rice.plantbiology.msu.edu/cgi-bin/gbrowse/rice/) and the physical position of drought-tolerance and meta-QTLs for roots and drought was derived from published data (M. Wissuwa et al., Theor Appl Genet, Vol. 97, pp. 777-783; J. Bernier et al., Crop Sci, Vol. 47, pp. 507-518, 2007; I. K. Bimpong et al., J Plant Breed Crop Sci, Vol. 3, pp. 60-67, 2011; B. Courtois et al., Rice, Vol. 22, pp.115-128, 2009; M. S. Gomez et al., Am J Biochem Biotechnol, Vol. 2, pp. 161-169, 2006; J. C. Lanceras et al., Plant Physiol, Vol. 135, pp. 1-16, 2004; J. J. Ni et al. Theor Appl Genet, Vol. 97, pp. 1361-1369, 1998). The data were manually summarized and graphically illustrated.

Association Analysis

Seventy-nine rice varieties with different Pup1 haplotypes (J. H. Chin et al., Plant Physiol, Vol. 156, pp. 1202-1216, 2011) were genotyped with 379 SNP markers using the RiceOPA2.1 BeadXpress platform (M. J. Thomson et al., Mol Breeding, 2011) and analyzed using STRUCTURE (J. K. Pritchard et al., Genetics, Vol. 155, pp. 945-959, 2000) to identify co-ancestry subgroups. The optimum number of populations (K) was selected by testing for K=1 to K=8 using ten independent runs of 10,000 burn-in runs followed by 100,000 iterations with a model allowing for admixture and correlated allele frequencies (D. Falush et al., Genetics, Vol. 164, pp. 1567-1587, 2003). K=6 provided the best distinction and two subgroups with the most contrasting Pup1 haplotypes (Kasalath type: +Pup1 ; Nipponbare type: −Pup1) were selected for further analysis. SNP markers located within the putative OsPSTOL1 downstream genes (FIG. 4) are not present in the 379 SNP set and markers located within approximately 1 Mb distance from the genes were therefore used for analysis of allelic associations with OsPSTOL1 using TASSEL 3.0 (P. J. Bradbury et al., Bioinformatics, Vol. 23, pp. 2633-2635, 2007) (FIG. 11). Rice accessions included in this study: Kasalath, AUS196, AUS257, Dular, IR84144-11-12, Lemont, Vandana; Bala, CT6510-24-1-2, IR 42, IR64, IR66424-1-2-1-5, IR73678-6-9-B, IR 74, IR74371-46-1-1, K36-5-1-1BB, Nipponbare, PM-36, Vary Lava 701.

RNA Interference (RNAi) Transgenic Plants

A 322-bp fragment specific to the OsPSTOL1 gene was amplified using the primer pair oSH07/oSH08 and cloned into pENTR/D-TOPO vector (Invitrogen). The cloned fragment was transferred into pANDA RNAi vector (D. Miki & K. Shimamoto, Plant Cell Physiol, Vol. 45, pp. 490-495, 2004) through LR clonase recombination reaction (Invitrogen). The RNAi construct was transformed into the Pup1 donor variety Kasalath using the rice transformation protocol described above. Six RNAi lines (T2 and T3 generation) were selected based on semi-quantitative RT-PCR showing down-regulation of OsPSTOL1 in roots using the oSH07/oSHO8 primer pair as described above. To verify whether the RNAi cassette is active, the expression of the GUS linker between the sense and antisense sequence (D. Miki & K Shimamoto, Plant Cell Physiol, Vol. 45, pp. 490-495, 2004) of the cloned OsPSTOL1 fragment was determined. Selected RNAi lines were grown in hydroponics culture solution and in P-deficient soil and phenotyped (FIG. 10). Wild-type Kasalath and Null segregants were analyzed in parallel.

### Example 2

### Conference of Tolerance of Phosphorus Deficiency—OsPupK20-2

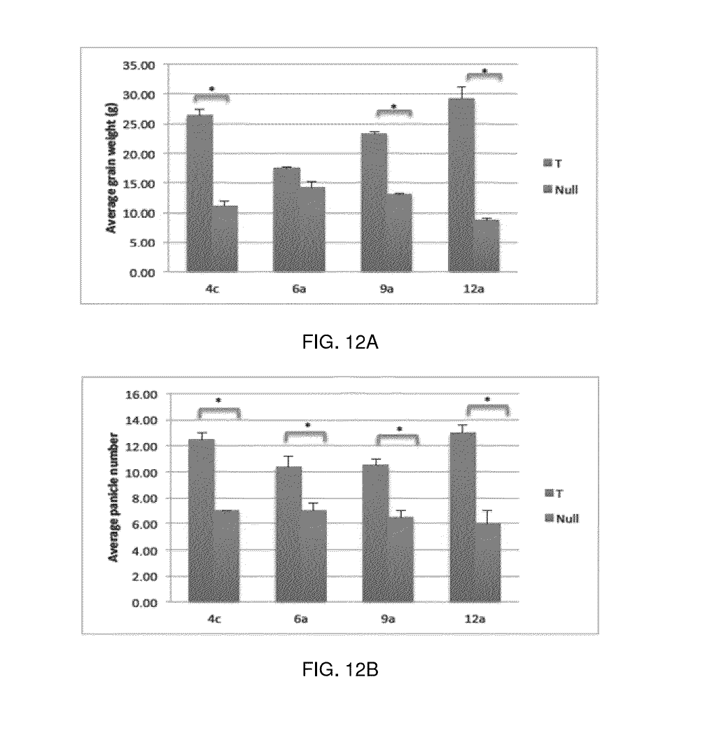

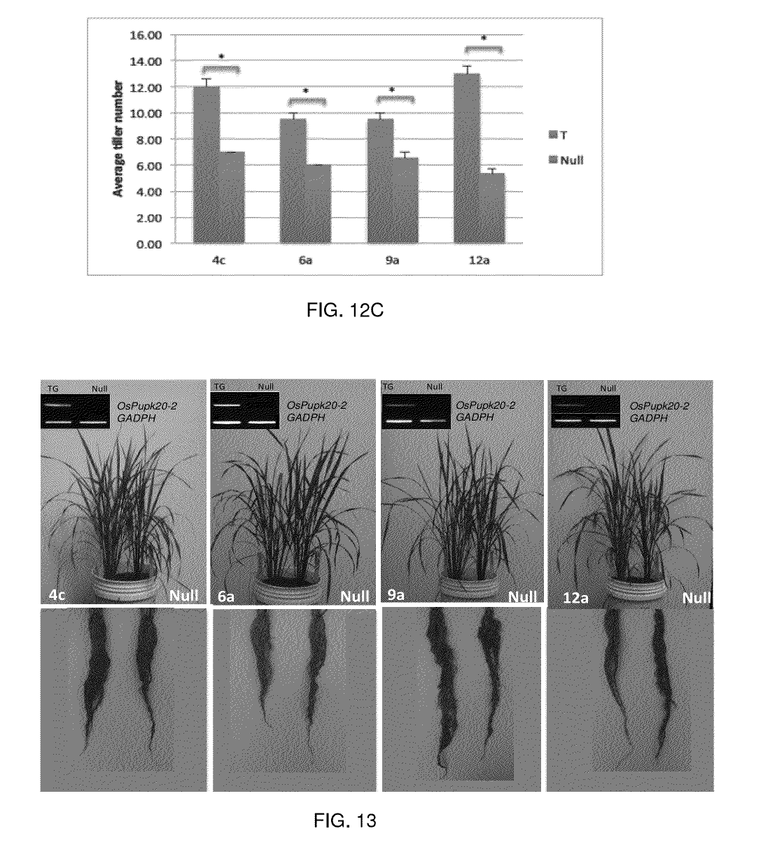

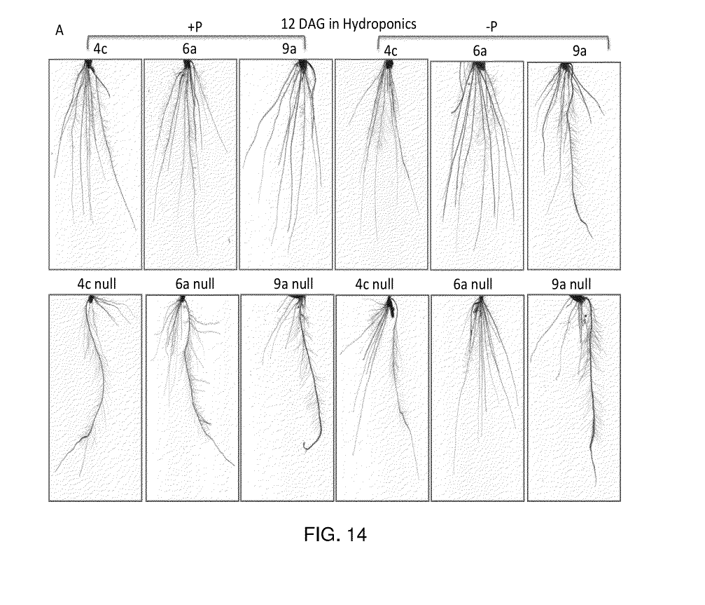

Phenotyping of OsPupK20-2 (dirigent) overexpressing lines were done for three generations. The transgenics had better grain yield compared to corresponding nulls (FIG. 12). Transgenics in all lines also showed greater panicle number and higher average tiller number (FIG. 12). Transgenic plants overexpressing OsPupK20-2 also showed enhanced root growth (FIGS. 13-15).

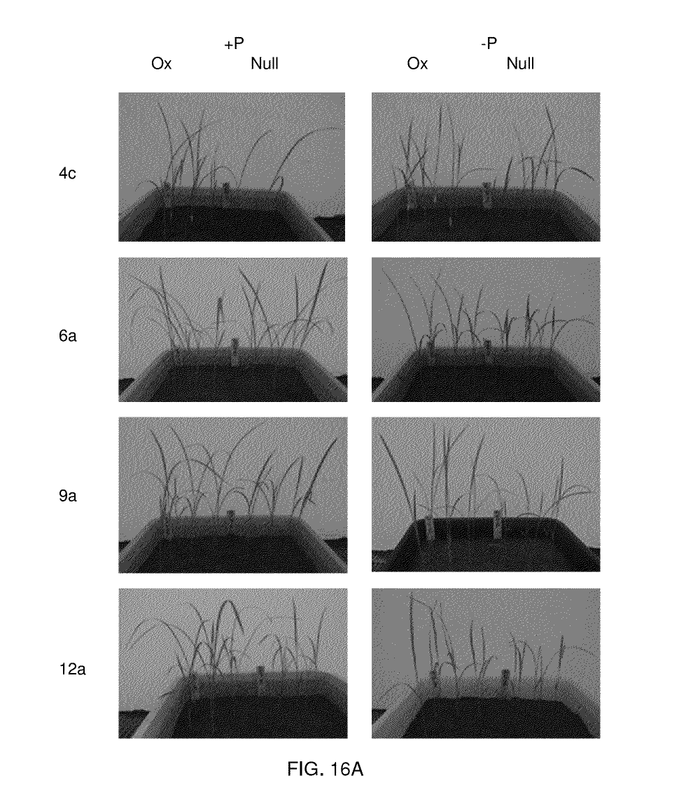

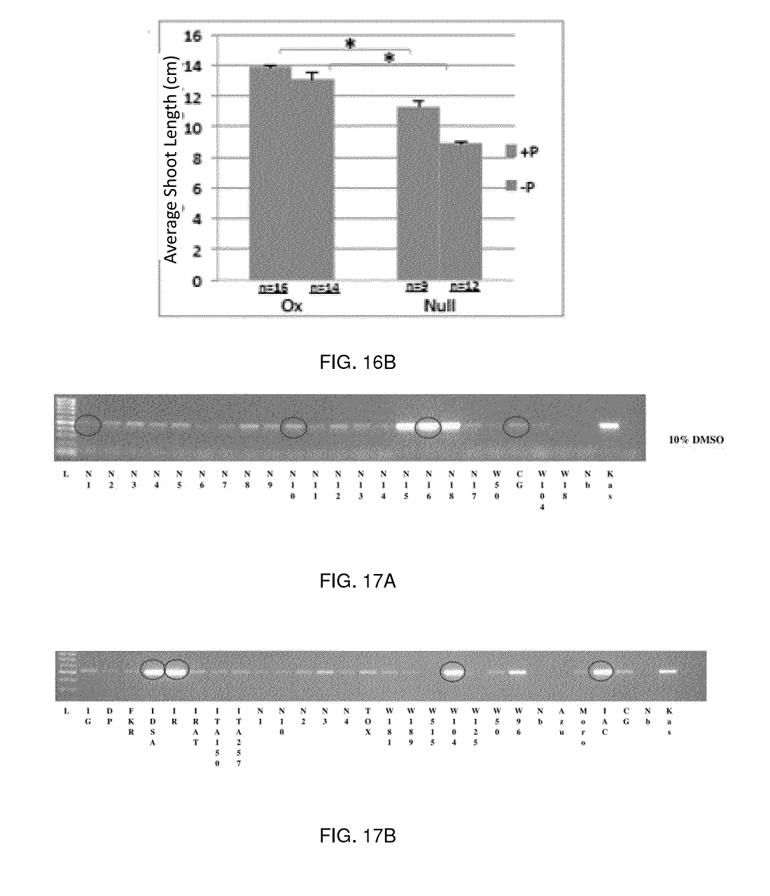

Dirigent overexpressors showed enhanced seedling vigor which is easily visible by large differences in plant height (FIG. 16). There was not much difference in shoot height at mature stage as compared with the large differences seen at seedling and early vegetative stage. Transgenic plants show at least 10% more fertility than their corresponding Nulls in three independent lines. The filled grain of the transgenic plant in lines 4c and 12a was more than double that of their corresponding Nulls.

Semi-quantitative RT-PCR analysis was done on OsPupk20-2 (dirigent) overexpression lines to check expression levels. Transgenic lines with moderate and low levels of expression show greater phenotypic differences when compared to corresponding nulls.

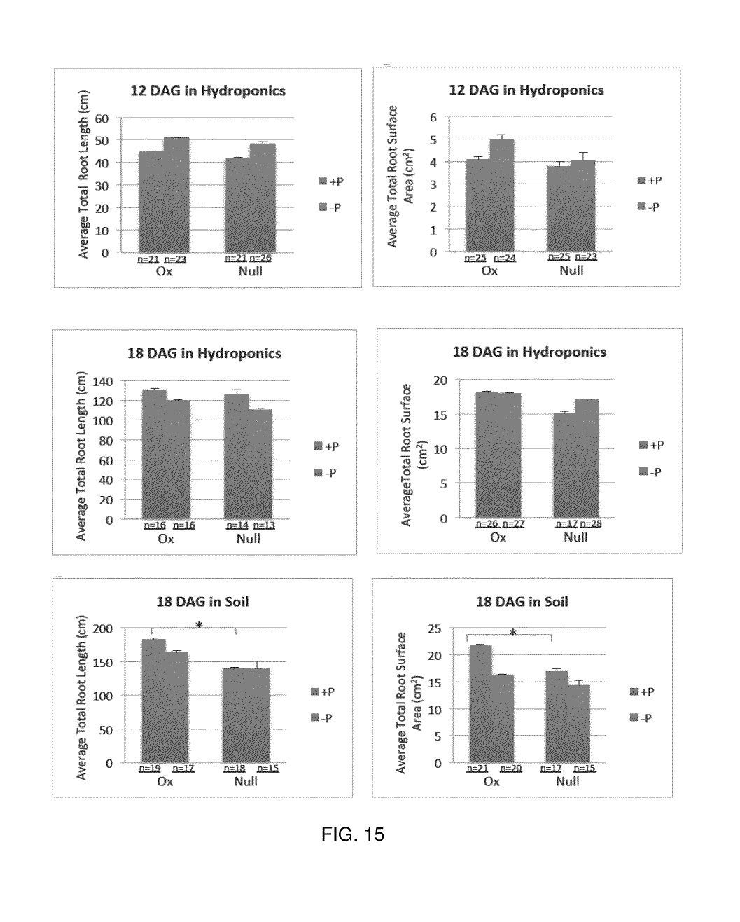

Root scan analysis was done on OsPupk20-2 (dirigent) overexpression lines and corresponding Nulls grown in +P/−P soil and +P/−P hydroponics at 12 and 18 DAG (FIG. 15). In hydroponics, overexpressors showed higher total root surface area and higher shoot length in +P/−P at 12DAG. At 18DAG in hydroponics, overexpressors showed higher total root length as compared to nulls in +P/−P. In soil, overexpressors had higher total root length and total surface area in +P or −P (FIG. 15).

Additional measurements were taken and it was found that transgenic shoot lengths were higher at 10,12,16 DAG in +P soil.

### Example 3

A novel allele of the P-starvation tolereance gene OsPSTOL1 from African rice (Oryza glaberrima Steud) and its distribution in the genus Oryza

Plant Material

Seeds of rice varieties and wild Oryza species were obtained from IRRI, AfricaRice and JIRCAS germplasm bank. Seeds were surface sterilized with sodium hypochlorite, rinsed and incubated for 2-3 days at 30° C. The germinated seeds were then transferred to a mesh floating on Yoshida nutrient solution [containing at full strength: N 2.86 mM (as NO3NH4), P 0.05 mM, K 1 mM, Ca 1 mM, Mg 1 mM, Mn 9 μM, Mo 0.5 μM, B 18.5 μM, Cu 0.16 μM, Fe 36 μM, Zn 0.15 μM]. The nutrient solution (half-strength) was replaced weekly, until leaf samples were taken at the third week.

DNA Extraction

Small pieces of leaves tissue were flash-frozen in liquid nitrogen and kept at −80° C. until analyzed. The frozen tissue was disrupted using a Qiagen mixer mill (Retsch MM 300, Germany), and tungsten carbide beads for 1 min at 25 pulses s−1. Afterwards, DNA was extracted using the DNeasy Plant Mini Kit (Qiagen), following the manufacturers protocol. The leaf tissue was homogenized in the presence of kit buffers, the homogenate was passed through spin columns and treated with RNase-A (Qiagen). DNA was eluted using TE buffer (10 mM Tris—HCl and 0.5 mM EDTA, pH 9.0), quantified by OD using a Nanodrop spectrophotometer (Thermo Scientific, USA), and DNA integrity was confirmed by electrophoresis in 2.0% agarose gel.

Genotyping

PCR reactions were performed using genomic DNA (25 ng), pairs of gene-specific primers, and Taq polymerase (Takara, Japan). PCR thermal conditions were as follows: first denaturing step at 94° C. for 2 min, followed by 30 cycles of 94° C. for 30 s, 55-60° C. for 30 s, and 72° C. for 90 s, and concluded by an extension step at 72° C. for 10 min. A part of the primer set used in this study was reported previously by Chin et al. (2010), including co-dominant markers K05, K20, K29-1, K29-3, and dominant markers K41, K42, K43, K45, K46-1, K48, K52 and K59 (located in the Kasalath-specific INDEL region). Polymorphism among the genotypes was detected by electrophoresis of the PCR products. Alleles were coded based on similarity to Kasalath (K), Nipponbare (N), CG-14 (CG), missing (M), or unknown genotype allele (U).

Cloning, Sequencing, and Alignments

The PCR products were gel purified using a spin-column (Promega, Madison), ligated into the pGEM-T Easy Vector (Promega, Madison), and the ligated product was used to transform Escherichia coli JM109 competent cells (Takara, Japan) following the manufacturer's instructions. The plasmid DNA of positive clones was extracted using the PureYield™ Plasmid Miniprep System (Promega, Madison), and their sequence determined using the pUC/M13 forward or gene-specific primers. The amplicon identity was confirmed by nucleotide similarity using the Basic Local Alignment Search Tool (BLAST) software.

The O. glaberrima (IRGC accession #96717, variety name CG14) genome sequence was obtained from the Arizona Genomics Institute of the University of Arizona (ftp://glabgenome@ftp.genome.arizona.edu/), and local BLAST was performed using the program BioEdit (http://www.mbio.ncsu.edu/bioedit/bioedit.html, version 7.1.11) and/or gramene (http://www.gramene.org/). Sequence alignment was performed using the MAFFT 7 software (http://mafft.cbrcjp/alignment/server/index.html), and Jalview (http://wwwj alvi ew.org/).

Transcript Abundance Analysis

In order to analyze the transcript abundance of the Kasalath and CG14 alleles, genotypes harboring each allele were selected and grown in soil or hydroponic culture. Pots were filled with either P-deficient or P-fertilized soil as previously described by Pariasca-Tanaka et al. (2009). Seeds were sown directly in the soil to facilitate normal root development, and watering was carried out regularly to simulate upland condition. In hydroponic experiments, pre-germinated seeds were placed on a floating mesh for 1 week, and afterwards seedlings were transferred to 12-L containers with Yoshida nutrient solution (as described above) at two P-treatments: 2 μM P (low P) or 50 μM P (control, sufficient P). Tissue samples were taken 40 days after sowing (DAS); shoots (leaf blades) and roots were rapidly frozen in liquid nitrogen and stored at −70 ° C. until analyzed.

RNA Extraction and RT-PCR

Total RNA was extracted using the RNeasy Mini Kit (Qiagen, USA) according to the manufacturer's instruction as previously described by Pariasca-Tanaka et al. (2009). Total RNA (around 400 ng) was reverse transcribed (RT) using OligodT and Random 6-monomer primers, and a PrimeScript RT Enzyme Mix I (Takara, Japan) at 37° C. for 15 min, followed by a inactivation of the enzyme at 85° C. for 5 s, and storage at 4° C. Subsequently, allele-specific primers, Taq polymerase (Takara, Japan), and the first-strand cDNA were used for PCR. PCR thermal condition was as follows: first denaturing step at 94° C. for 2 min, followed by 30 cycles of 94 ° C. for 30 s, 55-60° C. for 30 s, and 72° C. for 90 s, and concluded by an extension step at 72° C. for 10 min.

Results

Example 1 showed that the protein kinase PSTOL1 is the major gene responsible for enhanced P uptake conferred by the Pup1 locus. To survey the presence of PSTOL1 in NERICAs and other important African varieties, PSTOL1-specific PCR-based molecular markers that were designed based on the sequence of the Pup1 donor variety Kasalath were initially used. Although the amplified DNA fragments had the expected size (523 bp) in all samples analyzed, strong bands were obtained in only a few samples, the majority showing weak bands (FIG. 17). This experiment was repeated several times with inconsistent results. Therefore, several of these weak bands were sequenced and the sequences aligned with the Kasalath PSTOL1 (OsPupK46-2).

The Kasalath amplicon, which was included as a positive control, showed 100% sequence identity with the Kasalath sequence deposited in the gene bank with Accession number AB458444. Of the 11 additional genotypes sequenced, four aligned perfectly to the Kasalath sequence. This included WAB56-104 (short: W104), the parent of NERICA1 to NERICA11. In contrast, the sequence of CG14, the O. glaberrima parent of NERICAs, had 14 nucleotide substitutions relative to the Kasalath sequence. Several of the tested NERICAs (N1, N2, N4, N6, N10) shared most substitutions with CG14, suggesting the presence of an O. glaberrima-specific PSTOL1 allele that NERICA varieties had inherited from CG14. However, a few nucleotide substitutions were only detected in CG14 and in one or two NERICAs. These may represent recent mutations in the second PSTOL1 allele or slight differences between parental accessions used in this study and in making the crosses resulting in NERICA varieties.

Comparison between PSTOL1 and the O. glaberrima gene model.

The presence of an O. glaberrima-specific PSTOL1 allele was verified in the O. glaberrima (CG14) genome sequence obtained from the Arizona Genomics Institute (AGI) website (Oryza glaberrima genome assembly version 1). The CG14 sequence had been assembled against the Nipponbare reference genome, which lacks PSTOL1 and the adjacent sequences of a large (˜90 kb) Pup1—specific insertion—eletion. PSTOL1 was detected in an unanchored scaffold (Oglab12_unplaced142) derived from the chromosome 12 pool 6 (96% sequence identity), rather than in assembled CG14 genome sequence.

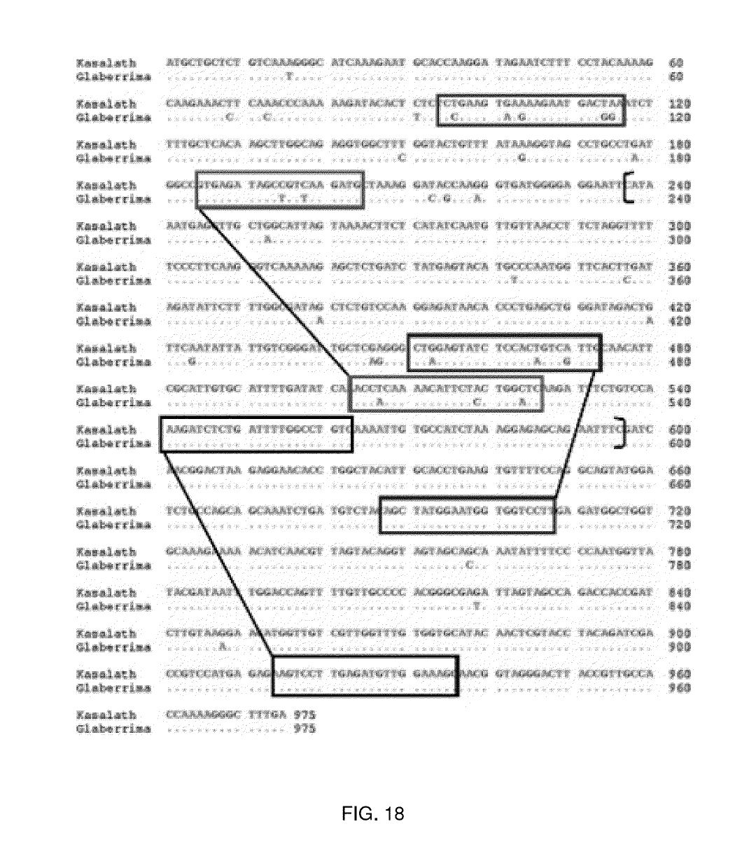

A sequence comparison between PSTOL1 with the corresponding CG14 gene (position: 116753-115779 by on ‘Oglab12_unplaced142’) revealed 35 nucleotide substitutions within the 975-bp sequence (FIG. 18), confirming the presence of an O. glaberrima PSTOL1 allele.

Development of duplex PCR.

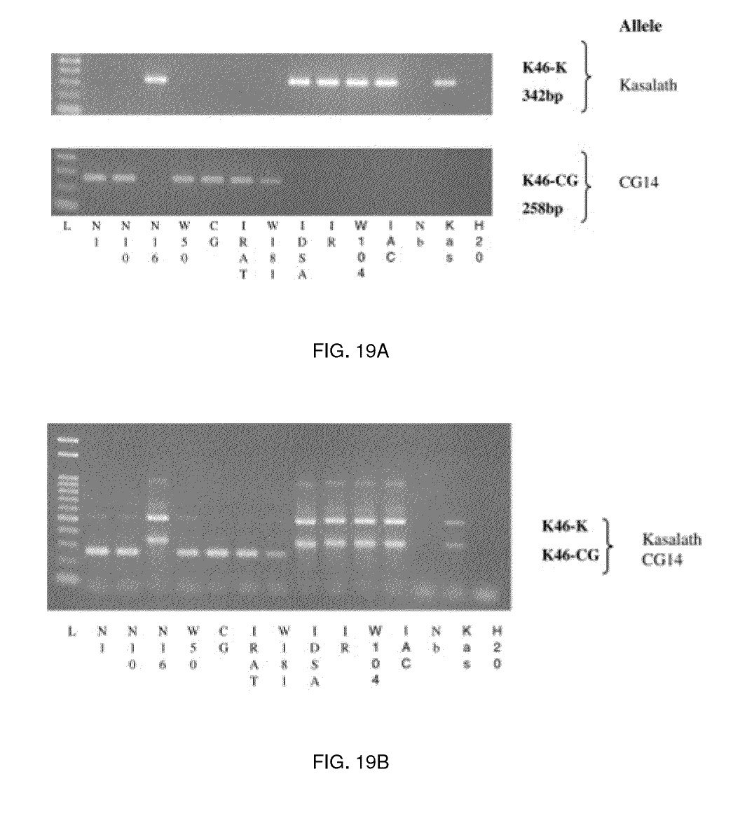

The initial screening of potential recipient cultivars was hampered by the erratic presence of weak bands during PCR amplification with PSTOL1 primers. This can be explained by the presence of two nucleotide substitutions in the CG14 allele located within the binding site for the forward primer of the marker ‘K46-1’ (FIG. 18). New primers were designed specifically targeting nucleotide polymorphisms between the two PSTOL1 alleles (FIG. 18; Table 3). Best results were obtained with primer pairs K46-K, which specifically amplifies the Kasalath PSTOL1 allele, and with primer pair K46-CG, specific for the CG14 allele (FIG. 19A). Additional allele-specific primers were developed (Table 3), which may be useful for the genotyping of specific genetic backgrounds, or if amplicons of different sizes are required.

TABLE 3
Design of Kasalath and CG14 allele specific
markers for genotyping diverse genotypes

| | | Sequence | SEQ | Sequence | SEQ | |
|---|---|---|---|---|---|---|
| Marker | | (forward | ID | (reverse | ID | Size |
| Name | Allele | primer) | NO: | primer) | NO: | (bp) |
| K46-1^{a} | Kas | K46-1 | 57 | K46-1 | 58 | 523 |
| | | TGAGATAGCCG | | AAGGACCACCAT | | |
| | | TCAAGATGCT | | TCCATAGC | | |
| K46-K1 | Kas | K46-1 | 57 | K46-Ksp3rv | 59 | 342 |
| | | TGAGATAGCCG | | TGAGCCAGTAGA | | |
| | | TCAAGATGCT | | ATGTTTTGAGG | | |
| K46-CG1 | CG | K46-CGsp2fw | 60 | K46-1 | 58 | 258 |
| | | CTAGAGTATCT | | AAGGACCACCAT | | |
| | | CCACAGTCGTT | | TCCATAGC | | |
| K46-K2 | Kas | K46-Ksp4fw | 61 | K46-Ksp3rv | 62 | 433 |
| | | CTGAAGTGAAA | | TGAGCCAGTAGA | | |
| | | AGAATGACTAA | | ATGTTTTGAGG | | |
| K46-CG2 | CG | K46-CGsp4fw | 63 | K46-CGsp3rv | 64 | 433 |
| | | CCGAAGTAAGA | | TGATCCAGGAGA | | |
| | | AGAATGACGGA | | ATGTTTTGTGG | | |
| K46-3 | Kas/CG | K46-3 | 65 | K46-3 | 66 | 400 |
| | | TCCAAAGATCT | | GCTTTCCAACAT | | |
| | | CTGATTTTGGC | | CTCAAGGACT | | |

^{a}Chin et al. 2011

One additional feature of the developed allele-specific markers is that they can be combined in a duplex PCR. The size difference of the amplicons is sufficiently large (342 vs. 258 bp) to differentiate the distinct PSTOL1 alleles in a single PCR reaction and subsequent gel electrophoresis (FIG. 19B). A single band (258 bp) is indicative of the CG14 allele, whereas the Kasalath PSTOL1 allele is indicated by the expected 342-bp amplicon and a second amplicon (˜500 bp) derived from the K46-1 primer pair that is reconstituted in the duplex assay. Other primer combinations did not produce clear diagnostic band pattern.

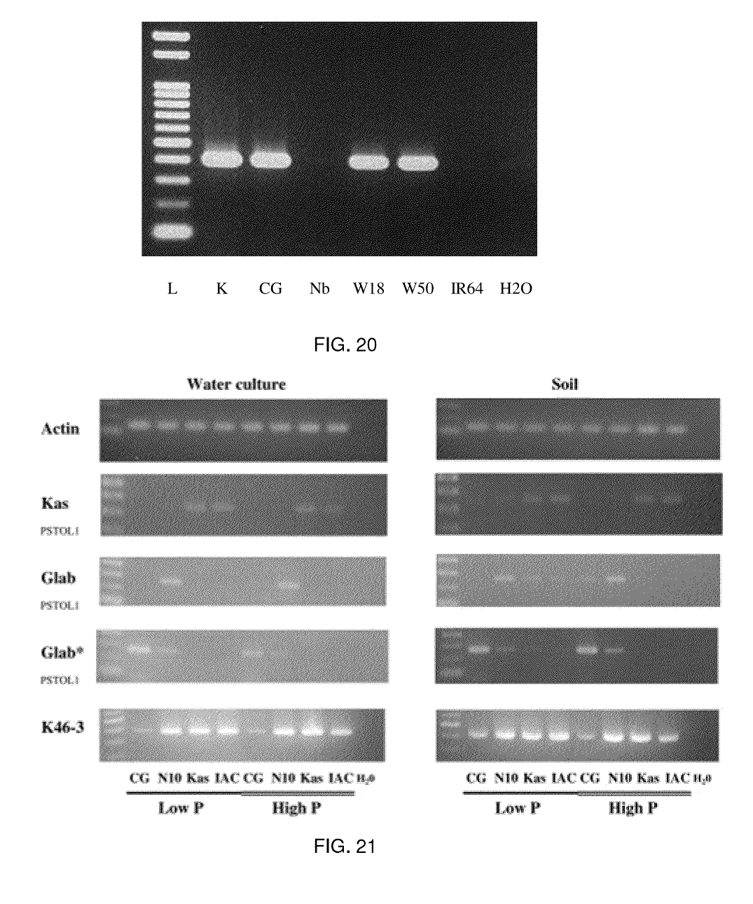

In addition to the allele-specific markers, marker K46-3 was designed, which targets a conserved region (FIG. 18; Table 3) and amplifies both PSTOL1 alleles equally (FIG. 20).

Assessing the presence of PSTOL1 alleles across cultivated and wild rice.

The duplex marker system was further employed to determine the PSTOL1 allele across a wide selection of cultivated and wild Oryza accessions. Among the 76 O. sativa accessions tested, PSTOL1 was absent from 26 accessions, whereas 23 accessions possessed the Kasalath and 27 possessed the CG14 allele (Table 4). Within the O. sativa sub-groups, indica type most commonly lacked the PSTOL1 gene, whereas aus-type accessions mainly had the Kasalath allele. Tropical japonica accessions were common in all three groups.

TABLE 4
Distribution of OsPSTOL1 allele in the genus Oryza

| | | | Allele | | | |
|---|---|---|---|---|---|---|
| Species | Genome | n | K | CG | Novel | Absent |
| O. sativa | AA | 76 | 23 | 27 | — | 26 |
| O. glaberrima | AA | 44 | 2 | 39 | 3 | — |
| O. nivara | AA | 6 | 2 | 1 | 2 | 1 |
| O. rufipogon | AA | 10 | 3 | 1 | 3 | 3 |
| O. longistaminata | AA | 6 | 5 | — | 1 | — |
| O. barthii | AA | 10 | 1 | 9 | — | — |
| O. glumaepatula | AA | 3 | 1 | — | — | 2 |
| O. meridionalis | AA | 3 | — | 2 | — | 1 |
| O. punctata | BB | 5 | — | — | — | 5 |
| O. officinalis | CC | 11 | — | — | — | 11 |
| O. australiensis | EE | 4 | — | — | — | 4 |
| O. brachyantha | FF | 1 | — | — | — | 1 |
| O. granulata | GG | 4 | — | — | — | 4 |
| O. minuta | BBCC | 5 | — | — | — | 5 |
| O. alta | CCDD | 4 | — | — | — | 4 |
| O. grandiglumis | CCDD | 2 | — | — | — | 2 |
| O. ridleyi | HHJJ | 1 | — | — | — | 1 |

PCR was performed using allele-specific markers for genotyping

In contrast, O. glaberrima accessions (44) predominantly had the CG14 allele, with only two accessions showing the Kasalath allele and three having no amplification (Table 4). The O. glaberrima CG14 allele was also predominantly present in interspecific NERICA upland accessions, but mostly absent in lowland NERICA (NERICA L) accessions with the exception of the group NERICA L23, L24, and L25, which all contained the CG14 allele. NERICA15, 16 and 18 varieties showed the Kasalath allele despite both parents (CG14 and WAB181-18) lacking the CG14 allele. Similarly, NERICA L15, L28, L29 and others showed the Kasalath allele despite this allele being absent in both parents (TOG5681 has the CG14 allele while IR64 completely lacks the gene). These genotypes may be explained by the presence of non-parental introgressions, which have been reported in NERICAs. Inconsistencies were also detected in WAB56-104, the sativa parent of NERICA1-11: the accession from AfricaRice showed the Kasalath allele, but a different accession in use at JIRCAS was genotyped as having the CG14 allele.

Among the important African rice varieties, the majority had the CG14 allele and only one cultivar (WAB515-B-16-A2-2) lacked PSTOL1.

Additionally, a set of 76 wild rice accessions was genotyped using the PSTOL1 allele-specific markers. Within O. nivara and O. rufipogon, the ancestors of O. sativa, no consistent genotype was detected since the PSTOL1 gene was either absent or present as Kasalath or CG14 allele (Table 4). In addition, a putative novel allele was detected which was amplified with both the Kasalath and CG14-specific markers. This allele was also detected in O. longistaminata (Table 4), but not in O. barthii, the O. glaberrima ancestor. Nine out of ten O. barthii accessions showed amplification with the O. glaberrima CG14 allele-specific marker. The presence of PSTOL1 was restricted to Oryza species belonging to the AA genome, since no allele could be detected in accessions with BB˜HHJJ genomes (Table 4).

Transcript Abundance

Genotypes harboring the Kasalath allele (Kasalath, and IAC165) or the CG14 allele (CG14 and NERICA10) were grown in P-deficient and P-replete conditions to determine their PSTOL1 transcript abundance. An RT-PCR analysis using total RNA extracted from roots and allele-specific and unspecific primers confirmed the existence of the CG14 allele at the transcript level (FIG. 21). Expression of both alleles appeared to be constitutive and not noticeably enhanced by P deficiency. The abundance of the CG14 transcript in NERICA10 was comparable to that of the Kasalath allele (Kasalath and IAC165) in both water culture and soil. PSTOL1 transcript abundance was very low in CG14, irrespective of the growth conditions (FIG. 21). The results obtained with the allele-specific markers were also confirmed with the marker K46-3, which is located in the conserved region (FIG. 21). Furthermore, in-silico analysis of the protein sequence indicated that the kinase catalytic domain was conserved in the CG14 allele. Overall amino acid sequence similarity between both alleles was 94.1%.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A method of improving root growth and nutrient uptake in a cereal grass comprising:
a) crossing a crossing plant of one variety of cereal grass having chromosomal DNA that includes at least one polynucleotide sequence at least 70% identical to that of SEQ ID NO: 3 (OsPupK20-2), at least 70% identical to that of SEQ ID NO: 5 (OsPSTOL1), or both, with a recipient plant of a distinct variety of cereal grass having chromosomal DNA that does not include a polynucleotide sequence at least 70% identical to that of SEQ ID NO: 3 (OsPupK20-2), at least 70% identical to that of SEQ ID NO: 5 (OsPSTOL1), or both; and
b) selecting one or more progeny plants having chromosomal DNA that includes at least one polynucleotide sequence at least 70% identical to that of SEQ ID NO: 3 (OsPupK20-2), at least 70% identical to that of SEQ ID NO: 5 (OsPSTOL1), or both.

2. The method of claim 1, further comprising the steps:
a) backcrossing the one or more selected progeny plants to produce backcross progeny plants; and
b) selecting one or more backcross progeny plants having chromosomal DNA that includes the at least one polynucleotide sequence at least 70% identical to that of SEQ ID NO: 3 (OsPupK20-2), at least 70% identical to SEQ ID NO: 5 (OsPSTOL1), or both.

3. The method of claim 2, wherein steps a) and b) are repeated one or more times to produce third or higher backcross progeny plants having chromosomal DNA that includes the at least one polynucleotide sequence at least 70% identical to that of SEQ ID NO: 3 (OsPupK20-2), at least 70% identical to that of SEQ ID NO: 5 (OsPSTOL1), or both, and the physiological and morphological characteristics of the recipient plant.

4. The method of claim 1, wherein the selected one or more progeny plants has increased booth growth relative to a control plant.

5. The method of claim 1, wherein the selected one or more progeny plants has increased booth growth relative to a control plant in both high- and low-phosphorus conditions.

6. The method of claim 1, wherein the selected one or more progeny plants has improved tolerance to phosphorus-deficiency relative to a control plant.

7. The method of claim 1, wherein the selected one or more progeny plants has increased uptake of one or more nutrients selected from the group consisting of: nitrogen; potassium; and phosphorus, relative to a control plant.

8. The method of claim 1, wherein the cereal grass is selected from the group consisting of: rice; corn; wheat; barley; sorghum; millet; oats; and rye.

9. The method of claim 1, wherein the cereal grass is rice.

10. The method of claim 1, wherein the cereal grass is corn.

11. The method of claim 1, wherein the crossing plant is a rice plant selected from the group consisting of: Kasalath; AUS 196; IRAT 77; Azucena; Pratao Precoce; Apo; Vary Lava 701; AUS 257; Dular;
IAC 25; IAC 47; UPL R17; UPL RI 5; Vandana; and Way Rarem.

12. The method of claim 1, wherein the crossing plant is a rice plant of variety Kasalath.

13. The method of claim 1, wherein the recipient plant is a rice plant selected from the group consisting of: IR 64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

14. The method of claim 1, wherein detection of SEQ ID NO: 5 (OsPSTOL1), or lack thereof, is performed using one or more markers selected from the group consisting of: K46-1; K46-K1; K46-CG1; K46-K2; K46-CG2; and K46-3.

15. The method of claim 1, wherein detection of SEQ ID NO: 5 (OsPSTOL1), or lack thereof is performed using marker K46-K1.

16. The method of claim 1, wherein detection of SEQ ID NO: 3 (OsPupK20-2), or lack thereof is performed using forward primer SEQ ID NO: 68 and reverse primer SEQ ID NO: 69.

17. A method for selecting a cereal grass plant having improved root growth and nutrient uptake relative to a control cereal grass plant, comprising:
a) inducing expression or increasing expression in a cereal grass plant at least one polynucleotide encoding at least one polypeptide having at least 70% sequence identity to an amino acid of SEQ ID NO: 8 (OsPupK20-2), at least 70% sequence identity to an amino acid of SEQ ID NO:10, or both; and
b) selecting a cereal grass plant having improved root growth and nutrient uptake relative to a control cereal grass plant, wherein the induced or increased expression of the at least one polynucleotide is obtained by transforming and expressing in the cereal grass plant the at least one polynucleotide.

18. The method of claim 17, wherein the selected cereal grass plant, in addition to improved root growth and nutrient uptake, has improved tolerance to phosphorus-deficiency.

19. The method of claim 17, wherein the induced or increased expression of the at least one polynucleotide is a result of introducing and expressing the at least one polynucleotide in the cereal grass plant under control of at least one promoter functional in plants.

20. The method of claim 19, wherein the at least one promoter and the at least one polypeptide are operably linked

21. The method of claim 17, wherein the cereal grass plant is selected from the group consisting of: rice; corn; wheat; barley; sorghum; millet; oats; and rye.

22. The method of claim 17, wherein the at least one polynucleotide encodes a polypeptide sequence having an identity selected from the group consisting of: at least 70% to SEQ ID NO: 8 (OsPupK20-2); at least 70% to SEQ ID NO: 10 (OsPSTOL1); at least 75% to SEQ ID NO: 8 (OsPupK20-2); at least 75% to SEQ ID NO: 10 (OsPSTOL1); at least 80% to SEQ ID NO: 8 (OsPupK20-2); at least 80% to SEQ ID NO: 10 (OsPSTOL1); at least 85% to SEQ ID NO: 8 (OsPupK20-2); at least 85% to SEQ ID NO: 10 (OsPSTOL1); at least 90% to SEQ ID NO: 8 (OsPupK20-2); at least 90% to SEQ ID NO: 10 (OsPSTOL1); at least 95% to SEQ ID NO: 8 (OsPupK20-2); at least 95% to SEQ ID NO: 10 (OsPSTOL1); at least 96% to SEQ ID NO: 8 (OsPupK20-2); at least 96% to SEQ ID NO: 10 (OsPSTOL1); at least 97% to SEQ ID NO: 8 (OsPupK20-2); at least 97% to SEQ ID NO: 10 (OsPSTOL1); at least 98% to SEQ ID NO: 8 (OsPupK20-2); at least 98% to SEQ ID NO: 10 (OsPSTOL1); at least 99% to SEQ ID NO: 8 (OsPupK20-2); at least 99% to SEQ ID NO: 10 (OsPSTOL1); at least 100% to SEQ ID NO: 8 (OsPupK20-2); and at least 100% to SEQ ID NO: 10 (OsPSTOL1).

23. The method of claim 17, wherein the at least one polynucleotide has a sequence identity selected from the group consisting of: at least 70% to SEQ ID NO: 3 (OsPupK20-2); at least 70% to SEQ ID NO: 5 (OsPSTOL1); at least 75% to SEQ ID NO: 3 (OsPupK20-2); at least 75% to SEQ ID NO: 5 (OsPSTOL1); at least 80% to SEQ ID NO: 3 (OsPupK20-2); at least 80% to SEQ ID NO: 5 (OsPSTOL1); at least 85% to SEQ ID NO: 3 (OsPupK20-2); at least 85% to SEQ ID NO: 5 (OsPSTOL1); at least 90% to SEQ ID NO: 3 (OsPupK20-2); at least 90% to SEQ ID NO: 5 (OsPSTOL1); at least 95% to SEQ ID NO: 3 (OsPupK20-2); at least 95% to SEQ ID NO: 5 (OsPSTOL1); at least 96% to SEQ ID NO: 3 (OsPupK20-2); at least 96% to SEQ ID NO: 5 (OsPSTOL1); at least 97% to SEQ ID NO: 3 (OsPupK20-2); at least 97% to SEQ ID NO: 5 (OsPSTOL1); at least 98% to SEQ ID NO: 3 (OsPupK20-2); at least 98% to SEQ ID NO: 5 (OsPSTOL1); at least 99% to SEQ ID NO: 3 (OsPupK20-2); at least 99% to SEQ ID NO: 5 (OsPSTOL1); at least 100% to SEQ ID NO: 3 (OsPupK20-2); and at least 100% to SEQ ID NO: 5 (OsPSTOL1).

24. A method for making a cereal grass plant having improved root growth and nutrient uptake relative to a control cereal grass plant comprising:
a) transforming a cereal grass plant cell, cereal grass plant, or part thereof with a construct comprising:
(1) a polynucleotide encoding a polypeptide having at least 70% sequence identity to an amino acid sequence selected from the group consisting of: SEQ ID NO: 8 (OsPupK20-2); and SEQ ID NO:10 (OsPSTOL1);
(2) a promoter operably linked to the polynucleotide; and
(3) a transcription termination sequence; and
b) expressing the construct in a cereal grass plant cell, cereal grass plant, or part thereof.

25. The method of claim 24, further comprising a step of selecting for a cereal grass plant having improved root growth relative to a control cereal grass plant.

26. The method of claim 24, further comprising a step of selecting for a cereal grass plant having increased uptake of one or more nutrients selected from the group consisting of: nitrogen; potassium;
and phosphorus, relative to a control plant.

27. The method of claim 24, further comprising a step of selecting for a cereal grass plant having improved tolerance of phosphorus-deficiency relative to a control cereal grass plant.

28. The method of claim 27 wherein the cereal grass plant displays a phenotype comprising one or more characteristics selected from the group consisting of: greater tolerance to soil phosphorus deficiency relative to a control grass plant; greater total root length relative to a control grass plant; greater root surface area relative to a control grass plant; greater total grain weight per plant relative to a control grass plant; early crown root development relative to a control grass plant; increased nutrient uptake relative to a control grass plant; increased nitrogen uptake relative to a control grass plant; increased potassium uptake relative to a control grass plant; increased phosphorus uptake relative to a control grass plant; increased grain yield relative to a control grass plant; and reduced spikelet sterility relative to a control grass plant.

29. The method of claim 24, wherein the cereal grass plant cell, cereal grass plant, or part thereof is selected from the group consisting of: rice; corn; wheat; barley; sorghum; millet; oats; and rye.

30. The method of claim 24, wherein the construct comprises one or more polynucleotides encoding a polypeptide having at least 70% sequence identity to SEQ ID NO: 8 (OsPupK20-2), at least 70% sequence identity to SEQ ID NO:10 (OsPSTOL1), or both.

31. The method of claim 24, wherein the polynucleotide encodes a polypeptide sequence having an identity selected from the group consisting of: at least 70% to SEQ ID NO: 8 (OsPupK20-2); at least 70% to SEQ ID NO: 10 (OsPSTOL1); at least 75% to SEQ ID NO: 8 (OsPupK20-2); at least 75% to SEQ ID NO: 10 (OsPSTOL1); at least 80% to SEQ ID NO: 8 (OsPupK20-2); at least 80% to SEQ ID NO: 10 (OsPSTOL1); at least 85% to SEQ ID NO: 8 (OsPupK20-2); at least 85% to SEQ ID NO: 10 (OsPSTOL1); at least 90% to SEQ ID NO: 8 (OsPupK20-2); at least 90% to SEQ ID NO: 10 (OsPSTOL1); at least 95% to SEQ ID NO: 8 (OsPupK20-2); at least 95% to SEQ ID NO: 10 (OsPSTOL1); at least 96% to SEQ ID NO: 8 (OsPupK20-2); at least 96% to SEQ ID NO: 10 (OsPSTOL1); at least 97% to SEQ ID NO: 8 (OsPupK20-2); at least 97% to SEQ ID NO: 10 (OsPSTOL1); at least 98% to SEQ ID NO: 8 (OsPupK20-2); at least 98% to SEQ ID NO: 10 (OsPSTOL1); at least 99% to SEQ ID NO: 8 (OsPupK20-2); at least 99% to SEQ ID NO: 10 (OsPSTOL1); at least 100% to SEQ ID NO: 8 (OsPupK20-2); and at least 100% to SEQ ID NO: 10 (OsPSTOL1).

32. The method of claim 24, wherein the construct comprises one or more polynucleotides having at least 70% sequence identity to SEQ ID NO: 3 (OsPupK20-2), at least 70% sequence identity to SEQ ID NO:10 (OsPSTOL1), or both.

33. The method of claim 24, wherein the polynucleotide has a sequence identity selected from the group consisting of: at least 70% to SEQ ID NO: 3 (OsPupK20-2); at least 70% to SEQ ID NO: 5 (OsPSTOL1); at least 75% to SEQ ID NO: 3 (OsPupK20-2); at least 75% to SEQ ID NO: 5 (OsPSTOL1); at least 80% to SEQ ID NO: 3 (OsPupK20-2); at least 80% to SEQ ID NO: 5 (OsPSTOL1); at least 85% to SEQ ID NO: 3 (OsPupK20-2); at least 85% to SEQ ID NO: 5 (OsPSTOL1); at least 90% to SEQ ID NO: 3 (OsPupK20-2); at least 90% to SEQ ID NO: 5 (OsPSTOL1); at least 95% to SEQ ID NO: 3 (OsPupK20-2); at least 95% to SEQ ID NO: 5 (OsPSTOL1); at least 96% to SEQ ID NO: 3 (OsPupK20-2); at least 96% to SEQ ID NO: 5 (OsPSTOL1); at least 97% to SEQ ID NO: 3 (OsPupK20-2); at least 97% to SEQ ID NO: 5 (OsPSTOL1); at least 98% to SEQ ID NO: 3 (OsPupK20-2); at least 98% to SEQ ID NO: 5 (OsPSTOL1); at least 99% to SEQ ID NO: 3 (OsPupK20-2); at least 99% to SEQ ID NO: 5 (OsPSTOL1); at least 100% to SEQ ID NO: 3 (OsPupK20-2); and at least 100% to SEQ ID NO: 5 (OsPSTOL1).

34. A method for the production of a transgenic cereal grass plant having improved root growth and nutrient uptake relative to a control cereal grass plant comprising:
a) transforming and expressing in a cereal grass plant cell at least one polynucleotide encoding at least one polypeptide having at least 70% sequence identity to an amino acid sequence of SEQ ID NO: 8 (OsPupK20-2), at least 70% sequence identity to an amino acid sequence of SEQ ID NO:10 (OsPSTOL1), or both; and
b) cultivating the cereal grass plant cell under conditions promoting plant growth and development, and obtaining transformed plants expressing OsPupK20-2, OsPSTOL1, or both.

35. The method of claim 34, further comprising a step of selecting for a cereal grass plant having improved root growth relative to a control cereal grass plant.

36. The method of claim 34, further comprising a step of selecting for a cereal grass plant having increased uptake of one or more nutrients selected from the group consisting of: nitrogen; potassium;
and phosphorus, relative to a control plant.

37. The method of claim 34, further comprising a step of selecting for a cereal grass plant having improved tolerance of phosphorus-deficiency relative to a control cereal grass plant.

38. The method of claim 34 wherein the cereal grass plant displays a phenotype comprising one or more characteristics selected from the group consisting of: greater tolerance to soil phosphorus deficiency relative to a control grass plant; greater total root length relative to a control grass plant; greater root surface area relative to a control grass plant; greater total grain weight per plant relative to a control grass plant; early crown root development relative to a control grass plant; increased nutrient uptake relative to a control grass plant; increased nitrogen uptake relative to a control grass plant; increased potassium uptake relative to a control grass plant; increased phosphorus uptake relative to a control grass plant; increased grain yield relative to a control grass plant; and reduced spikelet sterility relative to a control grass plant.

39. A transgenic plant cell comprising:
a) at least one promoter that is functional in plants; and
b) at least one polynucleotide encoding a polypeptide sequence at least 70% identical to an amino acid sequence of SEQ ID NO: 8 (OsPupK20-2), SEQ ID NO:10 (OsPSTOL1), or both,
wherein the promoter and polynucleotide are operably linked and incorporated into the plant cell chromosomal DNA.

40. The transgenic plant cell of claim 39, wherein the type of plant cell is selected from the group consisting of: rice plant cell; corn plant cell; wheat plant cell; barley plant cell; sorghum plant cell; millet plant cell; oats plant cell; and rye plant cell.

41. The transgenic plant cell of claim 39, wherein the polynucleotide encodes a polypeptide sequence having an identity selected from the group consisting of: at least 70% to SEQ ID NO: 8 (OsPupK20-2); at least 70% to SEQ ID NO: 10 (OsPSTOL1); at least 75% to SEQ ID NO: 8 (OsPupK20-2); at least 75% to SEQ ID NO: 10 (OsPSTOL1); at least 80% to SEQ ID NO: 8 (OsPupK20-2); at least 80% to SEQ ID NO: 10 (OsPSTOL1); at least 85% to SEQ ID NO: 8 (OsPupK20-2); at least 85% to SEQ ID NO: 10 (OsPSTOL1); at least 90% to SEQ ID NO: 8 (OsPupK20-2); at least 90% to SEQ ID NO: 10 (OsPSTOL1); at least 95% to SEQ ID NO: 8 (OsPupK20-2); at least 95% to SEQ ID NO: 10 (OsPSTOL1); at least 96% to SEQ ID NO: 8 (OsPupK20-2); at least 96% to SEQ ID NO: 10 (OsPSTOL1); at least 97% to SEQ ID NO: 8 (OsPupK20-2); at least 97% to SEQ ID NO: 10 (OsPSTOL1); at least 98% to SEQ ID NO: 8 (OsPupK20-2); at least 98% to SEQ ID NO: 10 (OsPSTOL1); at least 99% to SEQ ID NO: 8 (OsPupK20-2); at least 99% to SEQ ID NO: 10 (OsPSTOL1); at least 100% to SEQ ID NO: 8 (OsPupK20-2); and at least 100% to SEQ ID NO: 10 (OsPSTOL1).

42. The transgenic plant cell of claim 39, wherein the at least one polynucleotide has at least 70% sequence identity to SEQ ID NO: 3 (OsPupK20-2), at least 70% sequence identity to SEQ ID NO:10 (OsPSTOL1), or both

43. The transgenic plant cell of claim 42, wherein the polynucleotide has a sequence identity selected from the group consisting of: at least 70% to SEQ ID NO: 3 (OsPupK20-2); at least 70% to SEQ ID NO: 5 (OsPSTOL1); at least 75% to SEQ ID NO: 3 (OsPupK20-2); at least 75% to SEQ ID NO: 5 (OsPSTOL1); at least 80% to SEQ ID NO: 3 (OsPupK20-2); at least 80% to SEQ ID NO: 5 (OsPSTOL1); at least 85% to SEQ ID NO: 3 (OsPupK20-2); at least 85% to SEQ ID NO: 5 (OsPSTOL1); at least 90% to SEQ ID NO: 3 (OsPupK20-2); at least 90% to SEQ ID NO: 5 (OsPSTOL1); at least 95% to SEQ ID NO: 3 (OsPupK20-2); at least 95% to SEQ ID NO: 5 (OsPSTOL1); at least 96% to SEQ ID NO: 3 (OsPupK20-2); at least 96% to SEQ ID NO: 5 (OsPSTOL1); at least 97% to SEQ ID NO: 3 (OsPupK20-2); at least 97% to SEQ ID NO: 5 (OsPSTOL1); at least 98% to SEQ ID NO: 3 (OsPupK20-2); at least 98% to SEQ ID NO: 5 (OsPSTOL1); at least 99% to SEQ ID NO: 3 (OsPupK20-2); at least 99% to SEQ ID NO: 5 (OsPSTOL1); at least 100% to SEQ ID NO: 3 (OsPupK20-2); and at least 100% to SEQ ID NO: 5 (OsPSTOL1).

44. The transgenic plant cell of claim 39, wherein the plant cell is homozygous for the at least one polynucleotide.

45. A transgenic plant comprising a plurality of transgenic plant cells, wherein the transgenic plant cells comprise:
a) at least one promoter that is functional in plants; and
b) at least one polynucleotide encoding a polypeptide sequence at least 70% identical to an amino acid sequence of SEQ ID NO: 8 (OsPupK20-2), SEQ ID NO:10 (OsPSTOL1), or both,
wherein the promoter and polynucleotide are operably linked and incorporated into the plant cell chromosomal DNA.

46. A transgenic plant comprising:
a) at least one promoter functional in plants; and
b) at least one polynucleotide encoding a polypeptide sequence at least 70% identical to an amino acid sequence of SEQ ID NO: 8 (OsPupK20-2), SEQ ID NO:10 (OsPSTOL1), or both,
wherein the promoter and polynucleotide are operably linked and incorporated into the plant cell chromosomal DNA.

47. The transgenic plant of claim 46, wherein the plant is selected from the group consisting of: rice; corn; wheat; barley; sorghum; millet; oats; and rye.

48. The transgenic plant of claim 46, wherein the polynucleotide encodes a polypeptide sequence having an identity selected from the group consisting of: at least 70% to SEQ ID NO: 8 (OsPupK20-2); at least 70% to SEQ ID NO: 10 (OsPSTOL1); at least 75% to SEQ ID NO: 8 (OsPupK20-2); at least 75% to SEQ ID NO: 10 (OsPSTOL1); at least 80% to SEQ ID NO: 8 (OsPupK20-2); at least 80% to SEQ ID NO: 10 (OsPSTOL1); at least 85% to SEQ ID NO: 8 (OsPupK20-2); at least 85% to SEQ ID NO: 10 (OsPSTOL1); at least 90% to SEQ ID NO: 8 (OsPupK20-2); at least 90% to SEQ ID NO: 10 (OsPSTOL1); at least 95% to SEQ ID NO: 8 (OsPupK20-2); at least 95% to SEQ ID NO: 10 (OsPSTOL1); at least 96% to SEQ ID NO: 8 (OsPupK20-2); at least 96% to SEQ ID NO: 10 (OsPSTOL1); at least 97% to SEQ ID NO: 8 (OsPupK20-2); at least 97% to SEQ ID NO: 10 (OsPSTOL1); at least 98% to SEQ ID NO: 8 (OsPupK20-2); at least 98% to SEQ ID NO: 10 (OsPSTOL1); at least 99% to SEQ ID NO: 8 (OsPupK20-2); at least 99% to SEQ ID NO: 10 (OsPSTOL1); at least 100% to SEQ ID NO: 8 (OsPupK20-2); and at least 100% to SEQ ID NO: 10 (OsPSTOL1).

49. The transgenic plant of claim 46, wherein the at least one polynucleotide has at least 70% sequence identity to SEQ ID NO: 3 (OsPupK20-2), at least 70% sequence identity to SEQ ID NO:10 (OsPSTOL1), or both.

50. The transgenic plant of claim 49, wherein the polynucleotide has a sequence identity selected from the group consisting of: at least 70% to SEQ ID NO: 3 (OsPupK20-2); at least 70% to SEQ ID NO: 5 (OsPSTOL1); at least 75% to SEQ ID NO: 3 (OsPupK20-2); at least 75% to SEQ ID NO: 5 (OsPSTOL1); at least 80% to SEQ ID NO: 3 (OsPupK20-2); at least 80% to SEQ ID NO: 5 (OsPSTOL1); at least 85% to SEQ ID NO: 3 (OsPupK20-2); at least 85% to SEQ ID NO: 5 (OsPSTOL1); at least 90% to SEQ ID NO: 3 (OsPupK20-2); at least 90% to SEQ ID NO: 5 (OsPSTOL1); at least 95% to SEQ ID NO: 3 (OsPupK20-2); at least 95% to SEQ ID NO: 5 (OsPSTOL1); at least 96% to SEQ ID NO: 3 (OsPupK20-2); at least 96% to SEQ ID NO: 5 (OsPSTOL1); at least 97% to SEQ ID NO: 3 (OsPupK20-2); at least 97% to SEQ ID NO: 5 (OsPSTOL1); at least 98% to SEQ ID NO: 3 (OsPupK20-2); at least 98% to SEQ ID NO: 5 (OsPSTOL1); at least 99% to SEQ ID NO: 3 (OsPupK20-2); at least 99% to SEQ ID NO: 5 (OsPSTOL1); at least 100% to SEQ ID NO: 3 (OsPupK20-2); and at least 100% to SEQ ID NO: 5 (OsPSTOL1).

51. The transgenic plant of claim 46, wherein the transgenic plant is homozygous for the at least one polynucleotide.

52. A seed of a plant of claim 46.

53. A plant part of a plant of claim 46.

54. The transgenic plant of claim 46, wherein said plant exhibits a phenotype selected from the group consisting of: greater tolerance to soil phosphorus deficiency relative to a corresponding non-ransgenic plant; greater total root length relative to a corresponding non-transgenic plant; greater root surface area relative to a corresponding non-transgenic plant; greater total grain weight per plant relative to a corresponding non-transgenic plant; early crown root development relative to a corresponding non-transgenic plant; increased nutrient uptake relative to a corresponding non-transgenic plant; increased nitrogen uptake relative to a corresponding non-transgenic plant; increased potassium uptake relative to a corresponding non-transgenic plant; increased phosphorus uptake relative to a corresponding non-transgenic plant; increased grain yield relative to a corresponding non-transgenic plant; and reduced spikelet sterility relative to a corresponding non-transgenic plant.

55. A method for selecting transgenic plants having improved root growth and nutrient uptake relative to a control plant, comprising:
a) screening a population of plants for increased root growth and nutrient uptake, wherein plants in the population comprise a transgenic plant cell having recombinant DNA incorporated into its chromosomal DNA, wherein the recombinant DNA comprises a promoter that is functional in a plant cell and that is functionally linked to an open reading frame of a polynucleotide encoding a polypeptide sequence at least 70% identical to SEQ ID NO: 8 (OsPupK20-2) and/or SEQ ID NO: 10 (OsPSTOL1), wherein individual plants in said population that comprise the transgenic plant cell exhibit phosphorous-deficiency tolerance at a level the same as or greater than a level of phosphorous-deficiency tolerance in control plants which do not comprise the transgenic plant cell; and
b) selecting from said population one or more plants that exhibit root growth and nutrient uptake at a level greater than the level of root growth and nutrient uptake in control plants which do not comprise the transgenic plant cell.

56. The method of claim 55, which further comprises selecting one or more plants that exibit tolerance of phosphorus-deficiency at a level greater than the level of tolerance of phosphorus-deficiency in control plants that do not comprise the transgenic plant cell.

57. The method of claim 55, which further comprises a step of collecting seeds from the one or more plants selected in step b).

58. The method of claim 55, wherein said method for selecting said transgenic seed further comprises:
a) verifying that said recombinant DNA is stably integrated in said selected plant; and
b) analyzing tissue of said selected plant to determine the expression of a polypeptide having a sequence at least 70% identical to SEQ ID NO: 8 (OsPupK20-2), SEQ ID NO: 10 (OsPSTOL1), or both.

59. The method of claim 55, wherein the plant is selected from the group consisting of: rice; corn; wheat; barley; sorghum; millet; oat; and rye.

60. The method of claim 55, wherein the plant is rice.

61. The method of claim 60, wherein the plant is a rice variety selected from the group consisting of: IR 64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, Nipponbare, IR20, and IR66.

62. The method of claim 55, wherein the plant is corn.
